(19) United States
(12) Patent Application Publication
(10) Pub. No.: US 2018/0132722 A1
(43) Pub. Date: May 17, 2018

(54) METHOD, APPARATUS AND SYSTEM FOR COMPLETE EXAMINATION OF TISSUE WITH HAND-HELD IMAGING DEVICES

(71) Applicants: Philip E. EGGERS, Amelia Island, FL (US); Scott P. HUNTLEY, Danville, CA (US); Eric A. EGGERS, Portland, OR (US); Bruce A. ROBINSON, Maple Valley, WA (US)

(72) Inventors: Philip E. EGGERS, Amelia Island, FL (US); Scott P. HUNTLEY, Danville, CA (US); Eric A. EGGERS, Portland, OR (US); Bruce A. ROBINSON, Maple Valley, WA (US)

(21) Appl. No.: 15/635,111

(22) Filed: Jun. 27, 2017

Related U.S. Application Data
(63) Continuation of application No. 13/854,800, filed on Apr. 1, 2013, now abandoned.
Application No. 13/854,800 is a continuation of application No. PCT/US2012/059176, filed on Oct. 8, 2012.
(60) Provisional application No. 61/647,180, filed on May 15, 2012.
(60) Provisional application No. 61/545,278, filed on Oct. 10, 2011.

(51) Int. Cl. A61B 5/00; G01S 15/89; G01S 7/52; A61B 8/00; A61B 8/08; A61B 8/14

(52) U.S. Cl. CPC A61B 5/0033; G01S 15/8936; G01S 7/5205; A61B 8/4444; A61B 8/4245; A61B 8/483; A61B 8/4405; A61B 8/0825; A61B 8/14; A61B 8/463; A61B 8/466; A61B 8/4254

(57) ABSTRACT

A scan completeness auditing system for screening a volume of tissue comprising a manual image scanning device having an imaging probe, a position tracking system configured to track and record the position of the imaging probe during use, and a controller in communication with the recording system and the manual image scanning device, the controller configured to electronically receive and record the scanned images from the manual image scanning device, and to measure an image-to-image spacing and a scan-to-scan spacing between the scanned images within scan sequence and between scan sequences respectively. The scan completeness auditing system is further adapted to provide an alert to the operator if the image-to-image or scan-to-scan spacing exceeds an acceptable value.

### CROSS REFERENCE TO RELATED APPLICATIONS

This application is a continuation of U.S. application Ser. No. 13/854,800, filed Apr. 1, 2013. Which is a continuation of International Application No. PCT/US2012/059176 filed on Oct. 8, 2012, which claims the benefit of U.S. Provisional Patent Application No. 61/545,278, filed Oct. 10, 2011 and U.S. Provisional Patent Application No. 61/647,180, filed May 15, 2012, each of which is herein incorporated by reference in its entirety.

### INCORPORATION BY REFERENCE

All publications and patent applications mentioned in this specification are herein incorporated by reference to the same extent as if each individual publication or patent application was specifically and individually indicated to be incorporated by reference.

Embodiments described relate generally to medical imaging and methods and devices for ensuring adequate quality and coverage of scanned and recorded images. In another aspect, embodiments described relate to reducing review time of scanned and recorded images from an imaging session or procedure.

### BACKGROUND

Medical imaging is typically referred to as Radiology because of the historical use of radiation-based imaging techniques to view internal structures of the human body. The origin of radiology is traditionally credited to Wilhem Rontgen, a German Physicist who discovered X-radiation (electromagnetic radiation in the 0.01 to 10 nanometers and with an energy levels ranging from 100 eV to 100 KeV) in 1895 as a result of his research on cathode ray tubes. Dr. Rontgen discovered that radiation emitted from the cathode ray tubes could pass through some forms of human tissue with varying degrees of absorption and that the X-radiation could expose photographic film. One of his first experiments was the now famous image of his wife's hand showing the bones of the hand with her wedding ring suspended as a halo around the proximal phalange of the third finger. The medical implications of viewing internal body structures were apparent and Dr. Rontgen was awarded the Nobel Prize for Physics in 1901.

Viewing the internal structures enabled radiologists to detect and diagnose conditions without the need for exploratory surgery, or before the conditions worsened and further compromised the patient's health. The applications of medical imaging have expanded as imaging technology has advanced. In addition to the singular X-ray presentations, multi-slice computed tomographic (CT) X-ray images are now standard tools for the radiologist. Imaging technologies that employ other energy sources, such as magnetic resonance imaging (MRI), radiation scintillation detection, ultrasound, and others have also expanded the radiologist's capabilities in diagnosing and detecting physiologic conditions.

For the advancement of these devices and methods to demonstrate utility for the medical imager, that is, for these new devices and/or methods to be adopted into the practice of radiology, they must demonstrate effectiveness and efficiency.

Effectiveness is the ability for the device or method to image internal structures and present the image viewer sufficient information on the internal structure to make a medical decision. If a radiologist wishes to examine the knee joint of a patient presenting with complaints of pain, the effective imaging device or method will be able to distinguish the internal structures of the knee in a way that will allow the radiologist to determine the nature of the complaint. If it is a fractured bone, the image must display, in some fashion, both the bone and the fracture. If it is a torn meniscus, the image must display, in some fashion, the bone structure with the attached meniscus, and the tear in the meniscus.

Efficiency is a measure of the resources required to perform an effective procedure. If a device or method can replicate the effectiveness of an existing device or method and, because of an advance in materials, manufacturing method, or other factors lower the cost of the device, then the decreased cost in performing the same function, or increase in efficiency, is a useful feature of the advancement. If a device or method can replicate the effectiveness of an existing device or method and, because of an advance in the functional design can reduce the overall time required to perform the procedure, or if that advancement can shift the time requirements away from more highly trained and skilled personnel to less highly trained and skilled personnel, then the resource shifting is an increase in efficiency which is a useful feature of the advancement.

Embodiments described herein provide for devices and methods for recording manually-obtained medical images so that they may be reviewed at a later time. The term “manual” is non-limiting and includes utilizing a device in which the image detection mechanism is designed to be used when held by the human hand. Some embodiments are directed to solving the problem of recording scans that adequately capture information needed for a physician or other trained reviewer to properly screen or diagnose a patient. For example, some embodiments provide for devices and methods for alerting an ultrasound operator if the distance between scanned images exceeds a maximum distance. In such cases, the operator will be alerted to rescan to ensure completeness of the imaging.

Further embodiments provide for effective and efficient devices and methods that allow the images recorded from a scan to be reviewed by a highly trained physician in an environment where he or she is not likely to be distracted by patient interaction or instrument adjustments, which improves the accuracy of the diagnostic and detection capabilities of the physician. Where an operator is not the ultimate reviewer of a scan, some embodiments described reduce the review time expended by reducing the number of images for review or the amount of time allocated for each image in the review. In such cases, these devices and methods allow the more highly trained image reviewer to be uncoupled from the time-consuming aspects of image acquisition and focus on the tasks associated with image interpretation and allows the operators to benefit from the reduction in time consumed by more highly skilled personnel.

There are many applications for medical imaging, but cancer screening and diagnoses are significant applications in the field. The clinical evidence is clear that early detection of cancerous lesions saves lives, and medical imaging is one of the foremost methods used to find cancerous lesions before the patient's condition becomes symptomatic. Embodiments described provide for devices and methods for recording and reviewing medical images for the purpose of diagnostic and screening image review. Applications of the described embodiments include use in screening and diagnosing many cancer types, such as cancer of the prostate, liver, pancreas, etc. Although the discussion below may reference breast cancer detection for describing embodiments and aspects of the invention, it should be understood; however, that the device has utility in the early discovery of other types of cancers and that omitting those cancers from this discussion does not limit the scope of the current invention. Moreover, the described embodiments are applicable to medical imaging in general and are not limited to any specific application provided as an example herein.

It is estimated that one out of eight women will face breast cancer at some point during her lifetime, and for women age 40-55, breast cancer is the leading cause of death. While methods for detecting and treating breast cancer initially were crude and unsophisticated, advanced instrumentation and procedures are now available which provide more positive outcomes for patients.

For instance, several studies have demonstrated that the ability to detect breast cancer tumors in advance of physical presentation (that is, before the discovery of a palpable lump or the appearance of a physical change in the breast's shape or appearance) has reduced breast cancer related mortality by as much as 30% (Tabar L, Vitak B, Chen H H, et al. The Swedish Two-County Trial twenty years later: updated mortality results and new insights from long-term follow-up. Radiol Clin North Am 2001; 38:625-51—IARC Working Group on the Evaluation of Cancer Prevention Strategies. Handbooks of Cancer Prevention, vol. 7, Breast Cancer Screening. Lyon, France: IARC Press, 2002.
Tabar L, Yen M F, Vitak B, Chen H H, Smith R A, Duffy S W. Mammography service screening and mortality in breast—Shapiro S, Venet W, Strax P, Venet L, Roeser R (1982) Ten to 14-year effect of screening on breast cancer mortality. J Natl Cancer Inst 69:349-355). Duffy demonstrated a clear correlation between the size of the cancer at the time of discovery and the survival rate (Stephen W. Duffy, MSc, CStat,* Laszlo Tabar, MD, Bedrich Vitak, MD, and Jand Warwick, PhD, “Tumor Size and Breast Cancer Detection: What Might Be the Effect of a Less Sensitive Screening Tool Than Mammography?” The Breast Journal, Volume 12 Suppl. 1, 2006 S91-S95)

Some of the reasons early detection leads to more positive outcomes is because that smaller tumor respond more positively to medical treatments, such as chemotherapy and radiation therapy and the smaller tumors are less likely to have metastasized to the lymph nodes and distant organ structures. In addition, smaller tumors are more easily excised in their entirety, reducing the probability of residual in-vivo cancer cells multiplying to the stage where metastasis can occur.

Advances in tumor detection procedures have radically changed the course of diagnosis and treatment for a tumor. With the advent of imaging devices, such as the mammogram, suspect tumor may be located when it is of relatively small size. Today, the standard of care in tumor detection generally involves both a mammogram and a physical examination, which takes into account a number of risk factors including family history and prior occurrences. Technical improvements in mammogram imaging include better visualization of the breast parenchyma with less exposure to radiation, improvements in film quality and processing, the introduction of digital technology, improved techniques for imaging, better guidelines for the diagnosis of cancer and greater availability of well-trained mammographers. With these advancements in imaging technology, a suspect tumor may be detected which is 15 mm or smaller This is compared with the 25 mm average size of a tumor which is discovered by physical palpation or other symptomatic presentation. More recently substantial progress has been witnessed in the technical disciplines of magnetic resonance imaging (MRI) and ultrasound imagining. These devices and methods have demonstrated the ability to reduce the average size at which cancers are detected. In the field of breast cancer screening, these reductions have been generally reduced to averages below 10 mm. With these advances, the location of a lesion is observable as diagnostic or therapeutic procedures are carried out.

Ultrasound has demonstrated particular utility in the detection of breast cancer for several reasons. Since the technology is an emission-reflection-detection technology rather than an emission-absorption-detection technology, as is the case of the mammogram, and since the sonic energy source transmits in multiple frequencies, each frequency interacting with the tissue differently, ultrasound is not as subject to shadowing phenomenon as is X-ray. Ultrasound is also one of the most prominent manual imaging technologies. That is, rather than the energy transmission and detection structures being mechanically fixed in place by other structure, the transmission and detection mechanisms are packaged in a single device which may be held in the human hand. The portability and small size of the device means that it can be used in locations, both geographic and anatomic, that are difficult for larger, more expensive imaging devices such as X-ray and MRI.

Because of ultrasound's superior capability, compared to mammography, in distinguishing between benign glandular tissue and malignant glandular tissue in the breast in women with a greater ratio of glandular tissue to fat (a condition termed “dense breasts”), ultrasound demonstrates a greater utility in cancer detection and diagnosis in these patents. Kolb (Kolb T M, Lichy J, Newhouse J H (1998) Occult cancer in women with dense breasts: detection with screening US—diagnostic yield and tumor characteristics. Radiology 207:191-199 and), Kaplan (Kaplan S S (2001) Clinical utility of bilateral whole-breast US in the evaluation of women with dense breast tissue. Radiology 221:641-649), Berg (Wendie A. Berg; Jeffrey D. Blume; Jean B. Cormack; et al., Mammography vs. Mammography Alone in Women at Combined Screening With Ultrasound and Elevated Risk of Breast Cancer, JAMA. 2008; 299(18):2151-2163 (doi:10.1001/jama.299.18.2151) and Kelly (Kevin M. Kelly,MD, Judy Dean, MD, W. Scott Comulada, Sung-Jae Lee, “Breast cancer detection using automated whole breast ultrasound and mammography in radiographically dense breasts”, Eur Radiol (2010) 20: 734-742) all demonstrated dramatic and significant increases in the number of cancers, with respect to mammography, in the population of women with dense breasts.

Medical imaging applications may be generally considered to fall in to one of three categories: (1) screening of asymptomatic patients, (2) diagnostic evaluation of symptomatic patients (i.e., those presenting symptoms discovered through the screening process, or outside of the screening process because they did not participate in a screening program or the screening program failed them), and (3) guidance for therapeutic procedures (i.e., those patients whose symptoms were confirmed, by the diagnostic testing process, to require some form of treatment). The clinical needs for each of these applications differ significantly, as do the needs, applications, and methods of the imaging techniques used in the three procedures.

In the diagnostic and guidance procedures, there is suspicion that a particular anomaly may be malignant and the status of that anomaly must be clarified (as is the case prior to a diagnostic procedure) or there is confirmation that an anomaly is malignant and that anomaly must be treated (as in the case of therapy). In both cases the ability to map the location of the anomaly is critical, but the ability to map the location of surrounding tissue is less critical. In both cases, there is positive identification of something abnormal in the patient's tissue and the subsequent actions are addressed to examining that abnormality, not to the normal surrounding tissue.

In the diagnostic examination the physician is already concerned with, and desires to characterize, a particular structure which has been previously characterized as “abnormal”. In the case of the suspected breast cancer the suspected abnormality is typically a result of a physical finding, such as the physical palpation of a lump in a particular location in the breast, a complaint of pain in a particular location in the breast, the appearance of some sort of deformity, such as skin thickening, skin distortion, abnormal nipple discharge, or the appearance of an abnormal structure on a screening imaging examination, such as a mammogram. Prior to the diagnostic examination it is typical that the region of interest is only identified as “suspicious”, not as a cancer. It is the purpose of the diagnostic examination to determine whether that “abnormal” region of interest is benign, malignant, or warrants further examinations to characterize more thoroughly. The position of the structure is known because it has been previously identified by one or more of a variety of methods described earlier. Therefore, the physician expects to find the abnormality.

In the diagnostic examination the physician is not concerned with structures other than the identified region of interest. In the example of breast cancer, the diagnostic examination is not only confined to the particular breast in which the abnormality was identified, but it is confined to the one particular quadrant of the particular breast in which the abnormality was found. There may be abnormalities in the other seven quadrants (there are four quadrants per breast). There may even be cancers in the other seven quadrants, but it is not the purpose of the diagnostic examination, however, to find those possible, but previously not identified, lesions. The purpose of the diagnostic examination is to characterize known lesions in known locations.

The screening examination differs from the diagnostic examination because (1) it is performed on an asymptomatic patient (that is, a patient who is considered healthy), so the physician expects all of the internal structures to be normal, and (2) it is performed on the entire structure, not just a localized area with a predetermined abnormality. As stated here, the physician expects normal tissue because the patient is asymptomatic, but he or she also expects normal tissue because the vast majority of patients have no abnormalities. In the case of breast cancer screening in the United States, only 3 to 5 patients per 1,000 screened have cancer. Only 1 in 10 have any tissue structures considered “not normal” enough to warrant further examination.

The contrast between screening and diagnostic can be exemplified in the mammography process. Since the expectation is that there is no cancer, there is no suggestion that a cancer is more likely to be in one quadrant rather than another. In the screening examination the Mammographer will compress the breast tissue between two paddles to pull as much of the breast as possible away from the chest wall to bring that tissue within the field of the X-ray source and X-ray detector. The X-ray source and X-ray detector are fixed in space and the patient tissue is immobilized within the field of exposure. The process requires significant patient manipulation and tissue distortion to pull the mammary tissue as far into the field of view of the X-ray radiation emitting and detecting imaging device as is possible. Since the X-ray radiation passes through the entire breast before exposing the detector, the image is a collection of “shadows” of structures within the breast and the entirety of the three-dimensional structure of the breast is reduced to a single two-dimensional image. The radiologist can tell with a single view whether the mammogram represents the entire breast.

In the diagnostic mammogram it is common for the mammographer to compress only portion of the breast which contains the region of interest. These “spot compressions” are often accompanied by magnification, with the result that only a portion of the breast appears in the image. Since the radiologist is not concerned with these other regions in the diagnostic examination, however, the tissue not presented by the image is of no concern.

Consistent with all of the descriptions of medical imaging devices is the concept of mapping the location of various tissue structures. The ability to map the images is critical because the device is not effective in practice if an abnormality is identified, but the physician does not know where it is within the patient's anatomy. Different portions of a three-dimensional object may be seen in different discreet images. The relative position of the slice is only known if the relative position of the patient to the imaging device is known when that image is obtained. Mapping can be as simple as identifying which limb was imaged by the X-ray, to acute, three-dimensional location of small structures in the complex structure of the complete anatomy.

It is not possible to “map” all of the structures a single two-dimensional view, however, because the human anatomy and human tissue structures are three dimensional. For example, if the X-ray reveals two shadows, or regions of interest, the device cannot determine which of two shadows is closest to the energy emitter and which is closer to the energy detector. A typical mammogram contains two images, each obtained by compressing the breast on planes that are not parallel, so that the location of the lesion can be determined through stereotactic calculations. Specifically, the location of a region of interest is typically described with regard to whether it is above or below the nipple, and whether it is medial or lateral to the nipple. For example, a lesion in the “upper-outer” quadrant is one that is located in the part of the breast which is nearest the shoulder and which presents lateral to the nipple (“outer”) on the cranio-caudad view and above the nipple (“upper”) on the medial-lateral-oblique view.

Another family of imaging devices maps the cellular tissue by taking more than one image on sequential parallel planes as a robotic element translates the imaging apparatus over the portion of the patient's anatomy which is to be studied. Each image is a slice, or cross-section of the region of cellular tissue that is to be imaged.

Computed Tomographic X-ray (CT) and Magnetic Resonance Imaging (MRI) image multiple “slices” or cross sections of the anatomy. Each slice, or frame, is a discreet image which describes all of the structures contained within that cross section, but do not describe information contained in adjacent slices. Computed Tomographic X-ray (CT) systems use a mechanism to move the X-ray source and detector over the entire body of the patient. Magnetic Resonance Imaging devices require the patient to lie, immobilized, in possibly in a prone position while he or she is literally moved, in totality, past the imaging structure. The rate of translation of that movement is controlled by a mechanical mechanism. Both of these devices use a form of robotics to control the translation of the imaging device to the patient, or the translation of the patient to the imaging device, so that each image may be mapped. The robotic control is designed to incorporate a real-time feedback mechanism to direct the path of the scanning and receiving mechanisms and direct the speed at which they scanning and receiving mechanisms translate. The goal of this real-time control is to assure that there is complete coverage (the path follows the directed course) and that the images are evenly spaced (to assure appropriate resolution). The primary purpose for controlling the speed is that most recording devices record at regular time intervals. A constant recording interval (e.g. frames/sec) divided by a constant translation speed (e.g. mm/sec) results in a regular spacing of images (e.g. frames/mm).

Unlike the robotic devices, the location of the manual imaging device is not controlled by an external mechanical structure when that device obtains the image. The device does not know where the imaging component is in space if the device does not know where the hand holding the device is in space. Therefore it does not know where the image is in space. One way that this problem has been addressed is to retrofit manual devices with location sensors that will provide spatial information of the images. For example, a manual scan to obtain regularly spaced images which cover the desired area is used to substitute the human operator for the robotic controls and use information from the location sensors to direct the human being, dynamically and in real time while he or she is scanning, to adjust the position, angle, and speed of the probe as it translates over the patient. If the user actually does respond to the prompts and adjusts his or her translational actions in real time, then the probe will translate over the skin at a constant speed and the images will be recorded at regular intervals. One drawback of this approach, however, is that there is no quality control to assure that the user responded to the prompts appropriately and that the images are actually being recorded at regular intervals. The situation is exacerbated if the program just assumes that the user made the adjustments and saves the images at the presumed locations and does not confirm actual spacing of the images. Another drawback of this approach is that it can be annoying to the operator to be prompted continually to adjust parameters on the scan. As such, there is a need for methods, devices, and systems that allow manual scanning without requiring that the operator scan the target area at a constant speed. Moreover, there is a need for systems and methods that interact with the operator to provide feedback either dynamically or non-dynamically during the scanning procedure that do not require the operator to alter scanning technique during the scan. Rather, the operator is provided feedback to repeat or rescan during the procedure but not necessarily during an actual scanning iteration.

Having the absolute mapping information of a discrete image is useful if that discrete image displays a particular region of interest. If the location of that particular region of interest is all that is required, then it is not necessary to know the relative position and orientation of each discrete image within the image set. If one wishes to reconstruct a three-dimensional map of a set of images, however, then the relative positioning information is critical. One discrete image may not be parallel to the orientation of the adjacent images or, for that matter, any of the images in the image set. The spacing between one discrete image and another may not be the same as the spacing between any other pair of discrete images within that image set. These disparities are of no consequence if the goal of the image procedure is merely to use the image information to map a region. One must merely determine the location of each pixel within all of the discrete images within the image set. These disparities are of consequence if one wishes to determine whether the quality of the map is adequate, in terms of coverage and resolution, as will be described later in this invention description.

Another factor to consider in the efficacy of any screening procedure is that of resolution, or the ability of the operator to resolve images of a desired size within the confines of the imaging technology. Most operators familiar with the art of image review are familiar with the concept of resolution when describing two-dimensional images, such as those presented on a television screen. For example, in the twentieth century standard television broadcasts presented images that were 704 by 480 pixels with a 4-to-3 aspect ratio (that is, the width of the screen is ⅓rd larger than the height), or sources of light, or pixels, displayed in an x-y grid. Each pixel is a single point which is uniform in color. If the television image was of a structure which was 70.4 cm by 48 cm is displayed on that 704 by 480 pixel screen, then each pixel describes a portion of that image which is 1 mm by 1 mm in size. Under these conditions, the ability of these images to distinguish, or “resolve”, smaller structures, such as a human hair (0.2 mm) is not possible. Zooming in on the image, as opposed to zooming in on the object with the camera, does not change the resolution. If one expanded one quarter of the screen to fit the size of the entire screen, then the entire screen would only contain 171 by 120 pixels of information. The display would be still be 704 by 480 pixels, but the expanded image would not contain more information and the single pixels of a single color that were in the smaller image would be presented as four adjacent pixels, each of the same color. In effect the individual small pixels would be replaced by larger “pixels”, but the resolution would not change by making that portion of the screen larger. Modern high definition (HD) Television presents images in a 1920 by 1080 pixel format. When one adjusts for changes in aspect ratios (16:9 instead of 4:3), the modern television image can resolve structures which are 2.5 times smaller than the 20th Century 704 by 480 pixel broadcast models. The modern high definition television could distinguish, or resolve, that human hair.

The ability to resolve smaller structures in the x-y presentation affects the operator's ability to interpret the two-dimensional image. Even when the resolution is sufficient to present small objects in some fashion, the operator may not be able to distinguish the exact nature of that small object unless the resolution can also present more details (that is smaller features) on the shape and texture of that object. Medical images typically have a broad range of resolution requirements and often those requirements are a function of the state of the technology. The earlier ultrasound devices packaged 64 imaging elements in a linear array and could not resolve features smaller than 2 mm. These devices found utility in a variety of medical imaging capacities. Modern ultrasound devices have 256 imaging elements and can easily resolve sub-millimeter features and the utility of the devices has expanded with the increased resolution capacity.

The level of resolution can vary along dimensional axes. For example, one manufacturer of a standard ultrasound system (the iU22, Philips Healthcare, Andover, Mass., USA), creates images from an ultrasound transducer with 256 active elements on an array which is 52 mm long. The system may be set to image variable depths of tissue. The design of the system allows it to produce more than one pixel per element and the image is displayed on a video monitor in a format which is 600 pixels by 400 pixels, with each pixel representing a unique tissue structure in the space of the plane of the image. Thus, an ultrasound image acquired from this system, with a depth setting of 5 cm, would have a resolution of 11.5 pixel/mm in the horizontal, or X axis and 8.0 pixel/mm in depth, or the Y axis. Changing the depth setting to 4 cm would change the Y pixel resolution of 10.0 pixel/mm (the X pixel density would remain unchanged).

In three-dimensional imaging, the translational resolution can differ greatly from the resolution presented in the planar presentation of each discrete image. Even if the resolution of the X-Y presentation of any one discrete image is sufficient to distinguish 1 mm structures, it is possible for a 1 mm structure to be missed entirely if the space, or “Z” vector, between the discrete images is greater than 1 mm. If one assumes a spherical region of interest and if the required Z-spacing vector spacing is a function of the X-Y resolution of the imaging device, then with most modern imaging devices, if the spacing between discrete images is less than ½ of the size of the minimum requirement for detection of regions of interest, then it is reasonable to assume that at least one discrete image will present a cross section of the lesion with a size which is large enough to be resolved on the X-Y presentation of that discrete image. By the way of example, if the operator desires to view a 1 mm region of interest, and spacing between discrete images is greater 0.5 mm, the smallest cross-sectional presentation of that 1 mm region of interest will be 0.86 mm. If the X-Y resolution of the images is smaller than 0.86 mm, as it is with most modern hand-held imaging devices (such as ultrasound), then the intra-image resolution is sufficient. The early CT devices had 8 discreet images. Although any single X-Y slice could resolve lesions as small as a millimeter, the inter-slice spacing made resolution of lesions smaller than 8.6 mm unreliable. Modern 64-slice CT devices have a 0.5 mm inter-slice spacing, making the ability to diagnose millimeter sized lesions possible.

As used herein, in some embodiments, the individual image slices are referred to as “discrete images” while the set of discrete images obtained in a single scan sequence are referred to as a “set of discrete images” or a “scan track”. Moreover, “scan” or “scan sequence” or “scan path” or “set of discrete images” are used in some embodiments to refer to a plurality of images recorded sequentially as the hand-held imaging probe is placed in contact with the patient and is moved from one location to another location on the patient.

A clear understanding of absolute and relative coordinate geometries is essential when mapping tissue images and determining resolution. Since the discrete images are typically presented in a two-dimensional format, whether on paper or on a video screen, mapping of that format is typically presented in a means compatible with the X and Y axes of a Cartesian coordinate system. For example, previously described Philips ultrasound device displays the images on a video monitor in a format which is 600 pixels by 400 pixels. Thus, an ultrasound image acquired from this system (which has a probe width of 5.2 cm), with a depth setting of 5 cm, would be 0.087 mm/pixel in the X axis and 0.125 mm/pixel in the Y axis.

A second image in the sequence would also represent a tissue slice that is 5.2 cm by 5 cm. The corresponding pixels are the pixels which are at the same X-Y coordinate in both images. The X-Y location of the first pixel of the first row of one image corresponds to the X-Y location of the first pixel of the first row of the second image; the X-Y location of the second pixel of the first row corresponds to the X-Y location of the second pixel of the first row, and so forth until the last X-Y location of the pixel of the last row of the first image, which corresponds to the X-Y location of the last pixel of the last row of the second image.

Hand-held imaging devices rely on a human operator to translate the imaging probe over the tissue to be examined and present resolution challenges that are very different from the robotic devices. The X-Y resolution of a single image may be comparable to another method. For example, the pixel spacing in modern ultrasound systems is 0.125 mm, approximately the same as a mammogram. The primary challenges in the efficacy of a hand-held device are the ability to map individual images, the ability to resolve between the discrete images in the image set, and to determine whether the family of image sets represents complete coverage of the structure.

As was described earlier, screening examinations require that the user image “all” of the tissue. Seeing “all” of the tissue is more a function of coverage than it is of resolution. Coverage, or field of view, is a description of the extent of the field of imaging, not the quality of the imaging. An X-ray of the kidney which images only half of the kidney may have finely detailed resolution, but it does not cover the entire kidney. Conversely, a blurry mammogram of the entire breast “covers” the entire breast, but may not do so with adequate resolution to be a useful examination.

As used herein, the term “coverage” is not intended to be limited to any particular meaning. The term broadly includes, at least, the distance, surface, volume, area, etc. that is imaged during a medical imaging session. For example, determining coverage of a scan would include evaluating whether there are any gaps in the relative positions of the images contained in (between) two or more scan track sets (e.g. scan-to-scan spacing or distance). As a comparison, resolution describes at least the X-Y and x-y-z resolution of each individual image and the relative spacing of the discrete images within a single scan track (e.g., image-to-image spacing or distance).

With an X-Ray or MRI or CT scan a single image, or slide, will tend to cover all of the tissue in a cross-section that can be 30 cm in size or larger. However, a typical ultrasound probe is 4 cm to 6 cm in size. It would require five or more parallel scan track sets of a 6 cm ultrasound probe to encompass the same volume of tissue that could be imaged with a single 30 cm mammogram.

Robotic devices have been used to previously achieve coverage because the desired field of view is predetermined and the systems are able to calculate the appropriate translational scan paths to encompass that field of view and they are programmed to translate the energy scanning and receiving elements along the predetermined paths. In contrast, manual imaging devices are operated based on the technical experience and subjective judgment of the human operator. The quality, particularly coverage, of the scanned recorded images varies widely depending on the operator. For example, if the operator scans too quickly, the images in a scan sequence may be spaced too far apart to show a potential cancerous region. Similarly, if the operator spaces two scan sequences too far apart, then there may be areas between scan rows that have not been scanned for review. As such, some embodiments described provide methods, devices, and systems for recording images to ensure that recorded images during a manual scanning session have adequate coverage.

As used herein, a “scan track,” in some embodiments, refers to any set of discrete images recorded by a medical imaging method, device, or system. The set of discrete images can be obtained by any method or device. In some cases the set of discrete images are obtained when an operator (1) places the probe on the patient, (2) begins recording images, (3) translates the probe across the surface of the skin, (4) stops recording the images. In other embodiments, a scan track is a set of sequential discrete images with unique relative spacing between individual discrete images. In such cases, the set of discrete images can encompass a volume which is as wide as the imaging probe design allows, as deep into the tissue as the imaging probe allows, and as long as may be accomplished by the act of recording the images while translating the probe across the skin.

Another difference between traditional mammography or the robotic devices and traditional hand-held imaging technologies is that mammography and the robotic devices depend on separating the imaging process in to two steps, (1) recording the image and (2) reviewing the image. With the hand-held devices the images can be presented in real-time, so the reviewer can dynamically review structures. When performing the procedure in real time, the skilled operator may believe that he or she is skilled in appropriately translating the probe to cover the breast entirely and to translate the probe with appropriate speed, and may believe that he or she does not need real-time feedback to achieve these goals. When the real-time images are recorded by one operator for later review by another, as is necessary to address the time constraints associated with screening, the reviewer does not have the ability to confirm the location of the image nor does he or she have the ability to confirm the spacing between adjacent images, if appropriate. The reviewer does not have the ability to determine the resolution in the “z” plane. Since the reviewer does not know the relative position of each scan track set of discrete images, the reviewer does not have a concept regarding whether this family of sets represents complete coverage.

For the purpose of this discussion, assume that X and Y axes of a Cartesian coordinate system are used to define a two-dimensional array of ultrasound scanning derived images containing a multiplicity of pixels, where the term pixel refers to the basic unit of a video screen image and can be defined by its X and Y coordinate value in any predetermined reference frame defining the location of zero for both the X and Y coordinates. These two-dimensional ultrasound images are generated by an ultrasound probe comprising a linear scanning array. A modern high-end scanning array consists of 256 transmitting and receiving transducers packaged in an ultrasound probe, said linear array of transducers having a width of 38 mm to 60 mm. These linear arrays of transducers produce images with the spacing between adjacent pixels ranging from 0.06 mm to 1 mm. Each individual pixel within the ultrasound-derived planar image is defined by a unique X and Y coordinate value. The two-dimensional resolution, or two-dimensional density of the pixels within each ultrasound scan-derived two-dimensional image (i.e., number of pixels per square centimeter of the image) is constant and is a function of the ultrasound system hardware and remains the same for each adjacent image in the scan process. This resolution allows routine identification of tissue abnormalities (e.g., cancers) as small as 1 mm to 5 mm.

The primary challenges in the three-dimensional reconstruction are the spacing between adjacent pixels in the third axis of the XYZ Cartesian coordinate system, viz., the Z-axis and the relative location of the families of sets of discrete images obtained during the scanning process.

The spacing along the Z-axis is dependent, in part, on the rate of change of the position and angle of the ultrasound probe between the creation of any two sequential and adjacent two-dimensional images. The change in the spacing between two sequential two-dimensional images depends on five factors:

One factor is the rate at which the ultrasound system hardware and software are capable of processing the reflected ultrasound signals and constructing the two-dimensional images (i.e., number of completed two-dimensional ultrasound scans per second).

The second factor is the rate at which the displayed images can be recorded, for example by a digital frame-grabber card. By way of example, if the ultrasound system displays 10 discrete images per second and a frame-grabber card can record 20 frames per second, then the recorded set of images will have 20 images but will, in reality, have only 10 discrete images with each image having a replicate. By way of another example, if the ultrasound system displays 40 frames per second and the frame grabber records 20 frames per second, the recorded set of images will have 20 discrete images, but will not have recorded an additional 20 discrete images.

A third factor is the rate at which the ultrasound probe is translated along the scanned path. By way of example, the faster the operator moves the ultrasound probe, the greater the spacing will be in the Z direction and/or the slower the combined rate at which the ultrasound system hardware and software are capable of processing the reflected ultrasound signals and constructing the two-dimensional images and the image recording hardware can store the processed images (i.e., the lower the rate of completed two-dimensional ultrasound scans recorded and stored per second), the greater the spacing will be in the Z direction. Conversely, if the operator moves the ultrasound probe more slowly, the smaller the spacing will be in the Z direction.

The fourth factor is the relative orientation of the hand-held probe during the scanning process. Because the probe is not held rigid by a mechanical mechanism, the translational distance between adjacent frames is not a constant. For example, if the discrete images within an image set were perfectly parallel, then the Z spacing between corresponding pixels would be the same for each pair of corresponding pixels in two discrete images. If the probe were rotated along the lateral axis (pivoted, or pitch) then the Z spacing of the corresponding pixels at the top of a pair of images would vary from the Z spacing of the corresponding pixels at the bottom of a pair of images. If the probe were rotated along its longitudinal axis (roll) then the Z spacing of corresponding pixels on the left side of the a pair of images would vary from the Z spacing of the corresponding pixels on the right side of the pair of images.

The fifth factor is associated with the rotation of the probe along its vertical axis (yaw). The distance between two corresponding pixels in a pair of images differs if the two images are recorded when rotation on the vertical axis differs.

In addition to determining the spacing between discrete images within a scan track set, it is important to understand the relative relationship between separate scan track sets within a family of scan track sets which describe a complete scan. This variable is an important factor in the function of coverage. If the images obtained within a single scan track adequately cover the tissue, then there is no need for a second scan track. If the single scan track is too small, in width or length, to cover the entire tissue structure, then a second scan track is needed. Since each scan track has its own set of discrete images, and since each discrete image has its own mapping location coordinates, it is possible to determine whether two separate scan tracks represent the exact same region of tissue, adjacent regions of tissue with some overlap, adjacent regions of tissue with no overlap, adjacent regions of tissue with some gap in between, or regions of tissue with no anatomic relation to each other.

The reconstruction of a plurality of scan tracks can describe a covered region if the scan tracks between any two adjacent scan tracks can be reconstructed to form a contiguous region of images with no gaps in coverage and if the extent of the reconstruction encompasses the entire tissue structure to be imaged.

As described earlier, prior techniques have relied on robotic machinery to calculate the number, the direction, and extent (length) of scan tracks required to have complete coverage and control the scanning variables ((1) image refresh rate, (2) image recording rate), (3) the translational speed of the probe, (4) the rotation of the probe along the lateral and longitudinal axes, and (5) and the rotation of the probe along the vertical axis) so that the resulting family of scan tracks contains images which have the coverage and resolution required for a “complete” examination of the tissue.

Robotic approaches to ultrasound imaging require the use of expensive mechanical equipment that is also subject to regular service and calibration to assure that the machine driven ultrasound probe is in the assumed position and computed orientation as required to assure that a complete and systematic diagnostic ultrasonic scan of the target living tissue has been actually achieved.

An objective of the present invention is to enable and assure the completeness of an ultrasound diagnostic scan of the target tissue (e.g., human breast), in terms of area covered and resolution of the relative spacing of the images within that area covered, without the need for robotic mechanical systems for the support, translation and computed orientation control of an ultrasound probe. Some embodiments enable the use of hand-held diagnostic ultrasound probe scanning methods while assuring that a complete scan of the targeted tissue is achieved.

As important as the imaging requirements are to achieving a practical screening technology, time constraints can also affect practicality, thus the utility, of the device. Berg et al., describe that the average time to perform a manual ultrasound screening examination of both breasts is 19 min and the median time is 20 minutes (Wendie A. Berg; Jeffrey D. Blume; Jean B. Cormack; et al., Mammography vs. Mammography Alone in Women at Combined Screening With Ultrasound and Elevated Risk of Breast Cancer, JAMA. 2008; 299(18):2151-2163 (doi:10.1001/jama.299.18.2151). This time does not consider the time it takes the radiologist to walk from the reading room to the ultrasound examination room, the time it takes to interact with the patient, or the time it takes to return to the reading room from the ultrasound examination room.

The time required to view the actual images is much shorter. By the way of example, a standard screening ultrasound examination involves 2,000 to 5,000 images, obtained in a series of rows scanned according to one of many scan disciplines. If the recorded images are reconstructed and viewed as a cine, that is the sequential display of a set of discrete images, as in a movie, so that the viewing experience is the same as the operator would have experienced had he or she been performing the hand-held procedure in real time, then the review time could be as short as 200 seconds (less than 4 minutes). The concept of the cine presentation goes back more than a century, to Edison, but Freeland describes the use of the cine viewing technique for the review of ultrasound images in 1992 (U.S. Pat. No. 5,152,290).

It is standard practice for trained radiology technologists to perform the imaging function for most radiology procedures. The technologist's duties are to obtain good quality images and present them to the radiologist to interpret. In the way of an example, the average time required to obtain and record a standard 4-view mammogram is 10 min to 15 min, but the radiologist can interpret those images in less than two minutes.

As described earlier, although it is not possible for a skilled and trained operator to objectively determine the completeness of the area covered, and the resolution (in terms of the relative spacing between adjacent images) of a scan when they are personally performing a manual examination, they may believe, subjectively, that the coverage and resolution are adequate. If the reviewer is observing a set of images that were recorded by another operator, however, it is not possible for the reviewer to have any defendable means of determining whether the area covered represents the entire structure or that the resolution, in terms of spacing between images, meets the minimal standards that the user requires. Mapping the images and calculating the resolution and coverage of the resultant sets of images, as described in some embodiments herein, allows the ability to divide the imaging and reviewing tasks and, thus, allows the time savings associated with performing the procedure in a manner where it is recorded by one individual and reviewed by another and still provide some level of confidence as to the aforementioned resolution and coverage.

Mapping the images for resolution and coverage allows the cine review process to be speeded up as well. Speeding up the review reduces the requirements in the radiologist's time, providing utility to the operator. Standard cine review presents a series of discrete images in quick succession, but at a constant time interval (frames per second, or fps) with a dwell time for each frame a function of that time interval. By way of example, if the desired frame-to-frame resolution in an examination is 1 mm, and images are recorded at exact 1 mm intervals, and if the frames are reviewed at 10 fps, with a frame dwell time of 0.1 sec/frame, then the time to review a 10 cm scan track of discrete images (100 images) would be 10 seconds. If the images are recorded at exact 0.1 mm intervals (1,000 images) the review time would be 100 seconds. Although there is additional information in those 900 additional images, the incremental improvement in patient care may not be warranted for the additional 1.5 minutes of physician time to review the track. If one considers that there may be as many as 16 such scan tracks for each breast, then the time differential could be 320 seconds (just over six minutes) vs. 3,200 seconds (just over one hour).

Some embodiments described provide for systems and methods for providing a speeded review time by varying the dwell time between successive discrete images and calculating that dwell time as a function of the distance between adjacent images. The resultant presentation would be provided in distance covered per second (dcps) not frames per second. By way of example, if the system recorded 19 images, with the Z-plane location of those images being 0.0 mm, 0.7 mm, 0.9 mm, 1.9 mm, 2.5 mm, 2.8 mm, 3.6 mm, 3.7 mm, 4.0 mm, 4.7 mm, 5.1 mm, 5.6 mm, 6.6 mm, 7.0 mm, 7.6 mm, 8.2 mm, 8.5 mm, 9.5 mm, and 10.0 mm, then the review time for those 19 images at 10 fps (that is a dwell time of 0.1 sec/frame) would be 1.8 sec. If individual dwell times were assigned unique values with criteria based on amount of tissue to be imaged per second and the spacing between discrete images, then the review time could be shortened considerably. By way of example, if the dwell times of the 19 images described earlier were changed to 0.07 sec, 0.02 sec, 0.1 sec, 0.06 sec, 0.03 sec, 0.08 sec, 0.01 sec, 0.03 sec, 0.07 sec, 0.04 sec, 0.05 sec, 0.1 sec, 0.04 sec, 0.06 sec, 0.06 sec, 0.03 sec, 0.1 sec, and 0.05 sec, respectively, then the review time would be 1.00 seconds.

Some embodiments also provide for a means of speeding the review time by displaying only those images which provide incremental information that the operator deems useful. By way of example, if the user chooses an optimal resolution of 1.0 mm between images, and if there is more than one image in that 1.0 mm spacing, then the extra images are redundant. The system and method may choose to not display the redundant images. By further way of example with the images described in the previous paragraph, if the operator chooses an optimal image spacing of 1.0 mm, then the system would only display those images recorded at 0.0 mm, 0.9 mm, 1.9 mm, 2.8 mm, 3.7 mm, 4.7 mm, 5.6 mm, 6.6 mm, 7.6 mm, 8.5 mm, 9.5 mm and 10.0 mm. The images recorded at 0.7 mm, 2.5 mm, 3.7 mm, 4.0 mm, 5.1 mm, 7.0 mm, and 8.2 mm would be culled. If the retained images were displayed at 10 fps (a dwell time of 0.1 seconds/frame) then the image review time would be 1.1 seconds, not the 1.8 seconds that would be required if all of the images were reviewed.

Another system and method for reducing the review time required by the radiologist would be to cull images whose information is contained completely within another set of discrete images. By way of example, if the operator is reviewing a scan of the breast which contains 12 sets of discrete images, each image originating at the nipple and extending radially to the base of the breast at each of the 12 clock positions, there will be images within some of those sets of discrete scans that image tissue structures that overlap or are partially or completely imaged by other images or groups of images. By way of example, if because the radius of coverage decreases as the scans get closer to the nipple, the 5 mm probe extends from 10 o'clock to 2 o'clock when the probe is performing the 12 o'clock scan is only 1 cm from the nipple, and the probe extends from 1 o'clock to 5 o'clock when the probe performing the 3 o'clock scan is just 5 mm from the nipple, then there is a substantial and possibly complete overlap between these two scans and the images recorded by the 1 o'clock scan at 5 mm from the nipple and the 2 o'clock scan at 5 mm from the nipple contain redundant information. If those images were removed from the review set then the result would be a time savings. This system and method teaches a means of distinguishing which images contain information that is completely or partially contained in one or more images from other sets of discrete images in the scan and removing those images from the review set. Overlap of information in images could be anywhere from about 10% to about 100%. In some embodiments, images with information having 80%-100% overlap with other images are removed from the review image set.

### SUMMARY OF THE DISCLOSURE

Some embodiments described provide for methods, apparatus and systems for determining the resolution or spacing of the image-to-image spacing of discrete images within sets of discrete images, or scan sequences, and determining the coverage of multiple sets of discrete images, or scan sequences, in a hand-held imaging scan of targeted human tissue such as the human breast. In one embodiment, the range of the image-to-image resolution within each scan sequence is about 0.01 mm to 10.0 mm. In another embodiment, the image-to-image resolution within each scan sequence is about 0.1 mm to 0.4 mm. In further embodiments, the image-to-image resolution within each scan sequence is about 0.5 mm to 2.0 mm.

In another embodiment, the range of the image-to-image resolution within each scan sequence is a pixel density between 9,000 and 180,000,000 pixels/cm3. In other embodiments, the pixel density is between 22,500 and 18,000,000 pixels/cm3. In further embodiments, the pixel density is between 45,000 and 3,550,000 pixels/cm3.

In some embodiments, the range of coverage, in terms of the overlap of the border of adjacent scan tracks is between about −50.0 mm to +50.0 mm (where a negative overlap value indicates a positive gap value, or spacing between the borders of adjacent scan tracks). In other embodiments, the overlap of the border of adjacent scan tracks is between about −25.0 mm to +25.0 mm (where a negative overlap value indicates a positive gap value, or spacing between the borders of adjacent scan tracks). In further embodiments, the overlap of the border of adjacent scan tracks is about −10.0 mm to +10.0 mm (where a negative overlap value indicates a positive gap value, or spacing between the borders of adjacent scan tracks).

Examples of hand-held imaging procedures include, but are not restricted to, ultrasound examinations. Objective determination that user-defined levels of coverage and resolution are achieved is critical, particularly when one clinical practitioner performs the recording function during the hand-held scan and another practitioner, who was not present at the recording procedure, reviews those pre-recorded images. Objective determination of coverage and image-to-image resolution or spacing that the subsequent review of the recorded images by a trained clinical specialist following the scanning procedure is critical to assure that the subsequent review does not result in a false negative assessment due to the fact that some regions of the targeted tissue volume were inadvertently omitted. Such omissions can be caused by the inadvertent excessive spacing between successive hand-held scans that are intended to cover the tissue structure, excessive image-to-image spacing within a single hand-held scan that can result from variations in rate of translation of the hand-held imaging probe and/or the excessive rate of change of the orientation of a hand-held imaging probe during the scanning of a targeted tissue volume such as the human breast.

The tracking of the position and computed orientation of a hand-held imaging probe can be accomplished by affixing position sensors on the body of the ultrasound probe at predetermined locations relative to the design geometry of the hand-held imaging probe imaging elements. Three or more sensors are affixed to the hand-held imaging probe to enable the computation of the position (viz., x, y, z coordinates) of the hand-held imaging probe imaging elements and the computation of the orientation of the longitudinal axis of the hand-held imaging probe body. Said orientation coincides with the axis of image, for example the planar ultrasound beam emitted into the tissue being interrogated.

According to some embodiments, the accurate and dynamic computation of the position of the hand-held imaging probe's imaging elements enables the determination of the actual spatial position and computed orientation of manually scanned, sequential pathways completed along the tissue surface. The computed position and computed orientation of each manually scanned, sequential pathway, combined with information regarding the dimensional size of each recorded image, along the tissue surface enables the further computation of the physical spacing or distance between scan sequences. This computation can be rapidly completed during the course of the manual scanning process or procedure and a visual and optional audible cue as well as an image is provided showing the paths of completed scan sequences to identify where re-scanning is required. This intra-procedure computation of the distances between adjacent scan sequences determines whether complete coverage of the targeted tissue volume is achieved with the hand-held imaging probe. Accordingly, this intra-procedure computation of the distances between adjacent scan sequences assures that the completed scan sequences cover the targeted tissue structure by assuring that the individual scan sequences overlap, or are separated by an acceptable distance.

In addition, according to the teachings of this invention, the accurate and dynamic computation of the position of the hand-held imaging probe's imaging elements enables the determination of the actual spatial position and computed orientation of each image within the sequential and manually scanned pathways completed along the tissue surface of the targeted defined volume of tissue. The physical spacing between discrete images in scanned pathways can be determined by using the computed position and computed orientation of each manually scanned, sequential pathway with information regarding the dimensional size of each recorded image. This computation can be rapidly completed during the course of the manual scanning process and a visual and optional audible cue as well as an image is provided showing the paths of completed scan sequences to identify where re-scanning is required. This intra-procedure computation of the distances between adjacent scan sequences determines whether image-to-image resolution of the targeted tissue region is achieved with the hand-held imaging probe is achieved by identifying distances between completed discrete scan images that are inadvertently separated by an unacceptably large distance.

In addition, according to some embodiments, the accurate and dynamic computation of the orientation (based on the positions of the three or more sensors) of the hand-held imaging probe's longitudinal axis (hence, the orientation of its emitted planar imaging beam) enables the computation of image-to-image resolution or spacing by enabling the computation of a chord length between the planar images at the maximum depth of tissue being scanned for any two successive time steps at which images are obtained and recorded during any manual scan sequence along the tissue surface. The computed rate of change of orientation of the hand-held imaging probe (derived from position sensors affixed to the hand-held imaging device) during a manual scan sequence along the tissue surface enables the further computation of the physical spacing (i.e., chord length) between planar ultrasound scans between two successive time steps during a scan sequence. This intra-procedure computation of the chord distances between hand-held imaging planar scans acquired and recorded for any two consecutive time steps assures that a complete hand-held imaging scan of the targeted tissue region is achieved in terms of image-to-image resolution or spacing. This is accomplished through position change computations, thereby identifying any completed scan sequence in which the chord distances, at the maximum depth of interrogation, between adjacent discrete images are unacceptably large.

In addition, according to some embodiments, the accurate and dynamic computation of the orientation (based on the positions of the three or more sensors) of the hand-held imaging probe's lateral axis (hence, the orientation of its emitted planar imaging beam) enables the computation of image-to-image resolution by enabling the computation of a chord length between the sides of two planar images, from the surface of the tissue to the maximum depth of tissue being scanned for any two successive time steps at which images are obtained and recorded during any manual scan sequence along the tissue surface. The computed rate of change of orientation of the hand-held imaging probe (derived from position sensors affixed to the hand-held imaging device) during a manual scan sequence along the tissue surface enables the further computation of the physical spacing (i.e., chord length) between planar ultrasound scans between two successive time steps during a scan sequence. This intra-procedure computation of the chord distances between hand-held imaging planar scans acquired and recorded for any two consecutive time steps assures that a complete hand-held imaging scan of the targeted tissue region is achieved in terms of image-to-image resolution. This is accomplished through position change computations, thereby identifying any completed scan sequence in which the chord distances, at the maximum depth of interrogation, between adjacent discrete images are unacceptably large.

An alternative method for assuring the completeness of any individual scan sequence, in terms image-to-image resolution/spacing, (e.g., any individual path scanned beginning at the nipple of the breast and ending at the chest surface beyond the perimeter of the breast boundary) involves computation of the pixel density in each unit volume within the swept volume of the scan sequence. In the case of an ultrasound examination of the breast, the swept volume of the scan sequence would be the volume defined by (a) the width of the ultrasound beam, which is defined by the length of the ultrasound transducer array (e.g., 5 cm), (b) the depth of recorded penetration of the ultrasound beam into the targeted living tissue (e.g., 5 cm) and (c) the total length traversed in the individual scan sequence (e.g., 15 cm). This total volume (375 cubic cm in the present example is then subdivided into unit volumes (e.g., cubical volume of dimensions 1.0 cm×1.0 cm×1.0 cm). For this example, the swept volume would be subdivided in to 375 unit volumes. The number of ultrasound pixels within that unit volume would be the total number of pixels in the portion of each discrete ultrasound image which is defined as being within the three-dimensional boundaries of the unit volume. The number of ultrasound scan pixels contained in each unit volume is computed and this number is compared to a predetermined Minimum Pixel Density number. If the computed pixel density within any unit volume (i.e., any of the 375 unit volumes in this example) within the swept volume is less than the Minimum Pixel Density, then the operator is alerted at the end of the scan sequence that scan sequence just completed is incomplete and that it must be repeated including a display of instructions to improve scanning method (e.g., reduce scanning speed and/or rate of change of orientation of hand-held ultrasound probe during the repeated scan sequence).

In addition to affixing spatially arranged position sensors on a hand-held and manually applied imaging probe, another embodiment also provides a receiving device to detect and digitally record and store a digitized set of numbers which indicate the position and computed orientation of the hand-held imaging probe as well as the time associated with said position and computed orientation at each time step (i.e., time-stamped position and computed orientation data). Also, a digital data storage device provides for the recording of hand-held imaging image data at multiple times per second, images which are also time stamped for purposes of subsequent review by an individual or software capable of expert analysis of hand-held imaging images to detect the presence of suspicious lesions within the targeted tissue volume.

Once the completeness of the hand-held imaging scan has been confirmed (and scan sequences repeated if any regions within the targeted tissue volume were not scanned), the complete set of consecutive hand-held imaging images can be reviewed by play back of the recorded images at regular time steps (e.g., 6 to 12 frames per second).

According to one aspect of the present invention there is provided an imaging system for acquiring a sequence of two-dimensional images of a target volume represented by an array of pixels I (x,y,z) comprising [a] a hand-held imaging probe to scan said target volume along a path, which may be predetermined or may be determined dynamically as the operator performs the procedure, and generate a sequence of digitized two-dimensional images thereof representing cross-sections of said target volume on a plurality of planes spaced along said scanning path; said scanning path may any geometric path determined by the scanning personnel and is not required to be linear; [b] a data storage medium for storage of digital data associated with each pixel of each two dimensional image in a sequence of digitized two-dimensional images together with other related image data defining the location of said two-dimensional images in said memory and defining interpretation information relating to the relative position of pixels within said two-dimensional images and to the relative position of pixels in adjacent two-dimensional images within said target volume; and [c] software algorithm to determine if the relative position of pixels in adjacent two-dimensional images within said target volume exceeds a predetermined limit.

According to another aspect of the present invention there is provided an imaging system for acquiring two or more sequences of two-dimensional images of a target volume represented by an array of pixels I (x,y,z) comprising [a] a hand-held imaging probe to scan said target volume along two or more scanning paths, which may be predetermined or may be determined dynamically as the operator performs the procedure, and generate two or more sequences of digitized two-dimensional images thereof representing cross-sections of said target volume on a plurality of planes spaced along said scanning path; said scanning paths may any geometric path determined by the scanning personnel and is not required to be linear; [b] a data storage medium for storage of digital data associated with said sequences of digitized two-dimensional images together with other related image data defining the location of said two-dimensional images in said data storage medium and spatial and temporal information relating to the relative position of pixels at the edge of said two-dimensional images and to the relative position of pixels in one or more adjacent two-dimensional images at the edge of the adjacent scan sequence; and [c] software algorithm to determine if the relative position of pixels in adjacent two-dimensional images within said target volume exceeds a predetermined limit.

According to yet another aspect of the present invention there is provided an imaging system for acquiring two or more sequences of two-dimensional images of a target volume represented by an array of pixels I (x,y,z): [a] a hand-held imaging probe to scan said target volume along two or more scanning paths, which may be predetermined or may be determined dynamically as the operator performs the procedure, and generate two or more sequences of digitized two-dimensional images thereof representing cross-sections of said target volume on a plurality of planes spaced along said scanning path; said scanning paths may any geometric path determined by the scanning personnel and is not required to be linear; [b] a data storage medium for storage of digital data associated with each pixel of said sequences of digitized two-dimensional images together with other related image data defining the location of said two-dimensional images in said data storage medium and constructing a three-dimensional array of said pixel locations; and [c] software algorithm to determine if the pixel density within a predetermined volume is greater than a predetermined limit.

Another embodiment of the present invention incorporates methods, apparatus and system for optical recognition (e.g., using infrared wavelength detection of unique markers affixed to hand-held imaging probe assembly) to continuously detect the position and orientation of a hand-held ultrasound probe assembly in place of the use of electromagnetic radiofrequency position sensors. In some embodiments, an optical recognition based position and orientation detection method, apparatus and system accurately determines the position of each two-dimensional ultrasound scan image and, thereby, the temporal and spatial position of each pixel within each two-dimensional ultrasound scan image.

Another embodiment of the present invention incorporates methods, apparatus, and system for optimizing image review time on the part of the physician. The recorded images are reviewed as a series of still images, those images being presented for a fixed period of time (e.g. 0.1 sec each). The more images there are to review, the longer the review time for the physician will be. Since optimizing (that is, reducing) review time is an important aspect of any image review procedure, care must be taken that the review is thorough, but not excessive. Since the images will be recorded with a hand-held probe, it is possible that the relative spacing of adjacent images will vary. Some images may be spaced so closely that they are, in effect, redundant, while others may be spaced so far apart that it is possible to miss important structures. The prior part of this application describes methods for dealing with the latter scenario. Some embodiments described will optimize physician review time by one of two methods:

1. The system will choose an optimal image spacing parameter and a maximum allowable image spacing parameter. The maximum spacing between relative images will be calculated and the images for which the relative spacing is closest to the optimal spacing parameter shall be saved, and intermediate images shall be culled. For example, if the operator varies his or her scan so that images are recorded at 0.0 mm, 1.0 mm, 1.5 mm, 2.0 mm, 2.8 mm, 3.0 mm, 3.2 mm, 3.5 mm, 3.7 mm, 4.0 mm, 4.3 mm, 4.7 mm, 5.0 mm, 5.5 mm, and 6.0 mm, and the review time is 0.1 sec per image, the time to review these images is 1.5 seconds. If the operator decides that the optimal spacing to detect small lesions is 1.0 mm, then those images that were recorded at 1.5 mm, 2.8 mm, 3.2 mm, 3.5 mm, 3.7 mm, 4.3 mm, 4.7 mm, and 5.5 mm are not necessary to find the small lesions. They are redundant and add 0.8 seconds to the review time. Image review time could be halved, from 1.5 seconds to 0.7 seconds, by culling these images (FIG. 1). Review time can be reduced significantly for a patient during an ultrasound reading procedure. For example, the review time may be reduced by more than half—e.g., 15 minutes to 7 minutes.

2. The system will vary its playback time based on the spacing of the images. Computers and computer display systems make it relatively simple to vary the dwell time for displayed images when replaying them. In the example cited above the first image (0.0 mm) could be displayed for 0.1 seconds while the four subsequent images (1.0 mm, 1.5 mm, 2.0 mm, and 2.8 mm) could be displayed for 0.05 seconds, and the time to review images covering the region would be 0.3 seconds. If, in this example, the dwell times for the images recorded at 3.2 mm, 3.5 mm, 3.7 mm, 4.0 mm was 0.025 seconds and the dwell time for the images recorded at 4.3 mm, 4.7 mm and 5.0 mm, was 0.033333 seconds, and the dwell time for the images recorded at 5.5 mm and 6.0 mm was 0.05 seconds, then the total review time from 0.0 mm to 0.6 mm would be 0.7 seconds, the same as if the redundant images had been culled.

In some embodiments, the tissue structure to be examined is the human torso. In other embodiments, the tissue structure to be examined is the human breast. In further embodiments, the tissue structure to be examined is the female human breast.

Some embodiments provide for a scanning completeness system for screening a defined volume of tissue having a manual image scanning device including an imaging probe, a system comprising three or more position sensors coupled with the image scanning device, a receiver to receive a set of discrete images from the image scanning device, a receiver to receive position data from locating system comprising three or more position sensors for each image in said set of discrete images, an image position tracking algorithm to determine the relative resolution of that set of discrete images of tissue within said defined volume, and a position tracking algorithm to determine the relative coverage of that set of discrete images of tissue, relative to another set of discrete images of tissue within that said defined volume. In further embodiments, the manual image scanning device is an ultrasound scanning device and the imaging probe is an ultrasound probe. In other embodiments, the manual image scanning device is an imaging device which utilizes ultrasound-derived properties including, but not restricted to, color Doppler and elastography.

In other embodiments, the position sensor can be a device which emits a magnetic or electromagnetic signal and locating system can include a device for sensing the relative position of the source of that magnetic or electromagnetic signal. In further embodiments, the position sensor can be a register which reflects electromagnetic radiation in the visible spectrum, or wavelengths between 750 nm and 390 nm, which may be detected by an optical camera and locating system can mean three or more optical cameras which can record the relative position between the register and the camera.

In another embodiment, the position sensor can be a register which reflects electromagnetic radiation in the infrared spectrum, or wavelengths between 100,000 nm and 750 nm, which may be detected by an infrared camera and locating system can include three or more infrared cameras which can record the relative position between the register and the camera. In a further embodiment, the position sensor can be a register which reflects electromagnetic radiation in the ultraviolet spectrum, or wavelengths between 390 nm and 10 nm, which may be detected by an ultraviolet camera and locating system can mean three or more ultraviolet cameras which can record the relative position between the register and the camera.

In some embodiments, the system comprises a storage device to store the discrete image data. In another embodiment, the system comprises a storage device to store the position sensor data corresponding to each discrete image. Further embodiments include a viewer to display the discrete images, wherein the viewer can provide a sequential display of said discrete images.

In some embodiments, the relative image resolution algorithm measures the three dimensional spacing between a pixel in one discrete image and a pixel at the same location of a second image recorded in a sequentially acquired image set. In other embodiments, an audible signal is issued in the event that the image resolution is not within a user-defined limit. In further embodiments, a visual signal is issued in the event that the image resolution is not within user-defined limits. In some embodiments, the visual signal identifies discrete image sequence wherein that the image resolution is not within user-defined limits.

In further embodiments, the image resolution algorithm creates a set of discrete image subsets by superimposing a three-dimensional volumetric boundary on adjacent images, determining which images have discrete image subsets which are described within that boundary, segregating the portions of each image subset which is described within that boundary, and calculating the pixels within the described subset of image portions.

In some embodiments, an image coverage algorithm measures the three-dimensional spatial distance the three dimensional locations of the edge boundaries of one set of sequentially-recorded images with a second set of sequentially-recorded images.

Other embodiments provide for a method for screening a defined volume of tissue with an image scanning device, comprising the following steps: scanning tissue within defined volume using a manual imaging probe; detecting the position of the imaging probe using three or more position sensors coupled with the imaging probe; receiving a set of discrete images from the image scanning device; receiving position data from locating system comprising three or more position sensors for each image in said set of discrete images; application of position tracking algorithm to determine the resolution of that set of discrete images of tissue within said defined volume; and application of position tracking algorithm to determine the relative coverage of that set of discrete images of tissue, relative to another set of discrete images of tissue within that said defined volume. In some embodiments, the manual image scanning device is an ultrasound scanning device and the imaging probe is an ultrasound probe. In some embodiments, a viewer is used to display discrete images, providing a, sequential display of said discrete images.

Some embodiments include one or more microprocessors to calculate the image resolution by calculating the three dimensional spacing between a pixel in one discrete image and a pixel at the same location of a second image recorded in a sequentially acquired image set.

Some embodiments provide for using one or more microprocessors to create a set of discrete image subsets by superimposing a three-dimensional volumetric boundary on adjacent images, determining which images have discrete image subsets which are described within that boundary, segregating the portions of each image subset which is described within that boundary, and calculating the pixels within the described subset of image portions.

In some embodiments, a locating system issues one or more audible signals in the event that the image resolution is not within user-defined limits to alert operator to obtain additional discrete images. In some embodiments, the locating system issues one or more visual signals in the event that the image resolution is not within user-defined limits to alert operator to obtain additional discrete images. In further embodiments, the visual signal identifies discrete image sequence wherein that the image resolution is not within user-defined limits to direct operator to location within defined volume requiring one or more additional discrete images.

In some embodiments, one or more microprocessors measure the three-dimensional spatial distance of the three dimensional locations of the edge boundaries of one set of sequentially-recorded images with a second set of sequentially-recorded images.

Some embodiments describe a method of displaying sequential images of tissue, wherein each image having assigned spatial coordinates, a discrete image display algorithm calculates the relative spacing between discrete images and modifies the rate of display of recorded discrete images to provide a uniform spatial-temporal display interval between successive discrete images. Other embodiments describe a method of displaying sequential images of tissue, wherein each image having assigned spatial coordinates, a discrete image display algorithm is used to determine whether a plurality of images are described within a user-defined interval for image spacing. Further embodiments provide that one or more of the plurality of images described within a user-defined interval for image spacing is not displayed as part of the set of discrete images.

Additional embodiments describe a method of displaying multiple sets of sequential images of tissue, wherein each image having assigned spatial coordinates, a discrete image display algorithm is used to not display one or more discrete images when the plane of that discrete images falls within a boundary of one or more sets of other sequential images.

Other objects of the invention will be obvious and will, in part, appear hereinafter. The invention, accordingly, comprises the method, system and apparatus possessing the construction, combination of elements, arrangement of parts and steps, which are exemplified in the following detailed description. For a fuller understanding of the nature and objects of the invention, reference should be made to the following detailed description taken in connection with the accompanying drawings.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 is a schematic view of the disclosed system including its various subsystem components.

FIG. 2 illustrates the hand-held ultrasound probe assembly including the affixed position sensors.

FIG. 3 illustrates an exploded view of the hand-held ultrasound probe assembly revealing the first and second support members, which encase the hand held ultrasound probe and incorporate the position sensors.

FIG. 4 illustrates a side view of the first support member shown in FIG. 3;

FIG. 5 illustrates a first transverse sectional view of the first support member shown in FIG. 3 revealing the conduits for incorporation of the position sensors and leads;

FIG. 6 illustrates a second transverse sectional view of the first support member shown in FIG. 3 revealing the conduits for incorporation of the position sensors and leads.

FIG. 7 illustrates a first cross-sectional view of the human breast including the hand-held ultrasound probe assembly shown at various positions during the course of a scan sequence.

FIG. 8A illustrates discrete images in a scan sequence.

FIG. 8B illustrates a second cross-sectional view of the human breast including the hand-held ultrasound probe assembly shown at various positions during the course of a scan sequence;

FIG. 9 illustrates a perspective view of the human breast and a ultrasound scan sequence including the hand-held ultrasound probe assembly shown at one position during the course of a scan sequence.

FIG. 10A illustrates a first top view of the human breast illustrating the locations of 14 scan sequences.

FIG. 10B illustrates a second top view of the human breast illustrating the locations of 13 scan sequences;

FIG. 10C illustrates a perspective view of the human breast illustrating the locations of 2 scan sequences and volume of tissue included within 2 scan sequences.

FIG. 10D illustrates a third top view of the human breast with a plurality of scan sequences.

FIG. 10E illustrates a fourth top view of the human breast with a plurality of scan sequences.

FIG. 10F illustrates two radial scan sequences.

FIGS. 10G-10L illustrate discrete images in two scan sequences.

FIG. 10M illustrates two radial scan sequences.

FIG. 11A-11F combine as labeled thereon to show a flow chart of the procedure associated with a described embodiment.

FIG. 12A illustrates the superposition of a single component volume unit on two sequential two-dimensional ultrasound scan images;

FIG. 12B illustrates the superposition of four component volume units at each of the corners of both planes of two sequential two-dimensional ultrasound scan images.

FIG. 13 is a schematic view of the disclosed system based on optical-based position sensing including its various subsystem components.

FIGS. 14A-14C illustrate a hand-held ultrasound probe assembly including affixed optically unique position sensors.

FIG. 15 illustrates an exploded view of a hand-held ultrasound probe assembly revealing the first and second support members, which encase the hand held ultrasound probe and incorporate the optically unique position sensors.

FIGS. 16A-16B illustrate the spacing between adjacent ultrasound scan images as a function of the depth of the ultrasound image within the tissue.

FIGS. 17A-17B illustrate a top view of a plurality of scan sequences with overlap.

### DETAILED DESCRIPTION

As described briefly above, embodiments contemplated provide for methods, devices, systems that can be used with manual imaging techniques to ensure satisfactory quality and adequate completeness of a scanning procedure for a patient's target region. Some embodiments employ rapid-response position sensors or rapidly imaged optical registers affixed to an existing hand-held imaging system, for example, a diagnostic ultrasound system, and associated hand-held imaging probes. By way of example, one type of ultrasound system that can be used with some embodiments described is the Phillips iU22 xMatrix Ultrasound System with hand-held L12-50 mm Broadband Linear Array Transducer (Andover, Mass.). Also, a commercially available system which provides accurate x, y, z position coordinates for multiple sensors as a function of time, providing said position information at a rapid tracking rate, is, by way of example, the Ascension Technology 3D Guidance trakSTAR (Burlington, Vt.).

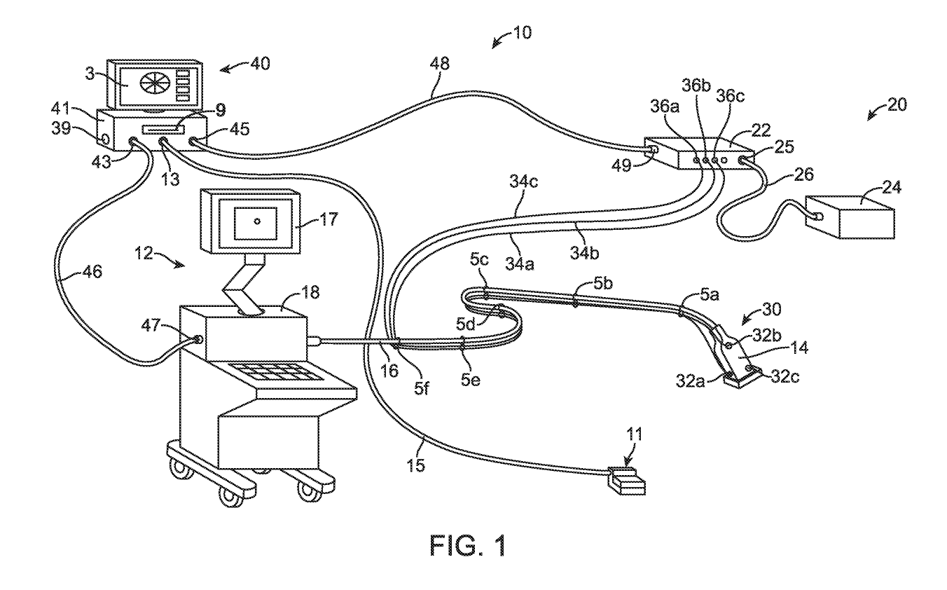

Referring to FIG. 1, two principal subsystems are illustrated. A first subsystem is the hand-held imaging system 12, which includes hand-held imaging monitor console 18, display 17, hand-held imaging probe 14 and connecting cable 16. A second system (referred to hereinafter as the “Scan Completeness Auditing System”), according to the invention, is represented in general at 10. The Scan Completeness Auditing System 10 comprises a data acquisition and display module/controller 40 including microcomputer/storage/DVD ROM recording unit 41, display 3 and footpedal or other control 11. Foot pedal 11 is connected to microcomputer/storage/DVD ROM recording unit 41 via cable 15 and removably attachable connector 13. The Scan Completeness Auditing System 10 also comprises position-tracking system 20, which includes, by way of example, position tracking module 22 and position sensor locator, such as a magnetic field transmitter 24. In addition, the Scan Completeness Auditing System 10 also comprises a plurality of position sensors 32a, 32b and 32c affixed to the hand-held imaging probe 14. Although the hand-held imaging system 12 is shown as a subsystem separate from the scanning completeness auditing system 10, in some embodiments, the two systems are part of the same overall system. In some cases, the imaging device may be part of the scanning completeness auditing system.

Still referring to FIG. 1, hand-held imaging system 12 is connected to data acquisition and display module/controller 40 via data transmission cable 46 to enable each frame of imaging data (typically containing about 10 million pixels per frame) to be received by the microcomputer/storage/DVD ROM recording unit 41 the frequency of which is a function of the recording capabilities of the microcomputer/storage/DVD ROM recording unit 41 and the image data transmission capabilities, whether it is raw image data or video output of the processed image data, of the hand-held imaging system 12. Position information from the plurality of position sensors 32a, 32b, and 32c, is transmitted to the data acquisition and display module/controller 40 via the transmission cable 48. Cable 46 is removably attached to microcomputer/storage/DVD ROM recording unit 41 of data acquisition and display module/controller 40 with removably attachable connector 43 and is removably connected to diagnostic ultrasound system 12 with connector 47. The successive scans associated with the hand-held imaging procedure are stored and subjected to computational algorithms to assess completeness of the diagnostic ultrasound scanning procedure as described in greater detail in the specifications which follow.

Still referring to FIG. 1, position tracking module 22 is connected to data acquisition and display module/controller 40 via data transmission cable 48 wherein cable 48 is removably attached to microcomputer/storage/DVD ROM recording unit 41 of data acquisition and display module/control 40 with connector 45 and is removably connected to position tracking module with connector 49. Position sensor locator, such as a magnetic field transmitter 24 is connected to position tracking module 22 via cable 26 with removably attachable connector 25. Hand-held imaging probe assembly 30 seen in FIG. 1 includes, by way of example, position sensors 32a-32c, which are affixed to hand-held imaging probe 14 and communicate position data to position tracking module 22 via leads 34a-34c, respectively, and removably attachable connectors 36a-36c, respectively. Position sensor cables 34a-34c may be removably attached to ultrasound system cable 16 using cable support clamps 5a-5f at multiple locations as seen in FIG. 1

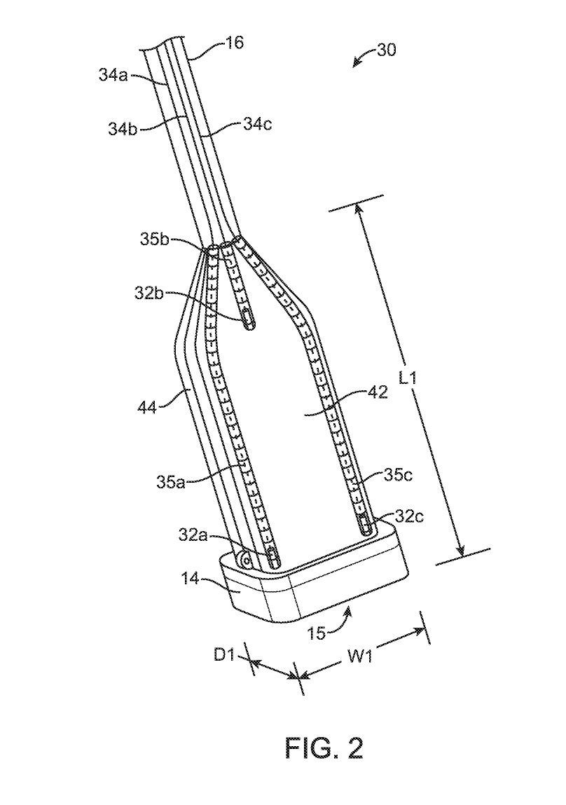

Referring now to FIG. 2, the position-sensor instrumented hand-held imaging probe is described in greater detail. In one embodiment, the hand-held probe assembly 30, a hand-held imaging probe 14 is enclosed within first and second “clamshell” type support members 42 and 44, respectively. First support member 42 incorporates three raised ridges 35a-35c, which provide three conduits (not shown) for position sensors 32a-32c, respectively, and position sensor cables 34a-34c, respectively.

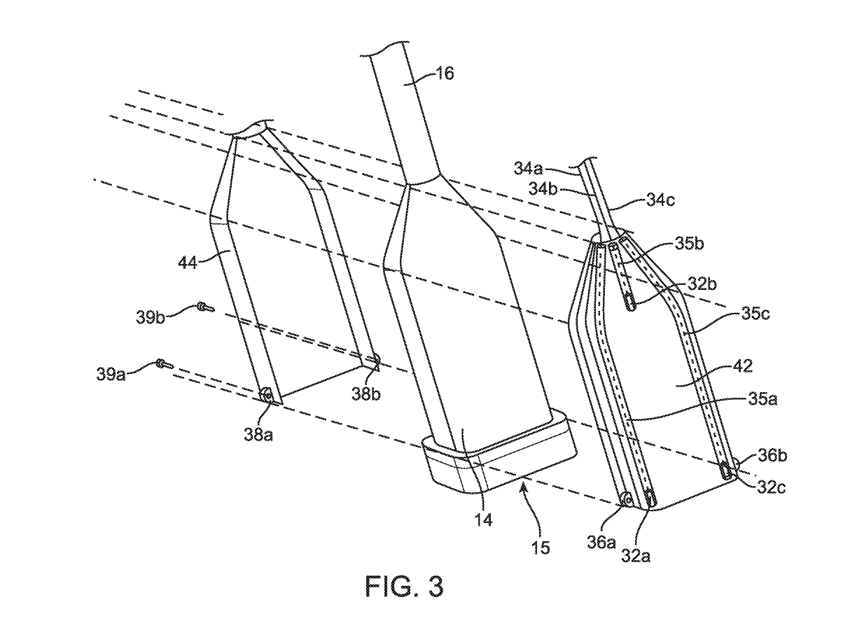

Another embodiment is further illustrated in an exploded view of the hand-held probe assembly 30 as seen in FIG. 3. Said first support member 42 includes the aforementioned raised ridges 35a-35c and associated conduits 33a-33c, respectively, which accommodate position sensors 32a-32c and their corresponding cables 34a-34c, respectively. First support member 42 also incorporates extension ears 36a and 36b, each with a drilled hole to enable secure mechanical attachment to second support member 44. Said second support member 44 likewise incorporates extension ears 38a and 38b, each with a drilled hole which matches drilled holes in first support member to enable secure mechanical attachment to second support member 42 using screws 39a and 39b, respectively. First and second support members may be manufactured using a non-ferromagnetic metal or alloy or, preferably, an injection molded plastic. The interior contours and dimensions of the first and second support members 42 and 44 are designed to match the particular contour and dimensions of the off-the-shelf hand-held ultrasound probe being instrumented with the position sensors 32a-32c. Accordingly, the contours and dimensions of the first and second support members 42 and 44 will vary according the hand-held ultrasound probe design. The exact location of the position sensors 32a-32c relative to the ultrasound transducer array at the end face of the hand-held imaging probe (not shown) will accordingly be known for each set of first and second support members since they are designed to attached to and operate in conjunction with a specific hand-held ultrasound probe.

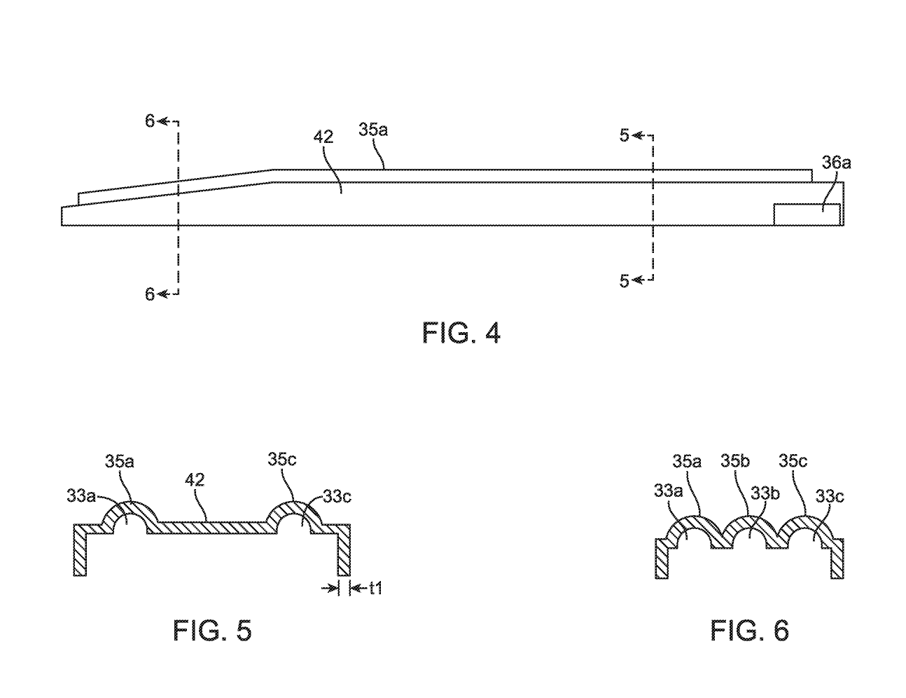

Additional features of first support member 42 are revealed in FIGS. 4, 5 and 6 which illustrate an embodiment of the first support member 42 in a side view (see FIG. 4) and sectional views (see FIGS. 5 and 6) at two locations along the length of first support member 42. As seen in FIG. 4, the raised ridge 35a is seen which extends along most of the length of first support member 42. Also, extension ear 36a is seen one end of the first support member 42. Referring to FIGS. 5 and 6, which provides transverse cross-sectional views of first support member 42, conduits 33a, 33b and 33c are revealed. The dimensions of conduits 33a-33c are selected to accommodate position sensors 32a-32c and their corresponding cables 34a-34c, respectively. By way of example, position sensors are commercially available which have a diameter of nominally 2 mm or less. Accordingly, one described embodiment provides conduits 33a-33c dimensioned to accommodate a 2 mm diameter position sensor. As seen in FIGS. 2, 3, 5 and 6, position sensors 32a-32c and their respective cables 34a-34c can be affixed within conduits 33a-33c using an adhesive (e.g., epoxy or cyanoacrylate).

Returning to FIG. 2, by way of example, the typical dimensions of a hand-held ultrasound probe 14 are provided below:
W1=1.5 to 2.5 inches
L1=3 to 5 inches
D1=0.5 to 1 inch

Accordingly, as specified in the previous paragraph, the first and second support members 42 and 44 are sized to correspond to the particular contour and dimensions of a specific hand-held ultrasonic probe design. For the case of injection-molded plastic, e.g., a biocompatible grade of polycarbonate, the inner dimensions of said first and second support members 42 and 44 are designed to closely match the outer dimensions of the hand-held ultrasound probe 14. The wall thickness, t1 (see FIG. 5) of the injection molded plastic support members 42 and 44 is preferably in the range from 0.05 to 0.10 inch.

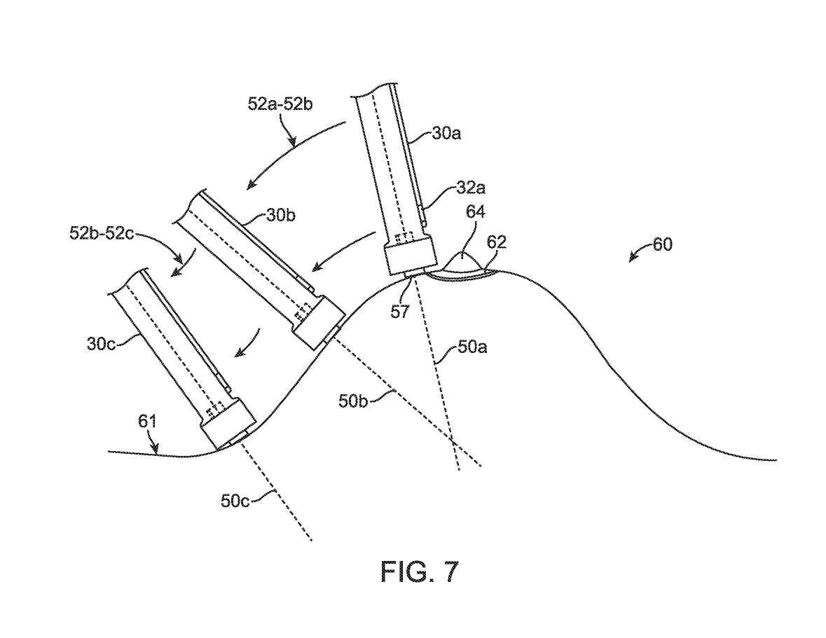

An example of the use of described embodiments is seen in FIG. 7 for the case of the hand-held ultrasound examination of a human breast 60. In the example seen in FIG. 7, a hand-held ultrasound probe assembly 30 with affixed position sensors is illustrated at a starting position on the human breast 60 adjacent to the nipple 64 and areola 62. In an example hand-held ultrasound scanning procedure of the human breast 60, the hand-held ultrasound probe assembly 30 starts immediately over the nipple and progresses radially and follows the contour of the human breast as illustrated by translation vectors 52a-52b and 52b-52c corresponding to hand-held ultrasound probe assembly 30 successive positions 30a, 30b and 30c with the latter two positions shown in “phantom” format. During the scan sequence, the ultrasound transducer array 57 is maintained in direct contact with the skin, usually with an intervening layer of an ultrasound coupling gel. An ultrasound coupling gel is usually used (e.g., Aquasonics 100, Parker Laboratories, Inc., Fairfield, N.J.) to improve ultrasound interrogation by providing an improved acoustic pathway between the ultrasound transducer array and the skin.

By way of example, the hand-held ultrasound probe assembly 30 is moved by the operator using a manual technique along the pathway illustrated in FIG. 7, referred to herein as a single scan sequence, beginning at the nipple 64 and ending when the ultrasound transducer array has reached the surface of the chest 61 beyond the perimeter of the breast 60, or beginning at the chest wall and ending when the ultrasound transducer has reached the nipple. If this example scan sequence is performed within the acceptable limits of translation speed and rate of change of the orientation of the hand-held ultrasound probe assembly 30, then this scan sequence would be verified as a complete scan sequence. As seen in FIG. 7, a planar ultrasound beam 50a-50c is emitted and a corresponding ultrasound image is obtained at each momentary position 30a-30c of the hand-held ultrasound probe assembly 30. As the hand-held ultrasound probe assembly 30 is translated along the illustrated scan sequence path in FIG. 7, an ultrasound beam is emitted and an image is received, constituting a single image frame, at a rate in the range from about 10 to 40 times (or frames) per second. A typical frame may contain an array of 400×600 pixels of image data or 240,000 pixels per frame. A new frame is obtained at a rate of about 10 to 40 frames per second.

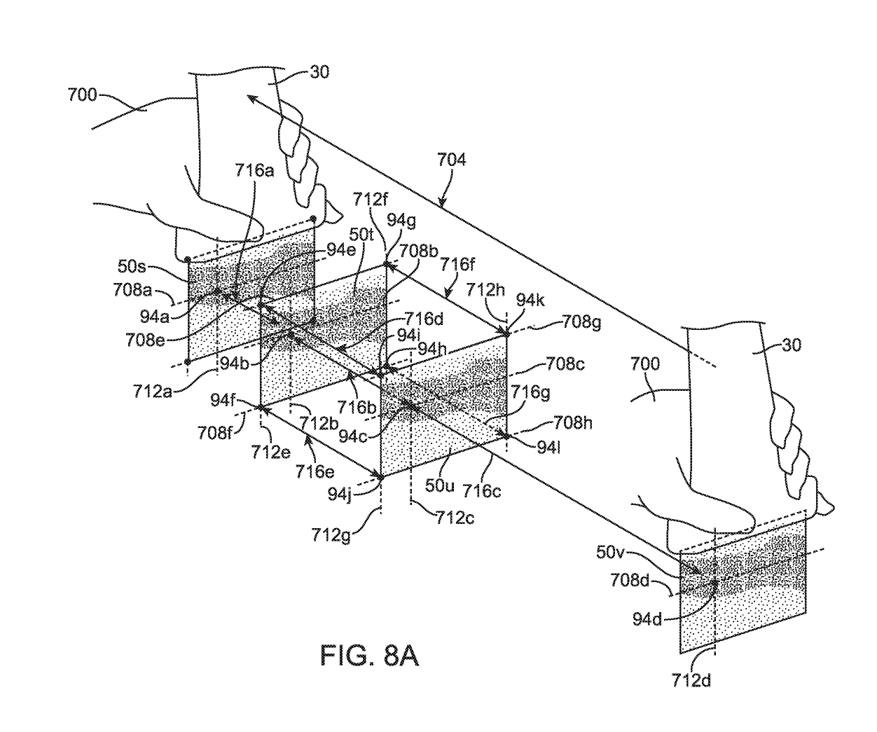

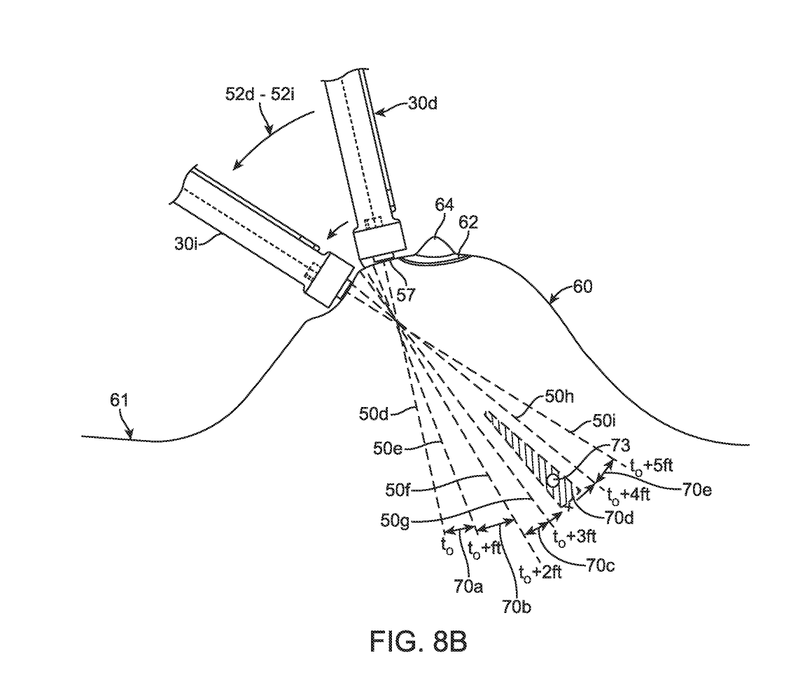

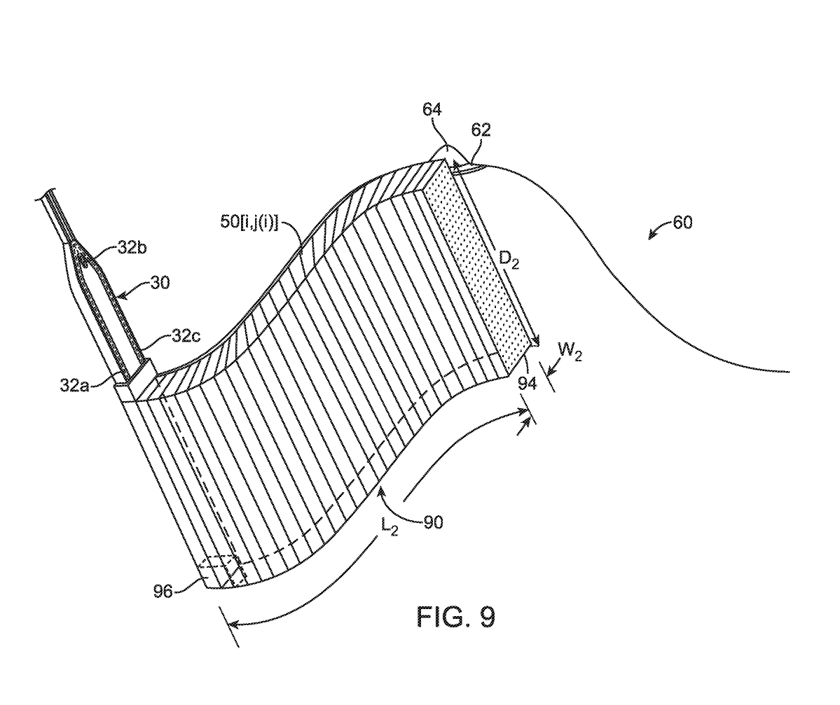

An important aspect of the present invention is illustrated in FIGS. 8A, 8B, and 9 related to computing (or auditing) the completeness of each scan sequence. This described method and algorithm assures the frame-to-frame resolution of any individual scan sequence (e.g., any individual path scanned beginning at the nipple of the breast and ending at the chest surface beyond the perimeter of the breast boundary, or scan beginning at the chest surface and ending at the nipple, or any scan beginning at the clavicle and ending at the base of the rib cage, or any scan beginning at the base of the rib cage and ending at the clavicle, or any scan beginning in the crevice of the armpit and ending at the inferior lateral side of the rib cage).

In some embodiments, measuring or calculating the spacing or distance between individual images in a scan sequence may be referred to as determining the image-to-image resolution or spacing between discrete images in a scan sequence. Alternatively, frame to frame resolution may also be used to describe the spacing/distance between images in a scan sequence.

By way of example and referring first to FIG. 8.A, the hand-held ultrasound probe assembly 30 is translated across the surface of the skin by the human hand 700. That translation will follow a linear or non-linear path 704, and there are a series of corresponding ultrasound beam positions 50s-50v, each with a corresponding ultrasound image that is recorded, as depicted in FIG. 1, by the acquisition and display module/controller 40 via the data transmission cable 46, to be received by the microcomputer/storage/DVD ROM recording unit 41, the frequency of which is a function of the recording capabilities of the microcomputer/storage/DVD ROM recording unit 41 and the image data transmission capabilities. Again referring to FIG. 8A, the images are stored as a set of pixels, including pixels 94a-94l, which are displayed in a two-dimension matrix of pixels, each matrix consisting of horizontal rows 708a-708h and vertical columns 712a-712h. A single pixel 94a-94h, is displayed has a unique display address P(rₓ, cₓ), where rₓ is the row of pixels on the image, r₁ being the row at the top, e.g. 708e, or the row representing structures closest to the probe, and rₗₐₛₜ being the row at the bottom (e.g. 708f), or the row representing structures furthest away from the probe; and where cₓ is the column of pixels on the image, c₁ being the column on the left (as viewed by the reviewer, e.g. 712g), and cₗₐₛₜ being the column on the right (as viewed by the reviewer, e.g. 712h). A typical recorded ultrasound image will have between 300 and 600 horizontal rows 708 and between 400 and 800 vertical columns 712. Thus, a typical recorded ultrasound image shall have between 120,000 and 480,000 pixels 94.

Referring again to FIG. 8A, the recorded image for each ultrasound beam position 50s-50v will have an identical pixel format. A corresponding row is the row 708 which is displayed at the same distance, vertical from the top, in every image. The depth, as measured as distance away from probe, shall be the same for corresponding horizontal rows 708. In the way of example, the information in the 8^{th} horizontal row 708 in one image represents structures which are the same distance, away from the probe at the time they are recorded, as the location of the information in the 8^{th} horizontal row 708 in another image at the time that image is recorded. The same logic applies to the corresponding vertical columns 712. By way of example, the information in the 12^{th} vertical column 712 in one image represents structures that are the same distance, horizontally, from the center of the probe at the time that image is recorded as the location of the information in the 12^{th} vertical column 712 in another image at the time it is recorded. Thus, the information described any one pixel 94, P(rₓ, cₓ), in one image is the same distance away from the surface of the probe (depth) and from the center line of the probe as the information described at the same pixel 94 location P(rₓ, cₓ), in another image. These pixels 94 that share common locations on the image format for the discrete images in the image sets are termed corresponding pixels 94.

One embodiment for calculating the completeness of the scan sequence in terms of frame-to-frame resolution is to calculate the maximum distance between any two adjacent image frames. Since the concept of minimum acceptable resolution, by definition, requires the establishment of a maximum acceptable spacing, then that resolution requirement will be met if the largest distance 716 between any two corresponding pixels 94 in adjacent image frames is within the acceptable limit. Since the frames are planar, then the largest distance between any two frames will occur at the corresponding pixels 94 that are at one of the four corners. Thus, the maximum distance 716 between any two corresponding frames shall be (EQ. 1):

{Maximum Distance between any Two Corresponding Frames}==MAX(DISTANCE(P(FIRST-ROW, FIRST-COLUMN)−P′(FIRST-ROW, FIRST-COLUMN)), DISTANCE(P(FIRST-ROW, LAST-COLUMN)−P′(FIRST-ROW, LAST-COLUMN)), DISTANCE(P(LAST-ROW, FIRST-COLUMN)−P′(LAST-ROW, FIRST-COLUMN)), DISTANCE(P(LAST-ROW, LAST-COLUMN)−P′(LAST-ROW, LAST-COLUMN)))
Where P and P′ are the corresponding pixels 94 in two adjacent images, MAX is the maximum function which chooses the largest of the numbers in the set (in this example 4) and DISTANCE is the absolute distance 716 between the corresponding pixels.

Exemplary distances are shown in FIG. 8A at 716a between pixel 94a and corresponding pixel 94b; 716b between pixels 94b and 94c; 716c between 94c and 94d; 716d between 94e and 94i; 716e between 94f and 94i; 716f between 94g and 94k; and 716g between 94i and 94l. This method of assuring frame-to-frame resolution may be used to assure that the resolution remains within limits regardless of the speed of longitudinal translation of the probe, speed of lateral rotation of the probe, speed of axial resolution of the probe, or speed of vertical rotation of the probe. If the distance between pixels exceeds an acceptable spacing/distance then the user may be prompted during or at the end of the process/procedure to rescan a region. In some cases, the acceptable spacing/distance is a preselected or predetermined value. In some cases, the value is a user defined limit. In other embodiments, the system may provide a range or acceptable spacing/distances for selection based on the type of exam or characteristics of the patient or target region for scanning.

FIG. 8B provides another method of assuring adequate frame-to-frame or image-to-image spacing. FIG. 8B shows the hand-held ultrasound probe assembly 30 at two adjacent positions 30d and 30i. For this example, assume that the rate of producing new ultrasound images is accomplished at a rate of 10 frames/second. As the hand-held ultrasound probe assembly 30 is translated from position 30d with corresponding ultrasound beam 50d and a corresponding ultrasound image to position 30i with corresponding ultrasound beam position 50i and a corresponding ultrasound image, there are 4 intermediate positions as seen by ultrasound beams 50e-50h. Also, assume that the rate of longitudinal rotation of the hand-held ultrasound probe assembly 30 during the translation from position 30d to 30i is not uniform and an increased rate of rotation of the hand-held ultrasound probe assembly 30 inadvertently occurs between ultrasound beam 50g and 50h. For the case of the example illustrated in FIG. 8B, the time step, δt is 0.10 second based on an ultrasound scan rate of 10 frames per second. As a result of a faster than allowed rate of rotation between beam position 50g and 50h and corresponding ultrasound images, a set of omitted zones 70a-70e within the targeted tissue (i.e., the human breast 60 in this example) are not included in the ultrasound scan sequence. As a consequence, if a suspicious lesion 73 were within omitted zone 70d, it would not be detected or recorded in the diagnostic ultrasound procedure. Unavoidably, it would be impossible for the expert (e.g., radiologist) who analyzes the ultrasound images following the ultrasound procedure to detect the presence of what could become a life-threatening malignant lesion. It is not mathematically possible to eliminate these omitted zones 70a-70e without an infinite number of ultrasound beams 50d-50i and corresponding ultrasound images, but the user can determine a level of resolution, that is the maximum acceptable size, of the zones 70a-70e and notify the user if any one of those zones exceeds that acceptable limit.

Still referring to FIG. 8B, a preferred algorithm for computing spacing between images in a scan (e.g. image-to-image spacing) is to compute the maximum chord or distance, x between successive planar ultrasound scan frames at the maximum intended depth of ultrasound interrogation (i.e., maximum depth of the breast tissue in the present example). This maximum distance, x can be computed between the distal boundaries of each successive ultrasound scan frame (e.g., between ultrasound beam 50g and 50h, and corresponding images, since the position of the ultrasound transducer array 57 and the orientation of the hand-held ultrasound probe assembly 30 is precisely known at all time points when ultrasound scan frames are generated and recorded. For the case of one embodiment of the present invention involving the use of the Ascension Technologies position sensor product, the position of each sensor is determined (in one example version of a product sold by Ascension Technologies but not intended as a limitation as the data update rate may be higher or lower) at a rate of 120 times per second which is an order of magnitude more frequently than the repetition rate for ultrasound scan frames. As a consequence, the precise location of the ultrasound scan frame and, thereby, the precise location of the 240,000 pixels within each ultrasound scan frame, will be known in three-dimensional space as each ultrasound scan frame is generated by the ultrasound system 12 and recorded by the data acquisition and display module/controller 40. According, knowing the position of all pixels within each successive frame will enable the maximum distances between corresponding pixels in successive frames to be computed, focusing on those portions of successive ultrasound beams 50d-50h, and corresponding ultrasound images, that are known to be furthest apart, i.e., at locations within the recorded scan frame most distant from the ultrasound transducer array 57.

Referring now to FIG. 9, another algorithm for computing the acceptability of the speed of translation and/or the rate of change of the orientation of the hand-held ultrasound probe assembly 30 is illustrated. This alternative method and algorithm for assuring the completeness of any individual scan sequence (e.g., any individual path scanned beginning a the nipple of the breast and ending at the chest surface beyond the perimeter of the breast boundary) involves computation of the pixel density in each unit volume 96 within the swept volume 90 of the scan sequence, i containing N ultrasound beams 50[i,j(i)] and associated recorded frames where i equals the number of scan sequences and j(i) equals the number of emitted beams 50 and associated recorded frames for each scan sequence, i. By way of example and still referring to FIG. 9, assume that the rate of translation of the hand-held ultrasound probe assembly 30 along scan sequence, i, having path length, L2, is 1.0 cm/second, length L2 equals 15 cm and the ultrasound system 12 scanning rate is 10 frames/second and the resultant images are recorded by the data acquisition and display module/controller 40 at 10 frames/second. Based on these example parameters, the total time to complete the scan is 15 seconds and the total number of ultrasound scan frames recorded is 150. In this example, j(i) equals 150. If each frame contains, for example, 240,000 pixels, then the total volume will include 150 frames×240,000 pixels/frame which equals a total of 36 million pixels in the swept volume 90 of an individual scan sequence, i. Since the precise position and computed orientation of the hand-held ultrasound probe assembly 30, its ultrasound beam 50[i,j(i)] and its associated frame of pixels are known at the moment of each recorded frame, then the precise location of the plane in which each pixel 94 resides within the swept volume 90 can be computed.

Still referring to FIG. 9, according to the teachings of this invention, the swept volume 90 of the scan sequence would be the volume defined by (a) the width, W2 of the ultrasound beam, which is defined by the length of the ultrasound transducer array (e.g., 5 cm), (b) the depth, D2 of the recorded penetration of the ultrasound beam into the targeted living tissue (e.g., 5 cm) and (c) the total length, L2 traversed in an individual scan sequence (e.g., 15 cm). This total volume (375 cubic cm in the present example) is then subdivided into unit volumes exemplified by unit volume 96 (e.g., cubical volume of dimensions 1.0 cm×1.0 cm×1.0 cm). For this example, the swept volume 90 would be subdivided in to 375 unit volumes 96. The number of ultrasound scan pixels 94 contained in each unit volume 96 is computed and this number is compared to a predetermined Minimum Pixel Density number. By way of example, but not limiting the invention, the number of ultrasound scan pixels 94 within a unit volume 96 may be computed by comparing the x-y-z coordinates of each of the ultrasound scan pixels 94 in the 150 frames which comprise the swept volume 90, with the x-y-z coordinates of the boundaries of the perimeter of the unit volume 96. If the x-y-z coordinates of the ultrasound scan pixel 94 is within the boundaries of the perimeter of the unit volume 96, it is counted. If the x-y-z coordinates of the ultrasound scan pixel 94 is outside of the boundaries of the perimeter of the unit volume, it is not counted. If the computed pixel density within any unit volume 96 (i.e., any of the 375 unit volumes in this example) within the swept volume 90 is less than the Minimum Pixel Density, then the operator is alerted at the end of the scan sequence that scan sequence just completed is incomplete and that all or part of it must be repeated, or that the operator must accept that the scan sequence is incomplete. Said alert includes a display of the scan path just completed as well as instructions to the operator to improve scanning method to achieve a complete scan. For example, these instructions include reducing the scanning speed and/or the rate of change of orientation of hand-held ultrasound probe during the repeated scan sequence.

In some embodiments, the range of the image-to-image resolution (spacing) within each scan sequence is a pixel density between 9,000 and 180,000,000 pixels/cm³. In other embodiments, the pixel density is between 22,500 and 18,000,000 pixels/cm³. In further embodiments, the pixel density is between 45,000 and 3,550,000 pixels/cm³.

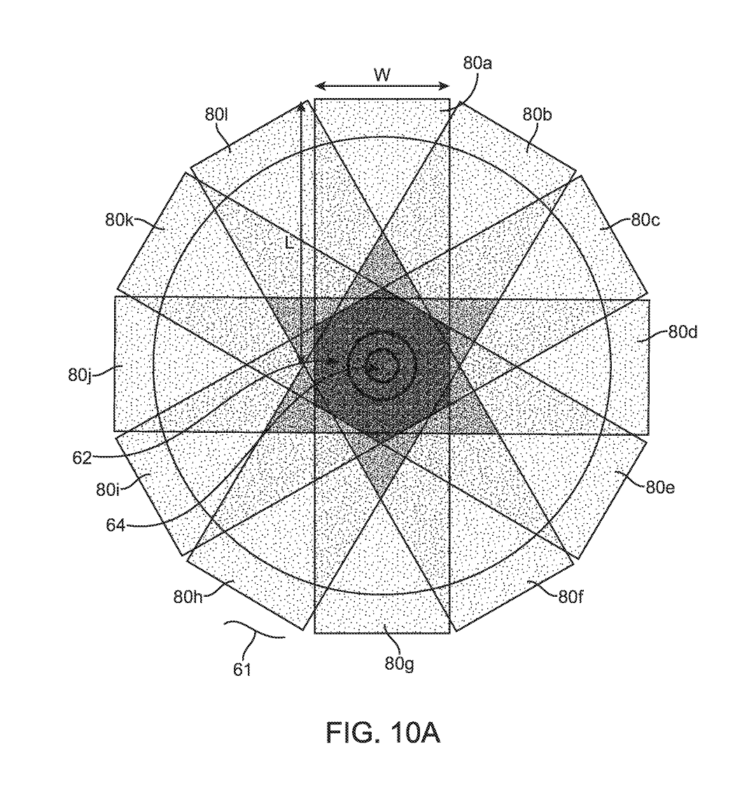

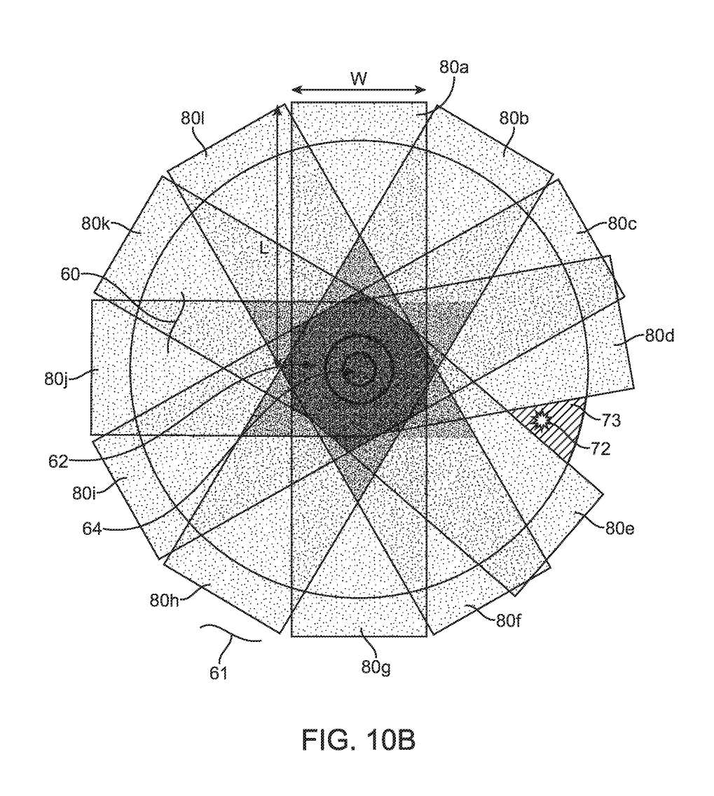

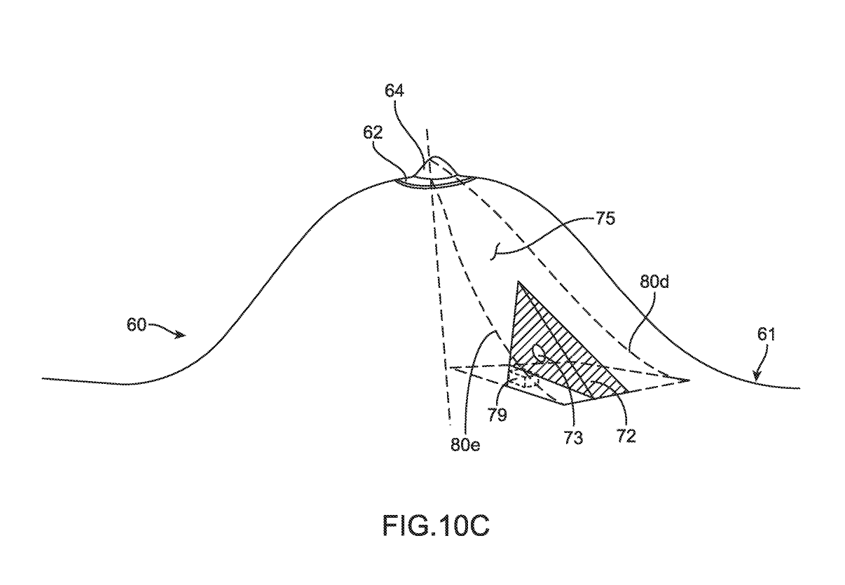

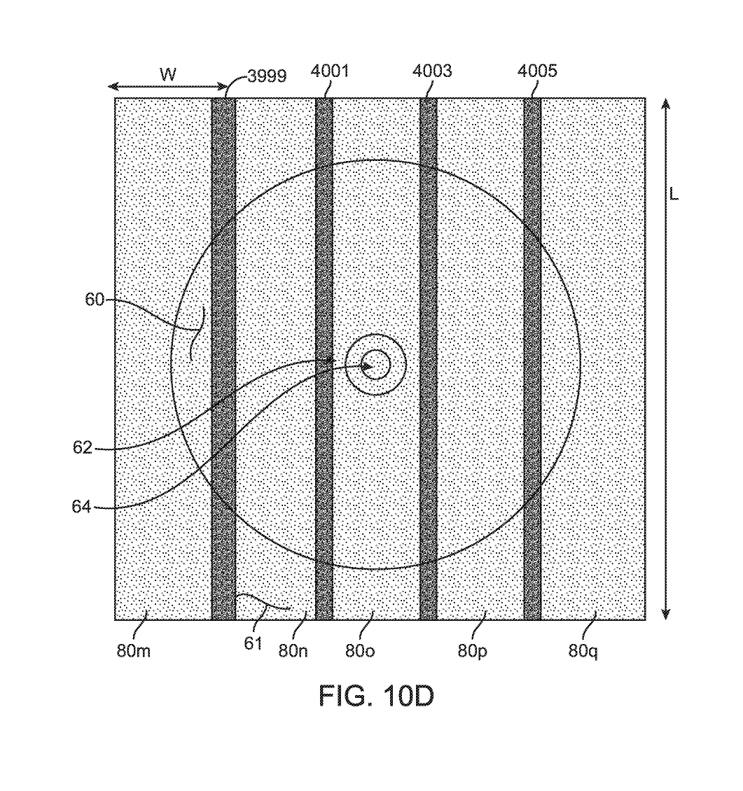

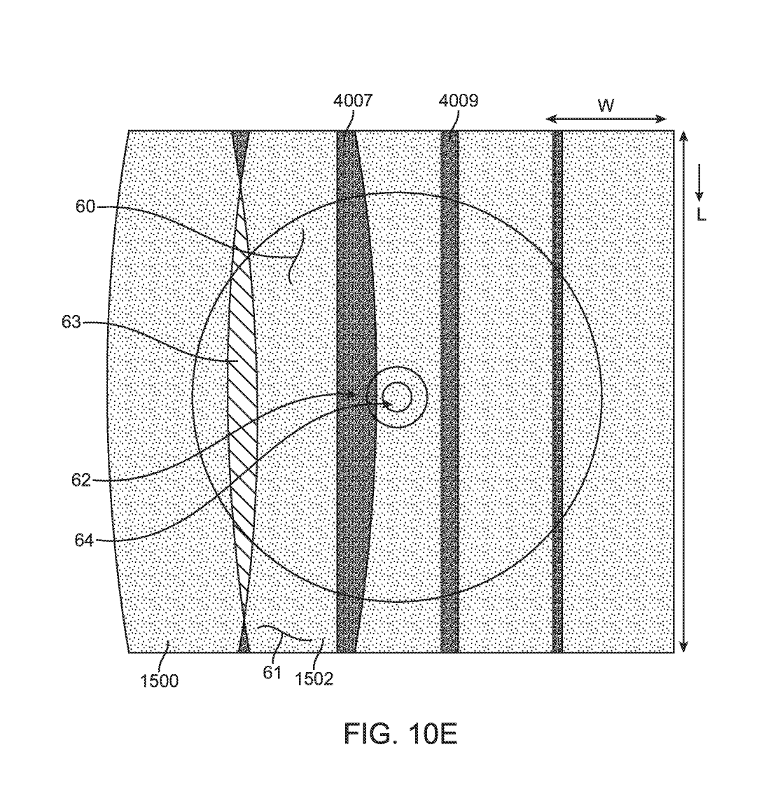

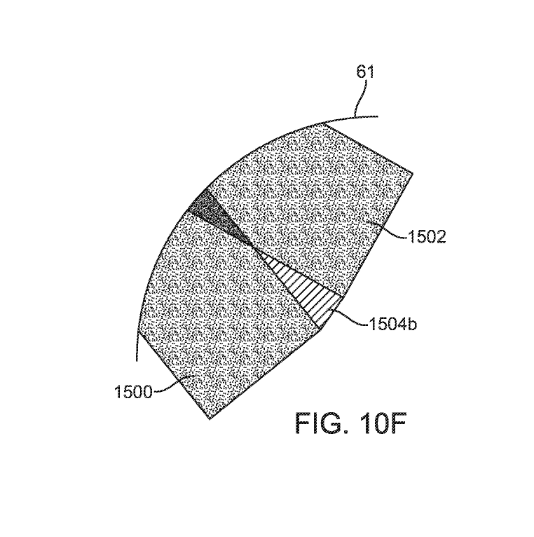

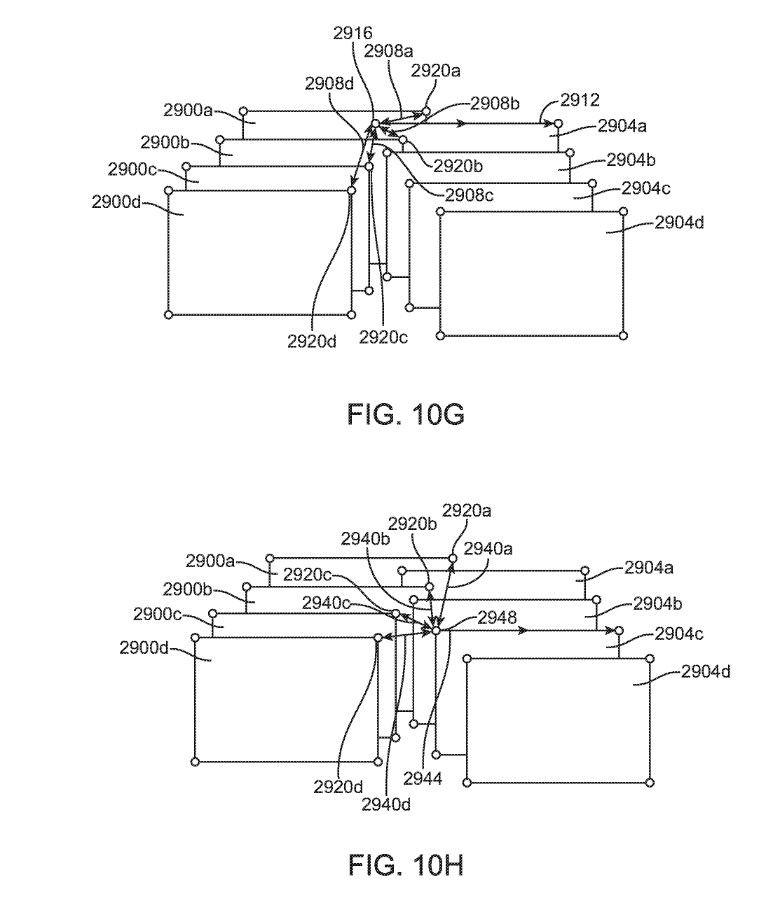

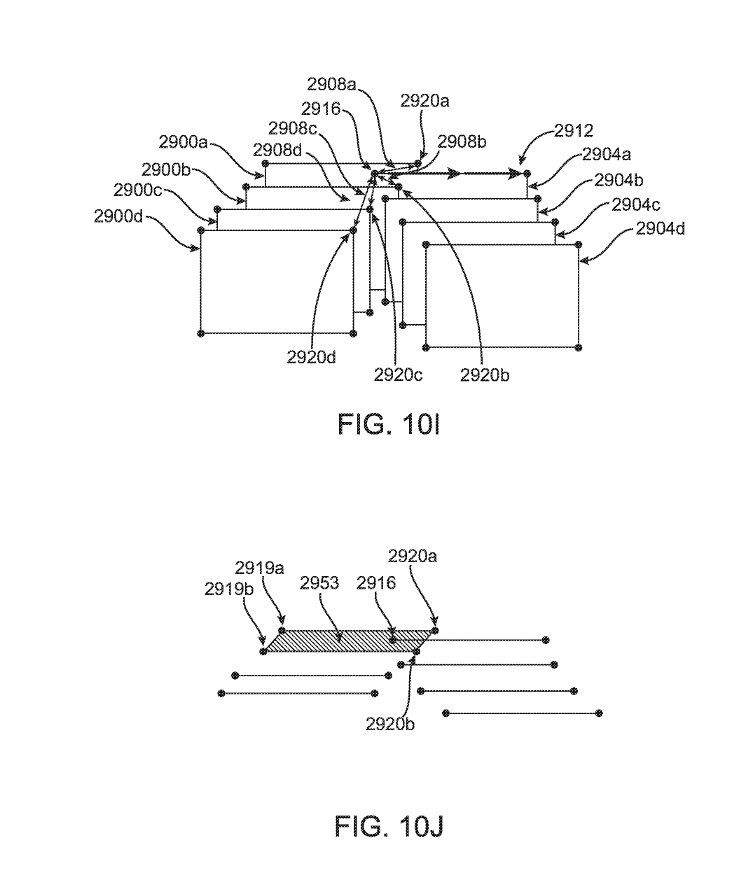

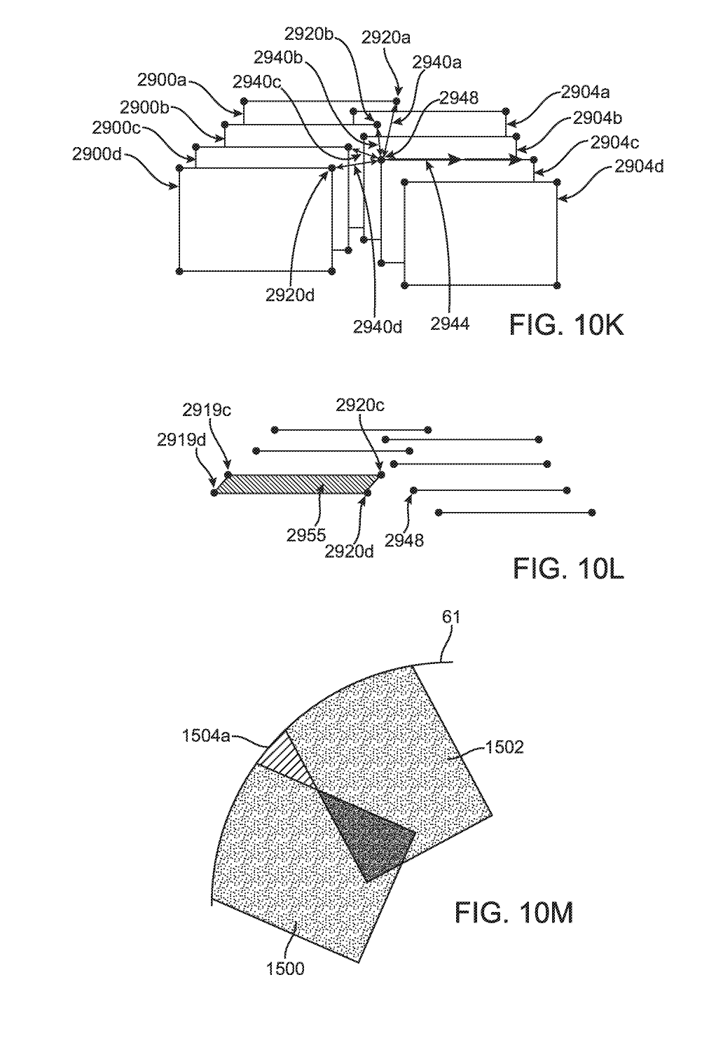

An equally important aspect of the present invention is illustrated in FIGS. 10A and 10B related to computing (or auditing) the tissue coverage by comparing the scan sequence just completed based on its relative distance from the previously completed scan sequence. According to the teachings of this invention and referring to FIG. 10A, the accurate and dynamic computation of the position of the hand-held ultrasound probe's transducer array enables the computation of the actual spatial position and computed orientation of sequential and manually scanned pathways completed along the tissue surface. By way of example, relatively uniformly and closely spaced radial scan sequences 80a-80l are superimposed on a top view of the human breast 60 as seen in FIG. 10A with scan sequences 80 spanning the distance between the nipple 64 and some distance radially outward from the nipple, for example, the chest surface 61. Each scan sequence 80 has a length L and a width W. The computed position and computed orientation of each sequential and manually derived scan sequence 80a-80l scanned along the tissue surface enables the further computation of the physical spacing between the boundaries of each adjacent and successive scan sequence 80. This computation can be rapidly completed during the course of the manual scanning process and a visual and audible cue as well as an image is provided showing the paths of completed scan sequences to identify where re-scanning is required. This intra-procedure computation of the distances between adjacent scan sequences, 80a-80l assures that complete coverage of the ultrasound scan of the targeted tissue region is achieved by identifying any completed scan sequences that are separated by an unacceptably large distance.

Referring now to FIG. 10B, radial scan sequences 80a-80l are superimposed on a top view of the human breast 60 with scan sequences 80 spanning the distance between the nipple 64 and the chest surface 61. In contrast to the example seen in FIG. 10A, this example illustrates an abnormally large spacing between scan sequence 80d and 80e. As a consequence of an inadvertently large spacing between scan sequences 80d and 80e, a zone 72 (as revealed by shaded region in FIG. 10B) by of tissue within the breast 60 is not included in the diagnostic ultrasound procedure. The distance between successive scan sequences can be computed since the precise location and computed orientation of the hand-held ultrasound probe assembly 30 is known for each scan sequence 80. If the spacing between scan sequences exceeds a predetermined maximum distance between successive scans, then a visual and audible cue is issued as well as an image is displayed showing the paths of completed scan sequences to identify where re-scanning is required. This intra-procedure computation of the distances between adjacent scan sequences assures that a complete diagnostic ultrasound scan of the targeted tissue region is achieved by identifying any completed scan sequences that are separated by an unacceptably large distance.

Still referring to FIG. 10B, the result of a computed physical spacing between successive scan sequences 80d and 80e being greater than a predetermined maximum spacing value is an un-scanned or omitted zone 72 within the targeted tissue (i.e., the human breast 60 in this example). As a consequence, if a suspicious lesion 73 were within omitted zone 72, it would not be detected or recorded in the diagnostic ultrasound procedure. Unavoidably, it would be impossible for the expert (e.g., radiologist) who subsequently analyzes the recorded ultrasound images following the diagnostic ultrasound procedure to detect the presence of what could become a life-threatening malignant lesion.

Similarly, FIGS. 10D and 10E show scan-to-scan spacing between relatively linear scan sequences. FIG. 10D shows scan sequences 80m-80q following a substantially linear pathway across the breast 60. The sequences show overlapping imaging at 3999, 4001, 4003, and 4005. FIG. 10E, on the other hand, illustrates a gap of unscanned tissue between scan sequence 1500 and scan sequence 1502. In such circumstances, embodiments described would be used to calculate, measure, or determine the size of the unscanned region 63. If the distance is greater than an acceptable spacing for scan-to-scan spacing, then the operator would be alerted during the procedure to scan the region 63.

FIGS. 10F and 10M show scan-to-scan spacing between relatively radial scan sequences. Two scan sequences 1500 and 1502 show unscanned regions 1504a and 1504b. In such cases, embodiments described would be used to calculate, measure, or determine the size of the unscanned region. If the distance is greater than an acceptable spacing for scan-to-scan spacing, then the operator would be alerted during the procedure to scan the region.

In some embodiments, measuring or calculating the spacing or distance between scan sequences may be referred to as determining the scan-to-scan spacing between scan sequences. Scan-to-scan spacing is a method of measuring, calculating, or otherwise determining coverage. If the images in the scan sequences overlap, there is coverage. If there is a gap between the two scan sequences, there is incomplete coverage.

Referring to FIG. 10G, two adjacent scan sequences 2900a-2900d and 2904a-2904d are depicted. One means of measuring whether there is overlap or gap spacing is to measure the distances 2908a-2908d from one of the corner pixels of one image, for example P(FIRST-ROW, LAST-COLUMN) 2916 and each of the pixels in the same row, but opposite side of the image in all of the images in the adjacent row, for example P(FIRST-ROW, FIRST-COLUMN) 2920a-2920d. The shortest of those distances represents the spacing between adjacent images in adjacent rows. In the example of FIG. 10G, that would be distance 2908b. If the vector of that distance, that is the vector from 2916 to 2920b, shown at 2913, is in the same general direction as the vector which emanates from that corner pixel and the pixel on the same row, but opposite side of the image 2912, as is the case of the vector between 2916 and 2920b (2913) and the vector 2912, then the distance between the corner pixels of the two adjacent images represents an overlap. In other words, if the angle 2915 between the two vectors 2912 and 2913 is less than 180 degrees, then the two pixels overlap. Referring now to FIG. 10H, and measuring the distance between pixel 2948 and the corner pixels of the other images 2920a-2920d, the shortest distance is between pixel 2948 and 2920d. The vector of that distance 2945 is in the opposite general direction as the vector 2944 along the top row of image 2944, so the distance represents a gap. In other words, if the angle 2949 between the two vectors 2944 and 2945 is greater than 180 degrees then the two pixels represent a gap.

Referring to FIGS. 10I and 10K, two adjacent scan sequences 2900a-2900d and 2904a-2904d are depicted. One means of measuring whether there is overlap or gap spacing is to measure the distances 2908a-2908d from one of the corner pixels of one image, for example P(FIRST-ROW, LAST-COLUMN) 2916 and each of the pixels in the same row, but opposite side of the image in all of the images in the adjacent row, for example P(FIRST-ROW, FIRST-COLUMN) 2920a-2920d. The shortest of those distances represents the spacing between adjacent images in adjacent rows. In the example of FIGS. 10I and 10K, that would be distance 2908b. The border pixel 2916 is considered to overlap with the adjacent scan sequence of images 2900a-2900b if the pixel is within the borders of the area 2953 described, in part, by the row of the closest image 2900b and the adjacent image 2900a. Referring now to FIGS. 10J and 10L, and measuring the distance between pixel 2948 and the corner pixels of the other images 2920a-2920d, the shortest distance is between pixel 2948 and 2920d. The border pixel 2948 is considered to have a gap with the adjacent scan sequence of images 2900a-2900b if the pixel is outside of the borders of the area 2955 described, in part, by the row of the closest image 2900d and the adjacent image 2900c.

Referring now to FIG. 10B and 10C, an alternative algorithm is employed wherein the volume subjected to successive scan sequences 80a-80m is transformed into the computed distribution of ultrasound scan image pixels based on the known position and computed orientation of the hand-held ultrasound probe assembly 30 for each scan sequence as described above in connection with FIG. 9. Using this alternative algorithm, the pixel density per unit volume (e.g., pixel density per cubical 1.0 cubic centimeter or pixel density per cubical 0.5 cubic centimeter unit volumes) can be computed for the included volume bounded by all successive scan sequences. By way of example and still referring to FIGS. 10B and 10C, the included volume 75 bounded by successive scan sequences 80d and 80e, would be subdivided into smaller unit volumes 79. The computed position of all pixels within the included volume 75 between scan sequences 80d and 80e would then be computed, based on the known position and computed orientation of the hand-held ultrasound probe assembly 30 during periods within each scan sequence, thereby allowing the computation of pixel density within each unit volume 79. The number of ultrasound scan pixels (as described above in connection with FIG. 9) contained in each unit volume 79 is computed and this number is compared to a predetermined Minimum Pixel Density number. If the computed pixel density within any unit volume 79 within the included volume 75 is less than the Minimum Pixel Density, then the operator is alerted at the end of the scan sequence that scan sequence just completed is incomplete and that it must be repeated including a display of instructions to improve the scanning method (e.g., reduce the spacing between the previous scan sequences and the present scan sequence to be repeated).

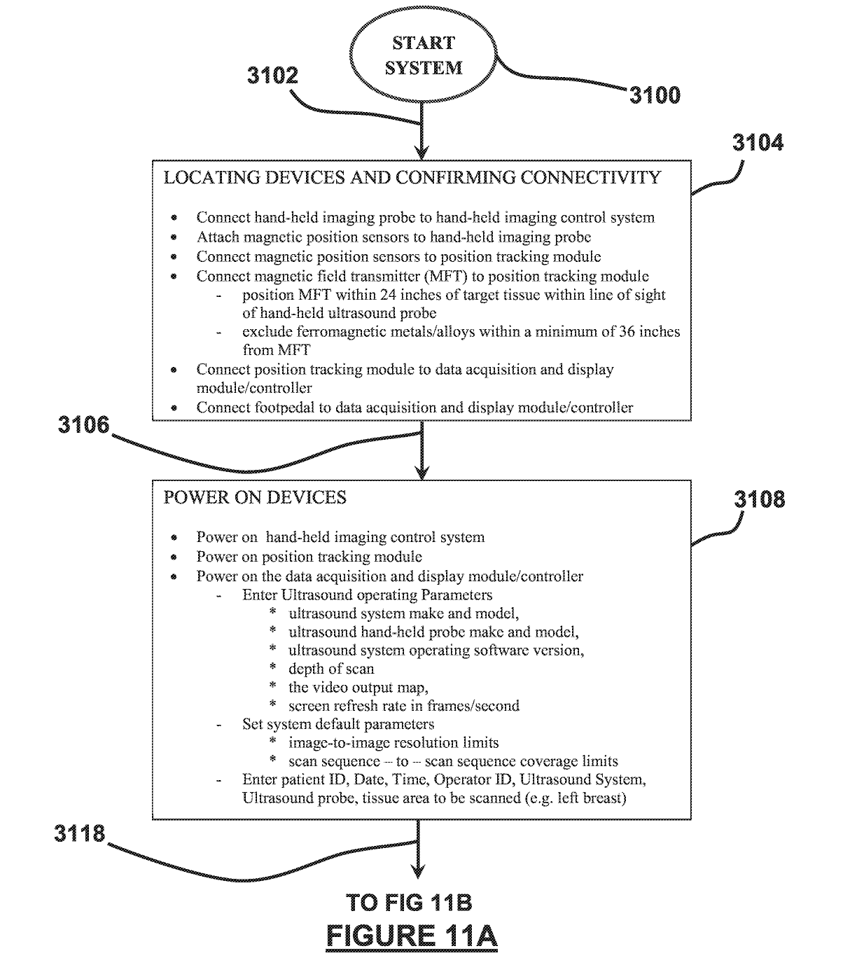

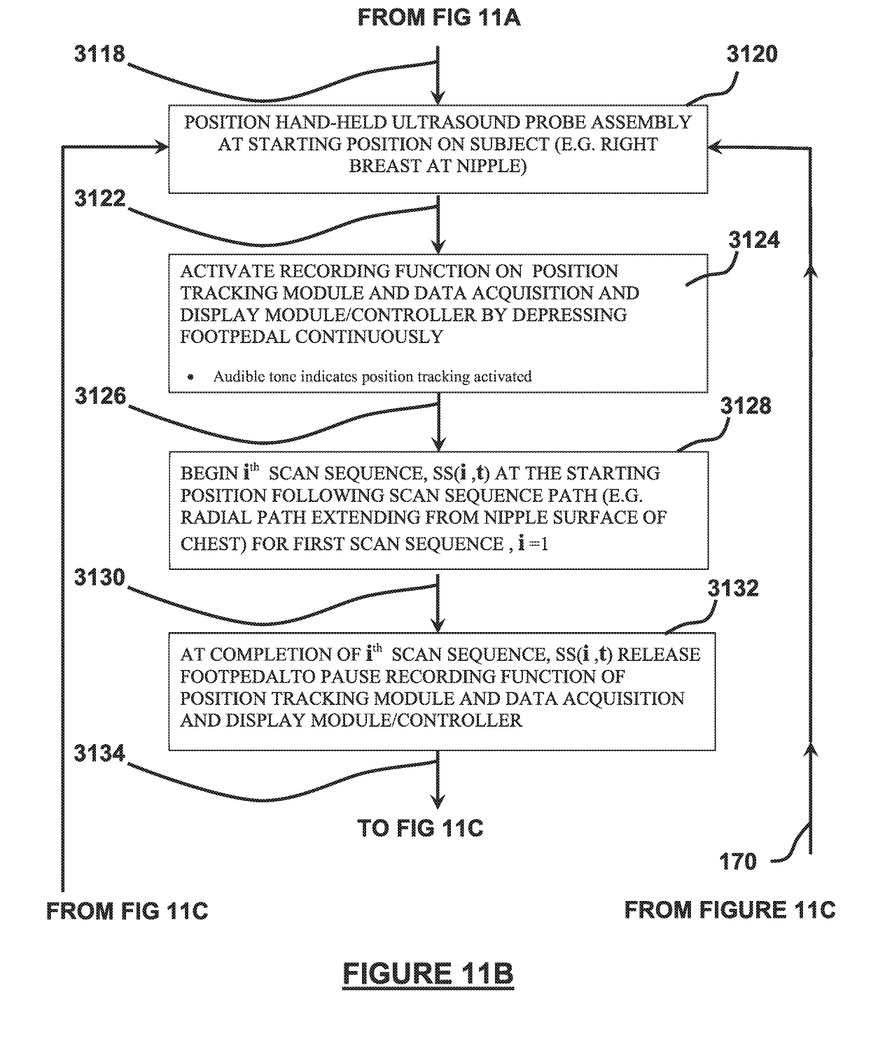

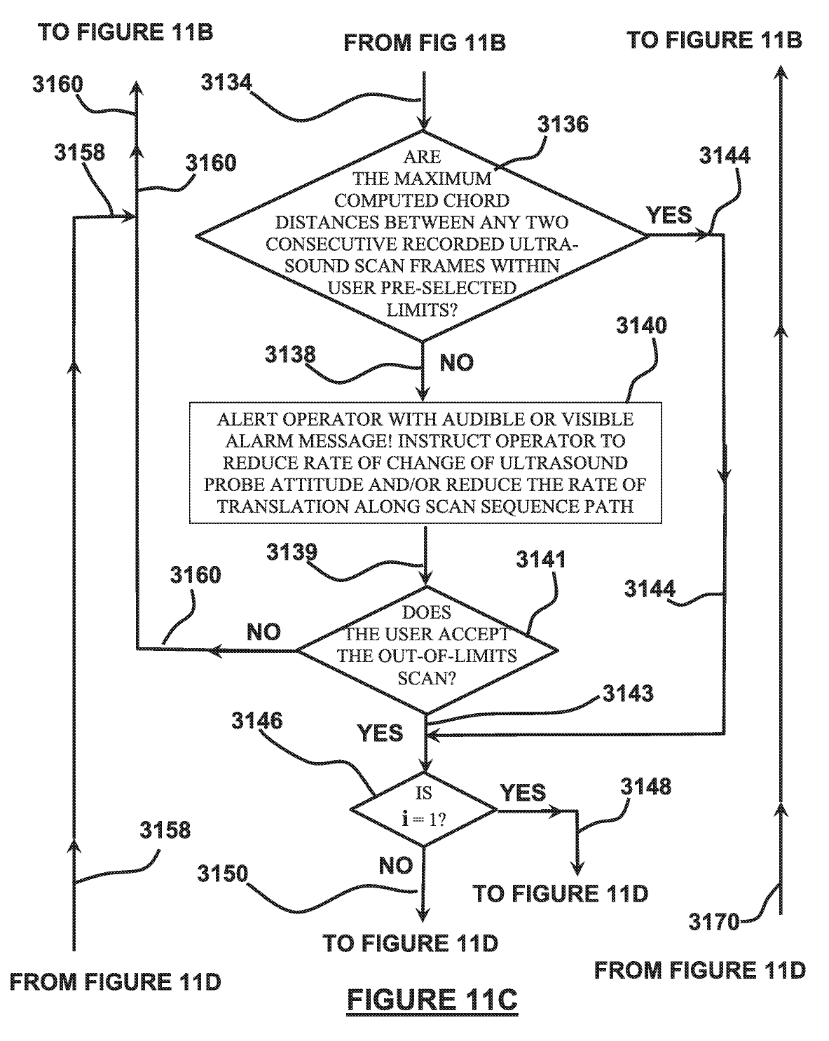

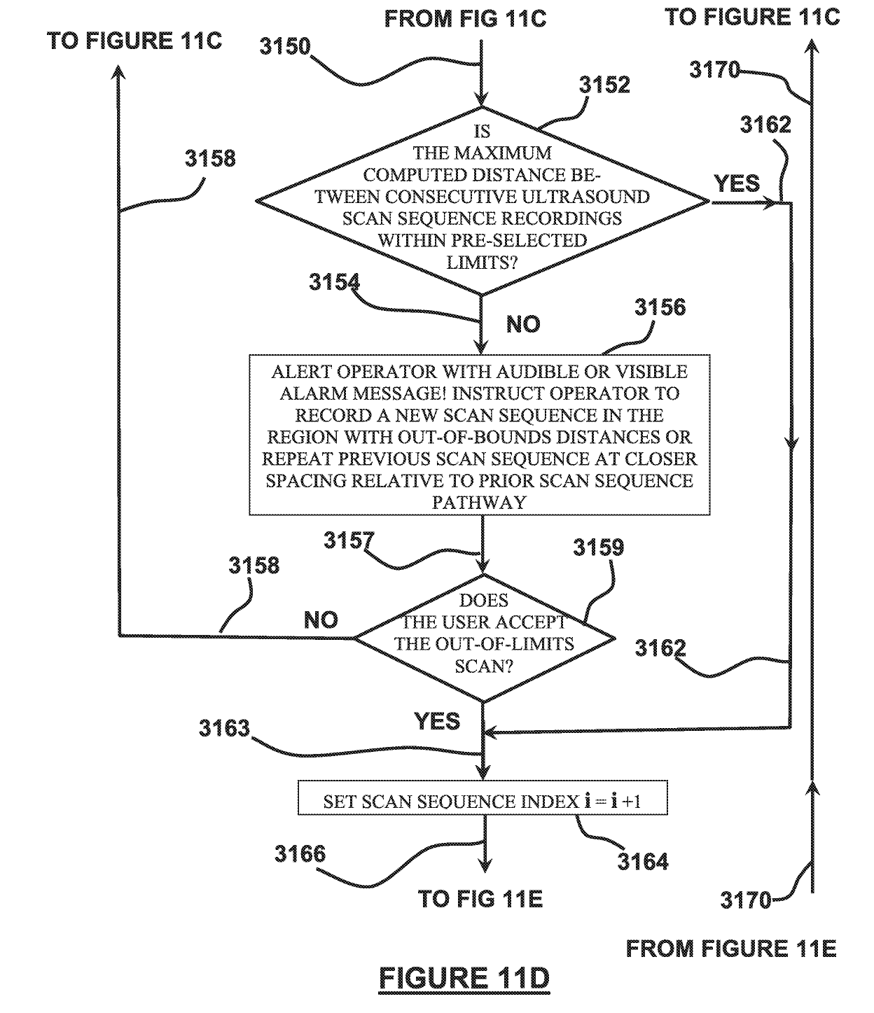

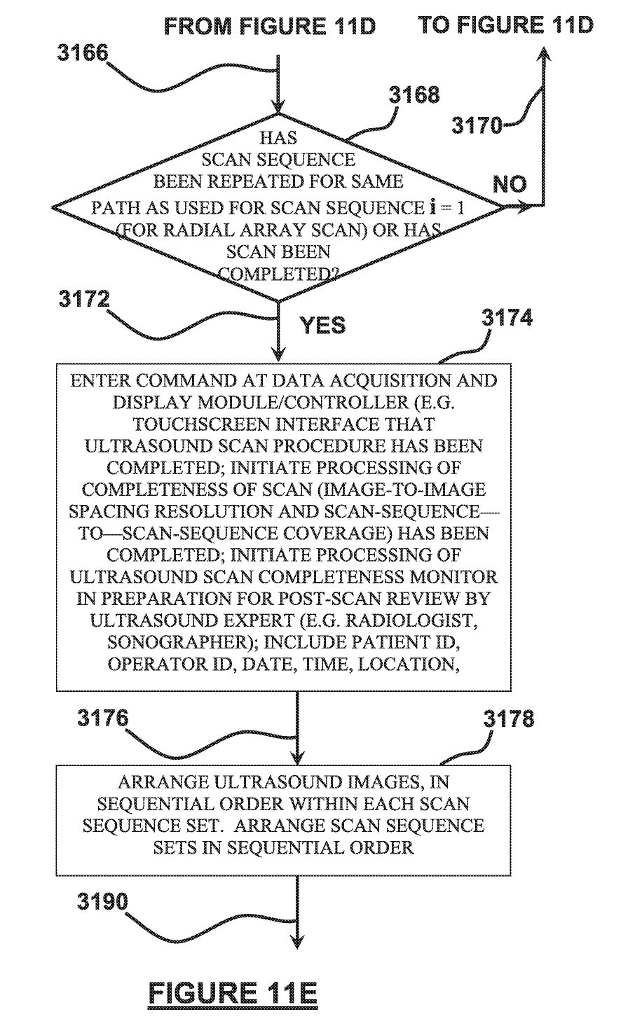

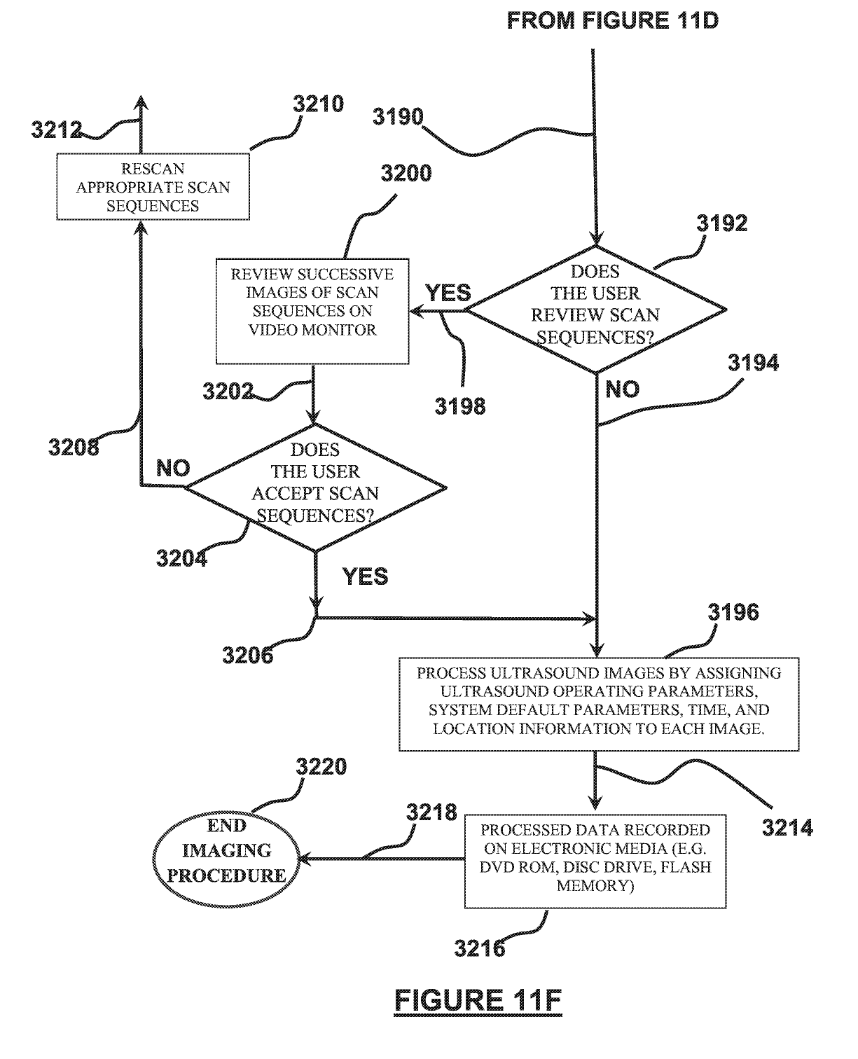

Turning now to FIGS. 11A through 11E, a flow chart describes one embodiment of the method and system of the present invention. Beginning as represented by symbol 3100 and continuing as represented by arrow 3102 to block 3104, connectivity of the components of the system is verified. The user must verify that the hand-held ultrasound imaging probe is connected to the ultrasound system, that the position sensors are attached to the hand-held ultrasound probe, that the position sensors are connected to the position tracking module, that the magnetic field transmitter (MFT) component of the position tracking module is within 24 inches of the targeted patient volume (e.g. the patient's breast), that there are no electromagnetic materials within 36 inches of the MFT (i.e., a requirement specifically related to the use of the Ascension Technology position detection product), that there is a clear line-of-sight between the expected positions of the ultrasound probe when it is on the targeted tissue volume and the position tracking module (i.e. a requirement specifically related to the use of visible detection technologies, such as is employed when an infrared camera tracks an visible register), that the that the position tracking module is connected to the data acquisition and display module/controller, and that the foot pedal is connected to the data acquisition and display module/controller.

Referring next to FIG. 11B, having completed the preliminary system set up and initialization steps, as represented by arrow 3118 to block 3120, the operator now proceeds to positioning the hand-held imaging probe at the starting position of the target tissue site on the patient (e.g., at the nipple of the right breast). Next, as represented by arrow 3122 to block 3124, the operator now proceeds to activate both the position tracking module and the associated data acquisition and display module/controller by depressing the foot pedal continuously during the entire period of each scan sequence performed using the hand-held ultrasound probe assembly with an audible tone issued and/or visible indicator confirming that the position sensing detection and recording function for the hand-held ultrasound probe assembly is currently active.

Once the position sensing detection and recording function has been activated, as represented by arrow 3126 to block 3128, the operator now proceeds to translate the hand-held imaging probe along the skin to begin the first of [i] scan sequences, SS[i,t] where i equals the number of scan sequences to be performed and t refers to the time period at which an ultrasound beam is emitted into the tissue and a returning acoustic signals are measured and recorded in what is referred to herein as an ultrasound scan “frame”. For the case of the first scan sequence (e.g., see scan sequence 80a in FIG. 10A), i is equal to 1.

Once the first scan sequence (i=1) is completed, as represented by arrow 3130 to block 3132, the operator releases the foot pedal to pause (i.e., to temporarily deactivate) the image recording function of the data acquisition and display module/controller. The time-stamped hand-held imaging probe position and computed orientation data acquired within the data acquisition and display module/controller is combined with the time-stamped ultrasound scan frames received from the ultrasound system to enable rapid computation of the image-to-image resolution of the scan sequence just completed. As represented by arrow 3134 to block 3136 as seen in FIG. 11B, the chord distances between any two successive scan frames are computed to determine if they are within pre-selected limits as illustrated with regard to FIG. 8B discussed above.

Still referring to FIG. 11B, an alternative embodiment of the present invention can be substituted at block 3136, which utilizes the imaging scan pixel density within the swept volume of the complete scan sequence as was described with regard to FIG. 9. In this alternative algorithm, the time-stamped hand-held imaging probe position and computed orientation data acquired within the data acquisition and display module/controller is combined with the time-stamped imaging scan frames received from the ultrasound system to enable rapid computation of the completeness of the scan sequence just completed. However, rather than computing the distances between successive scan frames, the pixel density within unit volumes within the swept volume are computed to determine if the computed pixel density is less than the preselected Minimum Pixel Density value.

Still referring to FIG. 11C, using either of the above two algorithms (i.e., scan frame distance based computations or volumetric pixel density within unit volumes of the swept volume), if the predetermined requirement is not met (i.e., maximum allowed distance between scan frames is exceeded or the minimum required pixel density is achieved for all unit volumes), then block 3140 is reached via arrow 3138. As seen in block 3140, an audible alarm and visual error message is issued to instruct the operator that the scan failed to comply with the minimum user requirements for frame-to-frame resolution. As represented by arrow 3139 and block 3141, the user is queried as to whether he or she wishes to accept this scan sequence, SS(i), which does not meet the user-defined minimum limits of frame-to-frame resolution. If the operator does not choose to accept the scan sequence SS(i), which does not meet the user-defined minimum limits of frame-to-frame resolution, then, as represented by arrow 3160 to block 3120, the operator repeats the scan sequence previously performed but determined to be incomplete due to the failure of the frame-to-frame resolution to meet the minimum user-defined requirements. If the user chooses to accept the scan sequence SS(i), which does not meet the user-defined minimum limits of frame-to-frame resolution, then block 3146 is reached via arrow 3143.

Still referring to FIG. 11C, using either of the above two algorithms (i.e., scan frame distance based computations or volumetric pixel density within unit volumes of the swept volume), if the user chooses predetermined requirement is met (i.e., maximum allowed distance between scan frames or minimum required pixel density), then block 3146 is reached via arrow 3144. If this is the first scan sequence (i.e., i=1), then the computation of distances between successive scan sequences (i.e., the maximum distance between ultrasound scan frames in scan sequence 80d and 80e as exemplified in FIG. 10B) is bypassed thereby proceeding to block 3164 via arrow 3148. In block 3164, the scan sequence index, is increased by the number 1. For this example description, the value of i was 1 and is now 2.

Referring now to FIG. 11D, as represented by arrow 3166 and block 3168, a computation is performed to determine if the scan sequence just completed is essentially the same as the initial scan sequence performed or, alternatively, if the last scan sequence has been performed for the target tissue volume. For the case of the human breast with successive radially oriented scan sequences progressing in a circular pattern as seen in FIG. 10A, the last scan sequence is obtained when the first scan sequence is essentially repeated. Alternatively, if the target tissue being scanned involves a rectangular pattern of successive scan sequences, the operator designates on the data acquisition and display module/controller that the last scan sequence has been performed. If the scan sequence just completed is not the last scan sequence required for the ultrasound examination, proceed as represented by arrow 3170 to block 3120 to initiate sequence of steps for next scan sequence.

Returning to block 3146 in FIG. 11C, if scan sequence i is greater than 1, then one of the above two algorithms (e.g., either computation of distance between two successive scan sequences or volumetric pixel density within unit volumes of the included volume between successive scan sequences) are used to determine the edge-to-edge coverage of the two successive scan sequences just completed as specified in block 3152. If the predetermined requirement is met (i.e., maximum allowed distance between the adjacent edges of scan frames in successive scan sequences is not exceeded or the pixel density in any unit volume is not less than the minimum required pixel density), then block 3164 is reached via arrow 3162. If the predetermined requirement is not met (i.e., maximum allowed distance between adjacent edges of scan frames in successive scan sequences is exceeded or the pixel density in any unit volume is less than the minimum required pixel density), then block 3156 is reached via arrow 3154. As seen in block 3156, an audible alarm and visual error message is issued to instruct the operator to determine that the coverage, as defined by the user-defined edge-to-edge spacing of adjacent edges in successive scan sequences, or the user-defined pixel density in any unit volume is less than the required pixel density, has not been met. Then block 3159 is reached via arrow 3157. The user is queried regarding whether he or she wishes to accept , scan sequence, SS(i), is to be accepted that the coverage, as defined by the user-defined edge-to-edge spacing of adjacent edges in successive scan sequences, or the user-defined pixel density in any unit volume is less than the required pixel density, has not been met. If the user chooses even though the coverage, as defined by the user-defined edge-to-edge spacing of adjacent edges in successive scan sequences, or the user-defined pixel density in any unit volume is less than the required pixel density, has not been met, to accept the scan sequence, SS(i), then block 3164 is reached via arrow 3163. If the user chooses not to accepted scan sequence, SS(i), because that the coverage, as defined by the user-defined edge-to-edge spacing of adjacent edges in successive scan sequences, or the user-defined pixel density in any unit volume is less than the required pixel density, then the scan sequence is repeated at a closer spacing relative to the prior scan sequence pathway. As represented in FIG. 11D, FIG. 11C, and FIG. 11B, arrow 3158 joins arrow 3160 to block 3120, wherein the operator repeats the scan sequence previously performed since it was determined to be incomplete due to regions of the target tissue not being included in the series of ultrasound scan frames just obtained.

Throughout the hand-held imaging procedure, the progression of scan sequences is shown on the screen of display 3 of the data acquisition and display module/controller 40 with the sequential scan index, i identified adjacent to each completed scan sequence in a manner similar to the illustration in FIG. 10A.

Returning to block 3174 of FIG. 11E, at the completion of the hand-held image scanning procedure and the verification that the target tissue ultrasound scans included all tissue within the target tissue volume (i.e., a complete diagnostic ultrasound scan was achieved), then the processing of the ultrasound scan frames is performed within the data acquisition and display module/controller. Arrow 3176 follows to block 3178, wherein the scanned images are arranged in a sequential order (i.e., progressing with elapsed time during procedure). In this step, the image data are captured and converted to a format that is easily stored and compatible with a viewer.

Referring to FIG. 11E and FIG. 11F, arrow 3190 joins block 3192 in which the user is queried regarding whether he or she wishes to view the scan sequences before processing the data and saving the procedure study. The viewer allows playback of the scanned images by the expert reviewer (e.g., radiologist) in a manner that is optimized for screening for cancers and other anomalies. If the user chooses to forego review, then arrow 3194 joins block 3196.

Still referring to FIG. 11F, if the user does choose to review the scans then arrow 3198 proceeds to 3200, in which the scan sequence images are displayed on a video monitor, such as a digital computer monitor. After review of the scan sequences, the system queries the user whether he or she wishes to accept the study. As depicted by arrow 3204 proceeding to join arrow 3194, which proceeds to block 3196, the images are processed. If the user chooses to not accept the images then a rescanning sequence is initiated as depicted by arrow 3208 proceeding to block 3210.

Still referring to FIG. 11F, the complete set of sequenced image frames are assigned patient, ultrasound instrument information, time, and location information as depicted in block 3196. The processed data is then stored on electronic media, such as a DVD ROM, disc drive, or flash memory drive). This process is depicted by arrow 3214 proceeding to block 3216. The DVD-ROM (or other suitable recording media) is physically transferred from the data acquisition and display module/controller to the expert (e.g., radiologist) for subsequent analysis and evaluation of the diagnostic ultrasound data with the confidence that the entire target tissue volume has been included in the supplied data recording. This last step defines the end of the diagnostic examination procedure for a particular patient. After the data is stored the image procedure is concluded as depicted by arrow 3218 proceeding to block 3220.

In addition to mapping the three-dimensional position of the pixels recorded from a set of two-dimensional images, the method, apparatus and system of some described embodiments performs a pixel density calculation to provide an objective characterization of the resultant image set to determine whether that spacing in the Z direction is sufficient to provide an accurate and complete three-dimensional image of the targeted tissue volume (e.g., the human female breast). By way of example, each of the pixels in each ultrasound scan-derived two-dimensional image, i are specified by a unique set of coordinates X{i,j} and Y{i,j} in two-dimensional space. When two adjacent two-dimensional images i and i+1 are combined to form a three-dimensional volume, then the position of each pixel is transformed into three-dimensional space and can be defined by the three Cartesian coordinates Xij, Yij and Zij.

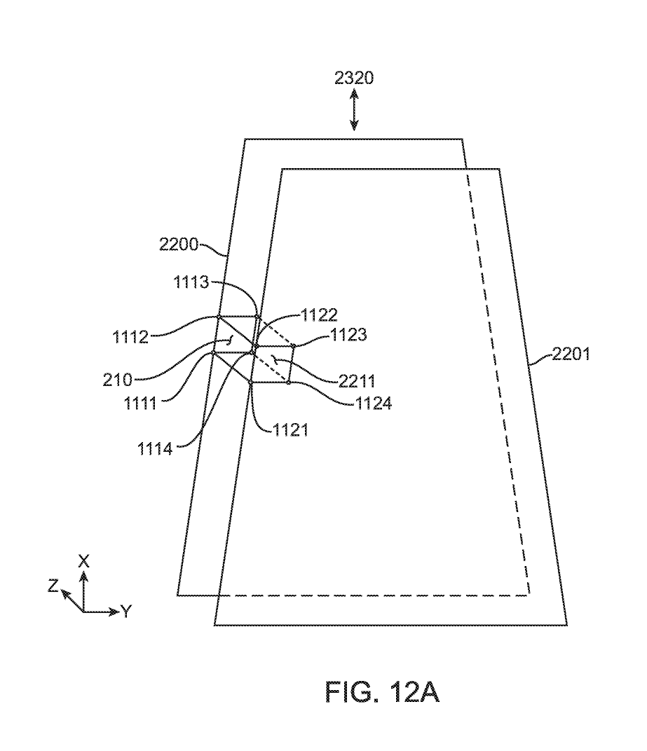

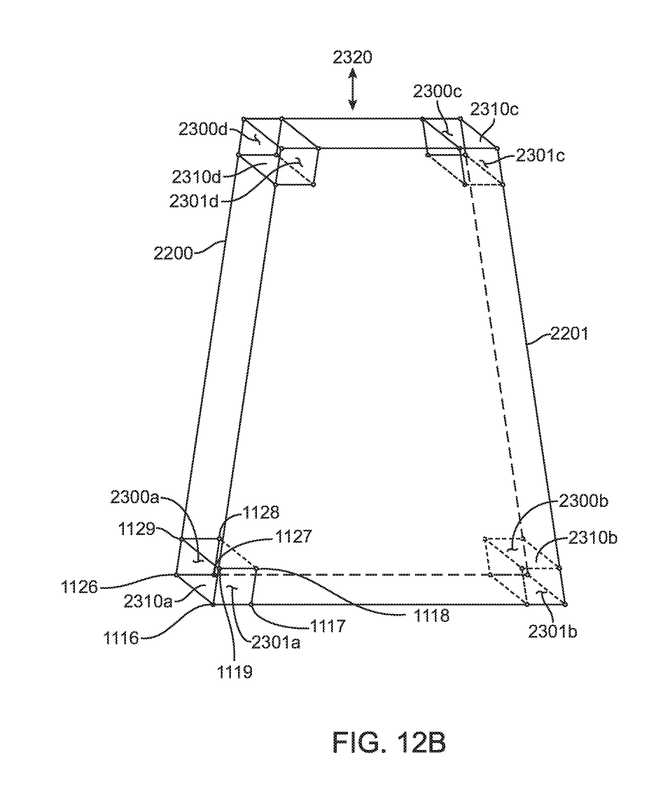

Continuing with this this example and referring to FIG. 12A, assume that the overall volume circumscribed by any two adjacent two-dimensional scans is subdivided into smaller component volumes. By way of example, said smaller component volumes have two opposite square side faces measuring 2 mm×2 mm and are defined, as seen in FIG. 12A, by the coordinates listed below. To facilitate the notation of XYZ coordinates at the boundaries of the example component volume, the physical spacing between sequential two-dimensional ultrasound scan images 2200 and 2201 has been significantly increased and is not drawn to scale relative to the overall dimensions of the ultrasound scan regions 2200 and 2201.

Coordinates of Square Side Faces on two-dimensional image 2200:
X₁₁Y₁₁Z₁₁ (1111), X₁₂Y₁₂Z₁₂ (1112), X₁₃Y₁₃Z₁₃ (1113), X₁₄Y₁₄Z₁₄ (1114),

Coordinates of Square Side Faces on (i+1)th two-dimensional image 2201:

X₂₁Y₂₁Z₂₁ (1121), X₂₂Y₂₂Z₂₂ (1122), X₂₃Y₂₃Z₂₃ (1123), X₂₄Y₂₄Z₂₄ (1124)

Continuing with this example, the maximum spacing between the square 2 mm×2 mm faces on adjacent two-dimensional images 2200 and 2201 for the first component volume is determined by comparing the following four distances along the Z axis:
{Z₁₁−Z₂₁}, {Z₁₂−Z₂₂}, {Z₁₃−Z₂₃}, {Z₁₄−Z₂₄}

For this example, assume that the maximum distance between the four corners of the squares 2210 and 2211 in FIG. 12A is {Z₁₄−Z₂₄}. Then the computed first component volume is the product of the unit area, A and the maximum spacing between the square faces 2210 and 2211 (2 mm×2 mm for this example):

First Component Volume=A*{Z₁₄−Z₂₄} EQ. 2

Continuing with this example and still referring to FIG. 12A, the First Component Volume Pixel Density for the First Component Volume is given by dividing the combined total number of pixels within the 2 mm×2 mm areas, A on faces 2210 and 2211 on the two sequential two-dimensional images (e.g., 400 pixels on each image for a combined total of 800 pixels for two sequential images) by the First Component Volume given in Equation 3 as follows:

First Component Volume Pixel Density=(Total No. of Pixels in both Unit Areas)÷(First Component Volume) EQ. 3

Referring now to FIG. 1 and FIG. 12A and continuing with this example, the computed First Component Volume Pixel Density obtained in Equation 3 is compared with a predetermined Minimum Allowed Volumetric Pixel Density, which is selected to ensure that all regions within the targeted tissue volume are included in the ultrasound scan. The above example process is repeated (a) for each component volume defined by the boundaries of two sequential two-dimensional images 2200 and 2201 and (b) for all pairs of sequential two-dimensional images acquired during a screening procedure. If any sequential pair of two-dimensional ultrasound scans results in a Component Volume Pixel Density which is less than the Minimum Allowed Volumetric Pixel Density, then a warning is displayed on the data acquisition and display module/controller 40 so that the operator can repeat the ultrasound scan sequence just completed to increase the pixel density to meet the requirements of the predetermined Minimum Allowed Volumetric Pixel Density. By this process, a complete ultrasound screening is assured which includes all tissue volumes within the targeted tissue region.

Another embodiment of the present invention utilizes the geometrical relationship of any two sequential ultrasound scan images to reduce the number of component volumes that need to be analyzed to determine if [a] the maximum spacing limit between sequential ultrasound scan images has been exceeded and/or [b] the minimum pixel density in a component volume has not been achieved. Referring now to the example in FIG. 12B, two sequential two-dimensional ultrasound scan images 2200 and 2201 are shown in a spaced apart relationship with vector 2320 referring to the direction of transmitted and reflected ultrasound signals emanating from and received by the hand-held ultrasound probe. To facilitate the notation of XYZ coordinates at the boundaries of the example component volumes, the physical spacing between sequential two-dimensional ultrasound scan images 2200 and 2201 has been significantly increased and is not drawn to scale relative to the overall dimensions of the ultrasound scan regions 2200 and 2201.

Each two-dimensional ultrasound scan image, e.g., scan images 2200 and 2201, can be assumed to take the geometric form of a flat planar surface. In addition, since any two sequential two-dimensional ultrasound scan images are acquired within a very short time period, the boundary of the two-dimensional scan image (e.g., scan image 2200) is registered with and can be projected onto the boundary of the (i+1)^{th} two-dimensional scan image (e.g., scan image 2201). As a result of the registration of the boundaries of any two sequential two-dimensional ultrasound scan images and their planar geometry, only those component volumes located at the four “corners” of the pair of sequential two-dimensional ultrasound scan images, as seen in FIG. 12B need to be analyzed to determine if [a] the maximum spacing limit between sequential ultrasound scan images has been exceeded and/or [b] the minimum pixel density in a component volume has not been achieved.

By way of example and still referring to FIG. 12B, the Cartesian coordinates for component volume 2310a are shown in detail. Said component volume 2310a is comprised of two isosceles trapezoids 2300a and 2301a corresponding to end faces of the component volume 2310a located at one of four corners of the planar two-dimensional ultrasound scan images 2200 and 2201, respectively. The coordinates of 2300a are X₂₈Y₂₈Z₂₈ (1128), X₂₉Y₂₉Z₂₉ (1129), X₂₆Y₂₆Z₂₆ (1126), X₂₇Y₂₇Z₂₇ (1127). The coordinates of 2301a are X₁₆Y₁₆Z₁₆(1116), X₁₇Y₁₇Z₁₇(1117), X₁₈Y₁₈Z₁₈(1118), X₁₉Y₁₉Z₁₉ (1119), The Cartesian coordinates at each of the four corners of each of the isosceles trapezoids defining the component volume 2310a are used to determine the maximum spacing among the four Z-axis distances {Z₁₆−Z₂₆, Z₁₇−Z₂₇, Z₁₈−Z₂₈, Z₁₉—Z₂₉} between this pair of isosceles trapezoids 2300a and 2301a. This same procedure is next used to determine the maximum spacing between among the four Z-axis distances between pairs of isosceles trapezoids 2300b and 2301b, 2300c and 2301c and 2300d and 2301d corresponding to component volumes 2310b, 2310c and 2310d, respectively, as seen in FIG. 12B. These maxima for each of the four isosceles trapezoid pairs are next compared to determine which component volume among the four component volumes 2310a, 2310b, 2310c, or 2310d contains the maximum inter-scan image spacing along the Z-axis. That component volume 2310 containing the maximum inter-scan image spacing along the Z-axis is then used to determine if the requirements for maximum allowed inter-scan image spacing and/or minimum required pixel density have been achieved. If these predetermined requirements are not met, the operator is promptly alerted (e.g., with a visual cue indicating that the just completed ultrasound scan was not properly performed along with specified step(s) to correct the detected deficiency in the ultrasound scan.

By this novel method, the described embodiments greatly reduces the computation time required to assure that each subsequent two-dimensional ultrasound scan image meets the requirements for maximum allowed spacing and/or minimum required pixel density and that the operator can be alerted immediately after each scan path has been completed.

When the two-dimensional ultrasound scan-derived images are being presented in sequence, the greater the spacing between sequential scans (i.e., along the Z-axis as seen in FIG. 12A), the more compromised the ability of the clinician reviewing the screening images to accurately identify and characterize the lesion. By way of example, if the images are being presented at 15 frames per second, which is not unusual since the viewer will be accustomed to viewing a succession of still images as rapidly as 30 frames per second in standard video presentations, then a 1 mm spacing between two sequential, adjacent two-dimensional images would represent a presentation duration of 0.33 seconds of any unusual structure. In contrast, the case of a 3 mm spacing between two sequential, adjacent two-dimensional images would represent a presentation duration of only 0.07 seconds of any unusual structure due to the larger spacing between images. Since the brain has the capability to automatically detect unusual changes in the visual environment, a method, apparatus and system for displaying a “normal” image or a series of “normal” images, followed by an “unusual” image or a series of “unusual” images, will induce an involuntary recognition response (see Pazo-Alvarez, P., et. al., Automatic Detection of Motion Directed Changes in the Human Brain 2004. European Journal of Neuroscience; 19: 1978-1986). Studies with motion picture presentation suggest that frame rates slower than 15 frames/second are perceived less as motion, and more as individual images (see Read, P., et. al., Restoration of Motion Picture Film 2000. Conservation and Museology, Butterworth-Heinemann, ISBN 075062793X: 24-26). Thus, the presentation of a single frame of a random structure for the minimal period of time is more prone to being “missed” by the clinician/reviewer than the presentation of a series of sequential images of that structure over a longer period of time.

Minimizing the time duration of the reviewing process while maximizing the ability to recognize abnormalities within the video presentation of the ultrasound screening results is of primary importance to the clinician to avoid fatigue and maximize the efficient use of the clinician's time. The ultrasound scanning-derived image recording is time-based, with the images obtained in a temporally uniform manner This approach can present several problems. First, if the image spacing varies from one part of the scan to the next, then the ability to present the images in a spatially uniform manner is compromised. One portion may have images spaced on 0.01 mm centers while another may have them spaced on 1 mm centers. If the information recorded during the portion where images were recorded at 0.01 mm centers will take 10 times longer to display the same subset of swept volume of scan sequence as does the portion where images were recorded on 0.1 mm centers. When seeking to detect abnormalities on the order of 5 mm, it can be argued that there is no more real information presented in the 0.01 mm-center scans than there is in the 0.1 mm-center scans. The portion with the more closely spaced images may represent a reduction in viewer efficiency, not an increase in procedure efficacy.

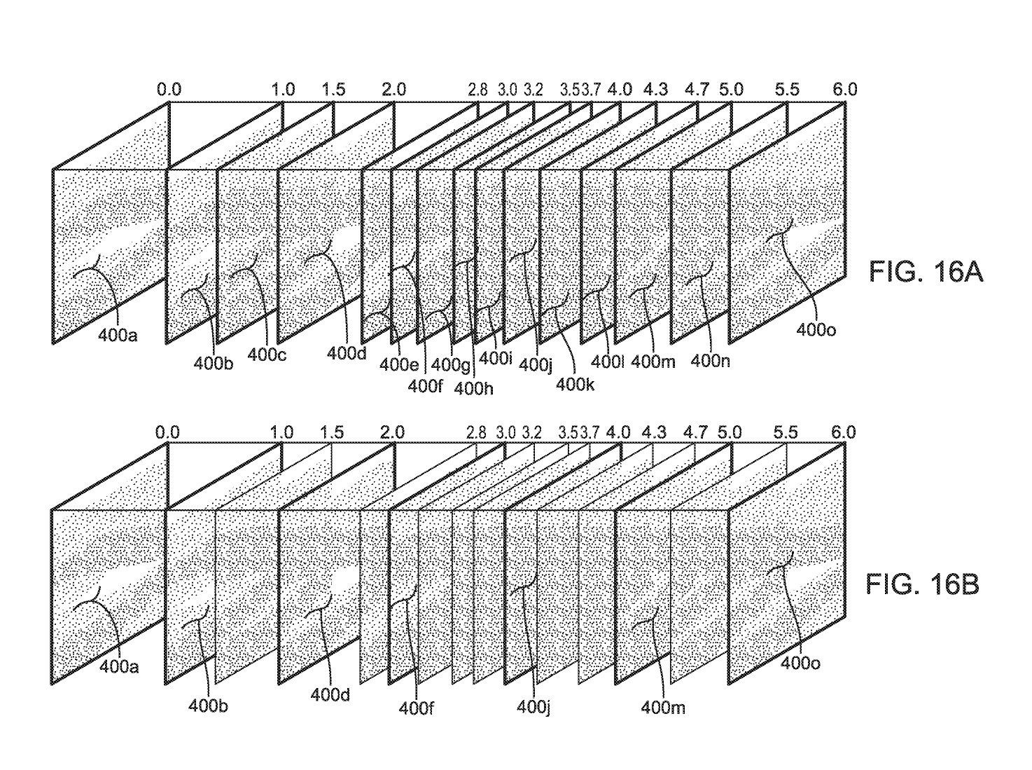

Another embodiment of the present invention is seen in FIGS. 16A-16B and includes analyzing the complete data set from the ultrasound screening procedure to identify those two-dimensional scan images 400a-400o that are separated by a function of the translational speed of the ultrasound probe during the scanning procedure and the image recording rate of the data acquisition and control module. In one embodiment, those images that are separated by a Z-axis spacing close to the predetermined minimum spacing interval are saved while any additional two-dimensional scan images located between a pair of properly spaced two-dimensional scan images, consequently being separated by a spacing interval much less than the predetermined minimum spacing interval, are excluded from the final video presentation of the ultrasound scanning procedure. In the way of example, as described in FIG. 16A, if, because of variations in the translational speed during the scanning procedure, images are recorded at 0.0 mm, 1.0 mm, 1.5 mm, 2.0 mm, 2.8 mm, 3.0 mm, 3.2 mm, 3.5 mm, 3.7 mm, 4.0 mm, 4.3 mm, 4.7 mm, 5.0 mm, 5.5 mm, and 6.0 mm centers, and if the preferred image spacing is 1.0 mm, then only those images recorded at 0.1 mm, 1.0 mm, 2.0 mm, 3.0 mm, 4.0 mm, 5.0 mm, and 6.0 mm will be displayed (that is, 400a, 400c, 400d, 400f, 400j, 400m, and 400o). The other images, 8 of the 15 recorded images, will not be displayed, reducing the viewing time by more than 50% (FIG. 16B). As a result of this embodiment of the present invention, the clinician is able to review the minimum number of images with essential visual information content. This method for post-processing the ultrasound screening data, with predetermined image spacing, provides a temporally and spatially uniform presentation.

Another embodiment of this present invention, also seen in FIGS. 16A-16B includes analyzing the complete data set from the ultrasound screening procedure to identify the spacing between each pair of adjacent scan images and to present those images in a spatially consistent manner, rather than a temporally consistent manner, as is the custom with most presentations of video images. The presentation of images is provided as a function of sweep volume and the dwell time for each image is determined as a function of the spacing between adjacent images. In the way of example, as described in FIG. 16A, if, because of variations in the translational speed during the scanning procedure, images are recorded at 0.0 mm, 1.0 mm, 1.5 mm, 2.0 mm, 2.8 mm, 3.0 mm, 3.2 mm, 3.5 mm, 3.7 mm, 4.0 mm, 4.3 mm, 4.7 mm, 5.0 mm, 5.5 mm, and 6.0 mm centers, and if the preferred image spacing is 1.0 mm/sec, then the dwell time, or the time the image is displayed before the next sequential image is displayed for 400a is 1.0 sec because the distance between 400a and 400b is 1.0 mm. The dwell time is calculated by dividing the distance between frames by the desired spatial presentation rate [1.0 mm/(1.0 mm/sec)]. In like manner the dwell time for 400b is 0.5 sec because the distance between 400b and 400c is 0.5 mm [0.5 mm/(1.0 mm/sec)]. In like manner the dwell times for 400c is 0.8 sec, for 400d is 0.2 sec, for 400e is 0.2 sec, for 400f is 0.3 sec, for 400g is 0.2 sec, for 400h is 0.3 sec, for 400i is 0.3 sec, for 400j is 0.4 sec, for 400k is 0.3 sec, for 400l is 0.5 sec, and for 400m is 0.5 sec. No dwell time is listed for 400o in this example because there is no sequential frame following 400o.

Referring to FIG. 1 and FIGS. 16A-16B, if the user varies his or her speed during the scan sequence, then there will be variable spacing in the images 400 that could be recorded, if those images 400 were recorded at regular time intervals. The position tracking module 22 and the data acquisition and display module/controller 40 poll the location of the hand-held imaging probe 14 to which the plurality of position sensors 32a, 32b and 32c are affixed at time intervals that are more frequent than the expected recording time interval to determine when the hand-held imaging probe 14 to which the plurality of position sensors 32a, 32b and 32c are affixed is at a location which would represent an acceptable spacing, regarding the previously recorded image 400. When the hand-held imaging probe is at the appropriate space, the data acquisition and display module/controller 40 will record an image. For example, in FIGS. 16A-16B, if images 400a-400o represent the location of the hand-held imaging probe 14 to which the plurality of position sensors 32a, 32b and 32c are affixed at 0.1 sec intervals, then the data acquisition and display module/controller 40 would only record an image at 0.0 seconds 400a (when the hand-held imaging probe 14 to which the plurality of position sensors 32a, 32b and 32c are affixed is at its initial location), another image at 0.1 sec 400b (when the hand-held imaging probe 14 to which the plurality of position sensors 32a, 32b and 32c are affixed is 1.0 mm past the previously recorded image, or at 1.0 mm), another image at 0.3 sec 400d (when the hand-held imaging probe 14 to which the plurality of position sensors 32a, 32b and 32c are affixed is 1.0 mm past the previously recorded image, or at 2.0 mm), another image at 0.5 sec 400f (when the hand-held imaging probe 14 to which the plurality of position sensors 32a, 32b and 32c are affixed is 1.0 mm past the previously recorded image, or at 3.0 mm), another image at 0.9 sec 400j (when the hand-held imaging probe 14 to which the plurality of position sensors 32a, 32b and 32c are affixed is 1.0 mm past the previously recorded image, or at 4.0 mm), another image at 1.2 sec 400m (when the hand-held imaging probe 14 to which the plurality of position sensors 32a, 32b and 32c are affixed is 1.0 mm past the previously recorded image, or at 5.0 mm), and another image 1.4 sec 400o (when the hand-held imaging probe 14 to which the plurality of position sensors 32a, 32b and 32c are affixed is 1.0 mm past the previously recorded image, or at 6.0 mm). The result would be 7 stored images which could be played back at almost half the time as would be required if all images which could have been recorded at regular time intervals were recorded.

Some embodiments described provide for the control of the imaging recording process by taking into consideration several factors during the scanning process. For example, these factors include image-to-image spacing, angular position of the probe, and scan-to-scan spacing. This allows the images to be recorded with uneven or non-constant spacing between one or more images. Uneven or non-constant spacing is often the result of variable translation speed as the operator moves the probe across a target region. Variable speed creates images of varying distances from one another. Some embodiments allow the operator to vary the speed of scanning while still ensuring adequate resolution and coverage of the scanned images. This can be accomplished by maintaining a minimum image-to-image distance, minimum scan-to-scan distance, or minimum pixel density.

As a further example, if the user varies his or her translational speed during a process so that the plurality of recorded images 400a-400o (see FIGS. 16A-16B), each having its own unique location identifier information, are spaced unevenly, the system and method can reduce the review time by calculating which of those images provide useful information and should be displayed during the review process, and which, because they are so closely spaced to the previous or following image, should not be displayed. By way of example, if the user wishes to review the 6 mm of tissue described in FIGS. 16A-16B, and the system has stored the 14 images 400a-400o, then the system and method may perform calculations using one or more microprocessors to determine which of the recorded images is closest to the desired spacing. Again by example, if the desired spacing is 1.0 mm, then only images 400a, 400b, 400d, 400f, 400j, 400m, and 400o are required to provide the desired resolution. The system can choose, through a logical argument which chooses only those images closest to the desired spacing parameters, to not display images 400c, 400e, 400g, 400h, 400i, 400k, 400l, and 400n.

If the user varies his or her translational speed during a process so that the plurality of recorded images 400a-400o, each having its own unique location identifier information, are spaced unevenly, the system and method can reduce the review time by calculating how long each of those images should be displayed during the review process, and which, because they are so closely spaced to the previous or following image, should not be displayed. By way of example, if the user wishes to review the 6 mm of tissue described in FIG. 16A, and the system has stored the 14 images 400a-400o described in FIG. 16A, then the system and method may perform calculations to determine how long to display each image, depending on the speed at which the reviewer wants to translate, from a virtual point of view, through the tissue. Again by example, if the desired spacing in FIG. 16 the space between image 400a and image 400b is 1.0 mm. If the reviewer wishes to review the images at 10 mm/sec, then the amount of time image 400a would be displayed before image 400b is displayed is 0.1 sec (1.0 mm/(10 mm/sec)). If the distance between image 400b and 400c is 0.5 mm, then the amount of time image 400b would be displayed before image 400c is displayed is 0.05 sec (0.5 mm/(10 mm/sec)). This process would be applied to all of the images so that the associated dwell time, or time for which each images is displayed is 400a=0.1 sec, 400b=0.05 sec, 400c=0.05 sec, 400d=0.08 sec, 400e=0.02 sec, 400f=0.02 sec, 400g=0.03 sec, 400h=0.02 sec, 400i=0.03 sec, 400j=0.03 sec, 400k=0.04 sec, 400l=0.04 sec, and 400m=0.05 sec. The total review time for this sequence is 0.56 sec. If the images were reviewed at 0.1 frames per second, as would be suggested from the spacing of images 400a and 400b, then the review time of the entire set of images would be 1.3 sec.

Other embodiments described provide for systems and methods for providing a speeded review time by limiting the number of images recorded. If an operator varies his or her speed during the scan process and the images are recorded at regular time intervals, then the recorded images will have irregular spacing. It is not necessary, however, that the system records the images at regular time intervals. The system may determine when to record the image by calculating where the image is in space, rather than as a function of time. By way of example, if the system recorded 19 images in one second, with the Z-plane location of those images being 0.0 mm recorded at 0.0 sec, 0.7 mm recorded at 0.1 sec, 0.9 mm recorded at 0.2 sec, 1.9 mm recorded at 0.3 sec, 2.5 mm recorded at 0.4 sec, 2.8 mm recorded at 0.5 sec, 3.6 mm recorded at 0.6 sec, 3.7 mm recorded at 0.7 sec, 4.0 mm recorded at 0.8 sec, 4.7 mm recorded at 0.9 sec, 5.1 mm recorded at 1.0 sec, 5.6 mm recorded at 1.1 sec, 6.6 mm recorded at 1.2 sec, 7.0 mm recorded at 1.3 sec, 7.6 mm recorded at 1.4 sec, 8.2 mm recorded at 1.5 sec, 8.5 mm recorded at 1.6 sec, 9.5 mm recorded at 1.7 sec, and 10.0 mm recorded at 1.8 sec, then the time to record those 19 images is 1.8 sec and the time to review them would be 1.8 sec at 10 frames per second. If the system only recorded images when they were at the desired spacing, then the review time and the image storage requirements would be lessened. By way of the above example, the probe is at 0.0 mm at 0.0 sec, it is at 1.0 mm at approximately 0.21 sec, it is at 2.0 mm at approximately 0.3167 sec, it is at 3.0 mm at approximately 0.5125 sec, it is at 4.0 mm at 0.8 sec, 5.0 mm at approximately 0.975 sec, 6.0 mm at approximately 1.15 sec, 7.0 mm at 1.3 sec, 8.0 mm at approximately 1.567 sec, 9.0 mm at approximately 1.65 sec, and 10.0 mm at 1.8 sec. Although it would take 1.8 sec to record these 11 images, they could be replayed in 1.0 sec, at 10 frames per second.

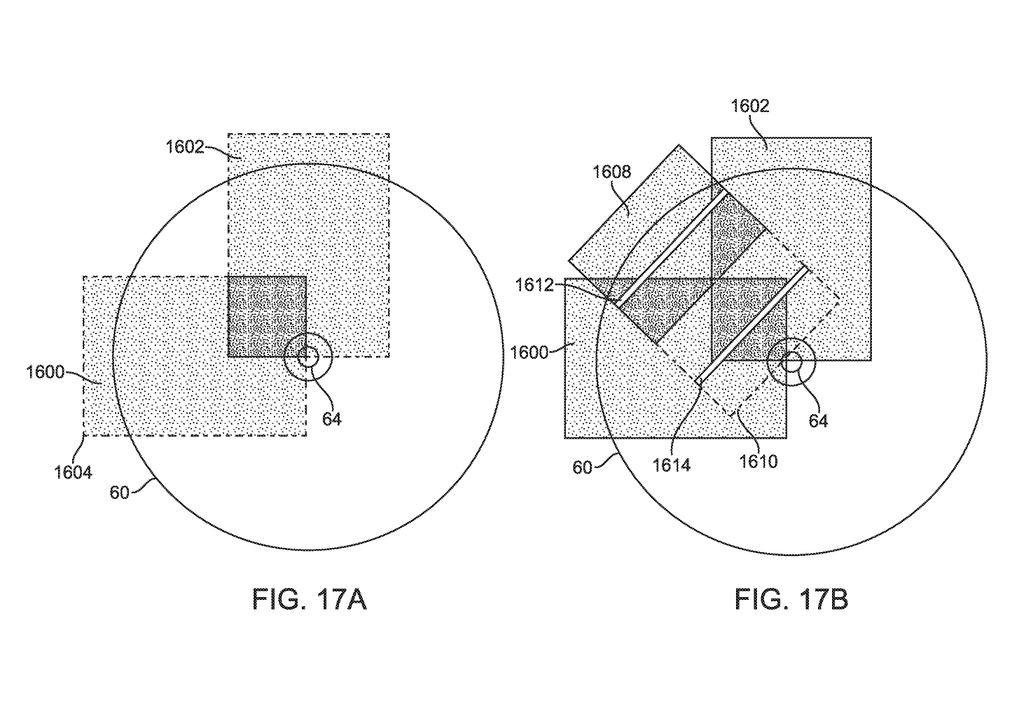

Since the scanning procedure is performed by hand, it is possible that the user, recording the images, may cover the same volume of tissue more than once, recording images for each scan. These overlapping scans can result in redundant images and reviewing those redundant images can increase the review time. In the most elementary description of this phenomenon, if the user scans the same region twice, then the second scan is redundant. Reviewing the second scan would only repeat previously presented information. With the exception of adding a “second” review, it would not serve a clinical purpose to review the second image. In some embodiments, a redundant image is an image for which all of the information contained within that image are contained in other images, or combinations of other images. In the way of example in FIGS. 17A and 17B, the two radial scans 1600 and 1602 of the breast begin at the periphery of the breast 60 and progress to the nipple 64. There is no overlap of scan information on the periphery, but overlap does occur as the scans approach the nipple 64. Any additional images which are recorded within the bounds of the two scans would be redundant. In this example, if a third scan 1608 were obtained between the first two, then, as with the other scans, there would be no overlap of information at the periphery of the breast 60. If a single image 1612 were captured within that portion of the scan, there may be some information that is redundant to other images, but there is other information that has not been imaged. Therefore, this image is not entirely redundant. If the operator continues with that scan, however, he or she will scan a region 1610 which has been completely scanned by the other scans 1600 and 1602. If a single image 1614 were captured in this region then all of the information contained therein would be redundant. In this example the region 1610 may contain a plurality of images, all of which are redundant. Significant review time may be saved by simply not reviewing these images. Some embodiments described provide for reducing review time by determining the overlap or redundancy between images in a scanned set of images. The scan set of images may then be modified to remove overlapping or redundant information. Determining redundancy or overlap may be accomplished by any of the methods described above, for example, by determine distances between pixels or comparing pixel density for scanned images.

In some embodiments, the phrase uniform temporal display or review refers broadly to modifying a scan sequence such that the review time satisfies a predetermined time regardless of the number of images in the scan sequence. In some cases, this is accomplished by allocating dwell times or review times for each image in the scan sequence. For example, a scan sequence having 10 images may have a predetermined review time of 10 seconds for all 10 images. However, the review time allocated to each image within the 10 image scan sequence can vary from image to image. Some images may be assigned 1.0 second dwell times. Other images may be apportioned 0.75 second dwell times. Such allotment may be a function of the relative spacing between the images. In some embodiments, uniform temporal display or review indicates that the overall total time for review of the scan sequence is substantially the same regardless of the individual dwell times or review times for each discrete image within the scan sequence.

In some embodiments, the phrase uniform spatial display or review refers broadly to modifying a scan sequence such that the relative spacing between discrete images within a scan sequence is substantially the same. For example, a scan sequence may have recorded images at 0 mm, 1.0 mm, 1.5 mm, 2.0 mm, 2.2 mm, 2.5 m, and 3.0 mm. Such a scan sequence may be modified to have uniform spatial display or review by removing images that do not have a preferred relative spacing. The relative spacing may be for example 1.0 image-to-image spacing. In this case, the recorded images for review would not include 1.5 mm, 2.2 mm, and 2.5 mm. The modified scan sequence would provide for a uniform spatial display or review.

In some embodiments, the review images may exhibit uniform spatial-temporal display or review having both uniform spatial and uniform temporal characteristics or some combination within the review scan sequence images.

Some embodiments provide for methods, systems, or devices that allow the reviewer to mark or otherwise annotate the images for review. In some cases, the annotation or marking indicates a location on the scanned image that may need to be reviewed further. In other embodiments, the marked section in the image may indicate the site of a suspicious lesion or structure, e.g., potential tumor.

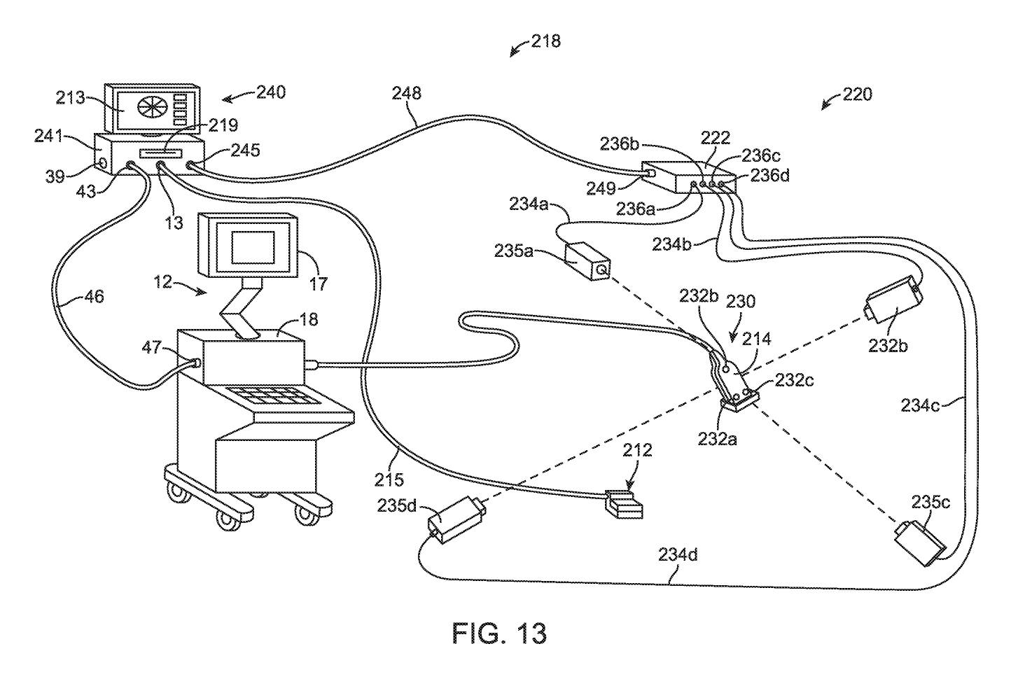

Another embodiment of the present invention is seen in FIG. 13 wherein optical recognition is used for continuously detecting the position and orientation of a hand-held ultrasound probe assembly 230 in place of the use of electromagnetic radiofrequency position sensors as described in the preceding specification related to FIGS. 1 through 9 and FIG. 11. As described previously with regard to FIGS. 1 through 9 and FIG. 11, the optical recognition based position and orientation detection method, apparatus and system is used to accurately determine the position of each two-dimensional ultrasound scan image and, thereby, the temporal position of each pixel within each two-dimensional ultrasound scan image.

Referring to FIG. 13, two principal subsystems are illustrated. A first subsystem is the diagnostic ultrasound system 12, which includes ultrasound monitor console 18, display 17, hand-held ultrasound probe 214 and connecting cable 16. A second system (referred to hereinafter as the “Optically Based Optically Based Ultrasound Scan Completeness Auditing System”), is represented in general at 218. The Optically Based Ultrasound Scan Completeness Auditing System 218 comprises a data acquisition and display module/controller 240 including microcomputer/storage/DVD ROM recording unit 241, display 213 and foot pedal control 212. Foot pedal 212 is connected to microcomputer/storage/DVD ROM recording unit 241 via cable 215 and removably attachable connector 13. The Optically Based Ultrasound Scan Completeness Auditing System 210 also comprises position-tracking system 220, which includes position tracking module 222 and two or more, preferably three or more cameras 235 (e.g., infrared cameras). In addition, the Optically Based Ultrasound Scan Completeness Auditing System 210 also comprises two or more optically unique (i.e., uniquely identifiable) position markers 232 affixed to the hand-held ultrasound probe 214. Said two or more, preferably three or more, cameras may operate in the visible spectrum or infrared spectrum.

By way of example and still referring to FIG. 13, four infrared cameras 235a-235d are shown at predetermined fixed positions whose fields of view include the hand-held ultrasound probe assembly 230 including six optically unique position markers with three position markers 232a-232c visible on the front side of hand-held ultrasound probe assembly 230 (232d-232f on back side of hand-held ultrasound probe assembly 230 but not shown). Said infrared cameras removable connected to position tracking module 222 at connectors 236a-236d via cables 243a-234d. Said optically based position detection method, system and apparatus is capable of obtaining 100 position measurements per second at a camera-to-object distance of up to 3 meters with position accuracies to within less than 1 millimeter. See, for example, an off-the-shelf optically based position detection device, Spotlight Tracker, manufactured by Ascension Technology Corporation, Burlington, Vt.

Still referring to FIG. 13, diagnostic ultrasound system 12 is connected to data acquisition and display module/controller 240 via data transmission cable 46 to enable each frame of ultrasound data (typically containing about 10 million pixels per frame) to be received by the microcomputer/storage/DVD ROM recording unit 241 at the end of each individual scan, which is completed about every 0.1 to 0.02 seconds. Cable 248 is removably attached to microcomputer/storage/DVD ROM recording unit 241 of data acquisition and display module/controller 240 with removably attachable connector 245 and is removably connected to diagnostic ultrasound system 12 with connector 47. The successive scans associated with the diagnostic ultrasound procedure are stored and subjected to computational algorithms to assess completeness of the diagnostic ultrasound scanning procedure as described in greater detail in the specifications which follow.

Still referring to FIG. 13, hand-held ultrasound probe position tracking module 222 is connected to data acquisition and display module/controller 240 via data transmission cable 248 wherein cable 248 is removably attached to microcomputer/storage/DVD ROM recording unit 241 of data acquisition and display module/control 240 with connector 245 and is removably connected to position tracking module with connector 249. Hand-held ultrasound probe assembly 230 seen in FIG. 1 includes, by way of example, six optically unique position markers 232a-232c (232d-232f on back side of hand-held ultrasound probe assembly 230 and not shown), which are affixed to ultrasound hand-held probe 214. As seen in the example arrangement shown in FIG. 13, four infrared cameras 235a-235d are positioned at known locations around the perimeter and in unobstructed view of the hand-held ultrasound probe assembly 230. Optical recognition and vectoring software contained within the position-tracking module 222 provides the exact position and orientation of the hand-held ultrasound probe assembly 230 preferably at time intervals of 0.05 seconds and more preferably of at time intervals of 0.01 seconds.

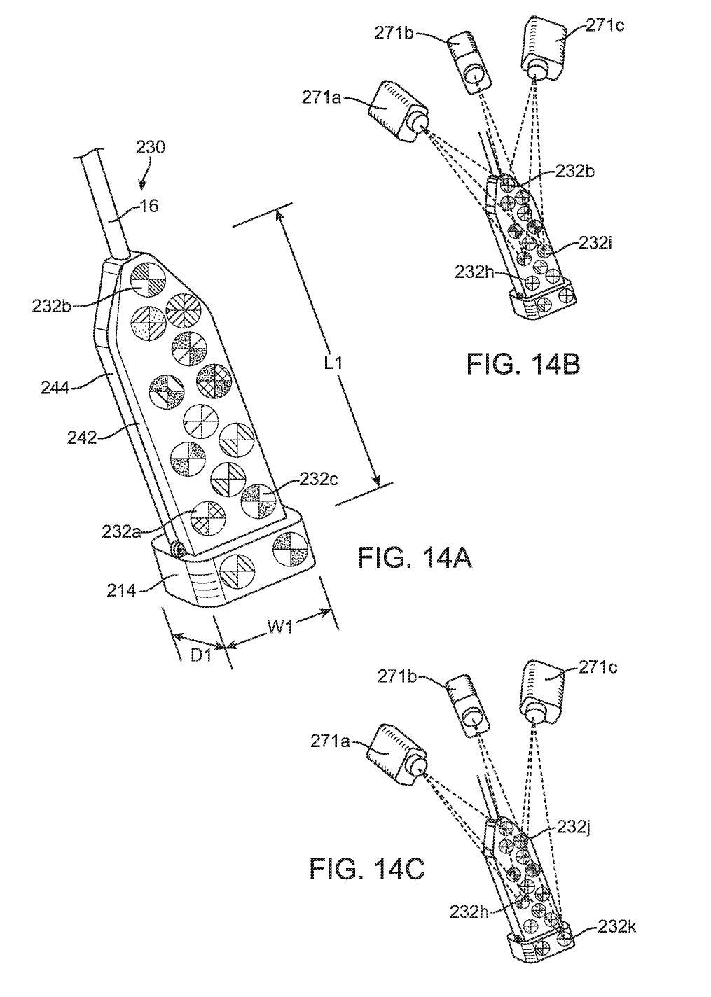

Referring now to FIGS. 14A-14C and by way of example, six optically unique position markers, 232a-232c (232d-232f on back side of hand-held ultrasound probe assembly 230 and not shown) are affixed to the hand-held ultrasound probe 214 as described now in greater detail. These optical position markers can be differentiated from each other by the geometry of the reflective pattern, the reflective wavelength, or a combination therein. In some embodiments, the optical markers can be affixed to the probe assembly 214 by means of an adhesive bond. In another embodiment of a hand-held probe assembly 230, a hand-held ultrasound probe 214 is enclosed within first and second “clamshell” type support members 242 and 244, respectively.

Continuing with this exemplary embodiment and referring to FIGS. 14A-14C, three optically unique position markers 232a-232c are affixed to the exterior surface of first support member 242. In addition, three optically unique position markers 232d-232f (not shown) are affixed to the exterior surface of second support member 244. The number of sensors is only limited by the ability to generate optically unique geometries and colors and the amount of surface area on the probe. Referring to FIG. 14B, three cameras 271a-271c individually locate three markers 232b, 232h, 232i. Since the locations of the markers 232b, 232h, 232i relative to the geometry of the probe assembly 230 are known, the location and calculated orientation of the probe assembly 230 can be determined. The location and calculated orientation of the probe assembly 230 can be determined even if one or more or all of the original markers 232b, 232h, 232i are obscured from the line-of-site of the cameras 271a-271c. As depicted in FIG. 14C, this may be accomplished as the cameras 271a-272c can locate an additional marker such as 232j, 232k for each marker that is obscured 232b, 232i. In some embodiments, the location of three markers 232h, 232j, 232k are known and since the location of these three markers 232h, 232j, 232k are also known relative to the probe assembly 230, the location and the orientation of the probe assembly 230 may be determined. In other embodiments, any number or subset of a plurality of sensors/markers may be used to determine location and orientation of the probe assembly.

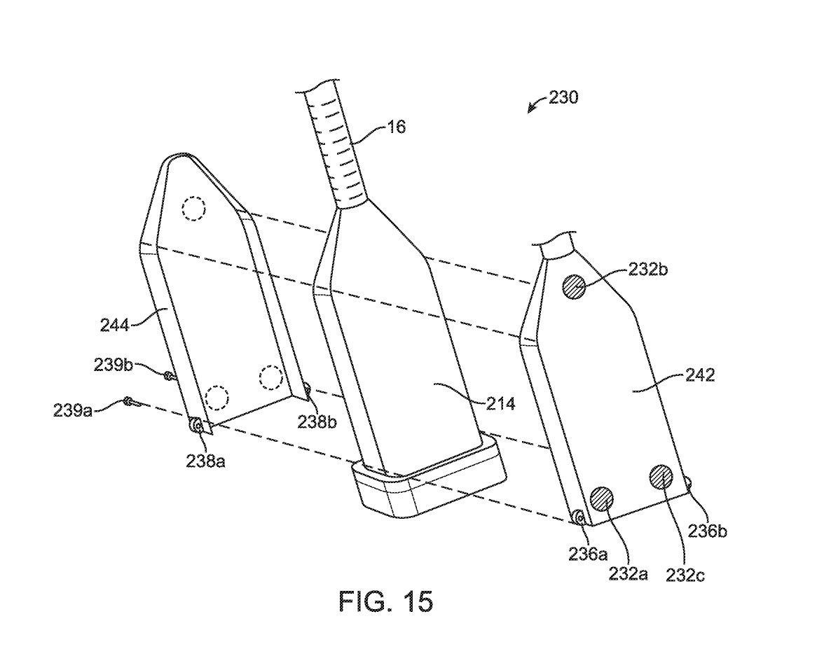

Another embodiment of the present invention is further illustrated in an exploded view of the hand-held probe assembly 230 as seen in FIG. 15. Said first support member 242 includes the aforementioned three optically unique position markers 232a-232c. First support member 242 also incorporates extension ears 236a and 236b, each with a drilled hole to enable secure mechanical attachment to second support member 244. Said second support member 244 likewise incorporates extension ears 238a and 238b, each with a drilled hole which matches drilled holes in first support member to enable secure mechanical attachment to second support member 242 using screws 239a and 239b, respectively. First and second support members may be manufactured using metal, metal alloy or, preferably, a rigid plastic material. The interior contours and dimensions of the first and second support members 242 and 244 are designed to match the particular contour and dimensions of the off-the-shelf hand-held ultrasound probe being instrumented with the optically unique position markers 232a-232c. Accordingly, the contours and dimensions of the first and second support members 242 and 244 will vary according to the hand-held ultrasound probe design. The exact location of the optically unique position markers 232a-232c relative to the ultrasound transducer array at the end face of the hand-held ultrasound probe (not shown) will accordingly be known for each set of first and second support members since they are designed to attached to and operate in conjunction with a specific hand-held ultrasound probe.

Returning to FIG. 2, the typical dimensions of a hand-held ultrasound probe 14 are provided below:
W1=1.5 to 2.5 inches
L1=3 to 5 inches
D1=0.5 to 1 inch

Accordingly, as specified in the previous paragraph, the first and second support members 242 and 244 are sized to correspond to the particular contour and dimensions of a specific hand-held ultrasonic probe design. For the case of injection-molded plastic, e.g., a biocompatible grade of polycarbonate, the inner dimensions of said first and second support members 242 and 244 are designed to closely match the outer dimensions of the hand-held ultrasound probe 214. The wall thickness of the injection molded plastic support members 242 and 244 is preferably in the range from 0.05 to 0.10 inch.

Although certain location and motion recognition methods have been described (e.g. FIG. 13), it can be appreciated that any location and motion recognition methods, software, devices, or systems can be used with the described embodiments. For example, sonar, radar, microwave, or any motion or location detection means may be employed.

Furthermore, a position sensor may not be a separate sensor added to the imaging device but may be a geometric or landmark feature of the imaging device, for example, the corners of the probe. In some embodiments, the optical, infrared, or ultraviolet cameras could capture an image of the probe and interpret the landmark feature as a unique position on the imaging device. Moreover, in some embodiments, sensors may not need to be added to the imaging device. Rather, location and motion detection systems can be used to track the position of the imaging device by using geometric or landmark features of the imaging device. For example, a location system may track the corners or edges of an ultrasound imaging probe while it is scanned across a target tissue.

According to the specifications of embodiments of the present invention, either the electromagnetic radiofrequency-based method, apparatus and system or the optical recognition-based method, apparatus and system can be used to detect the position of the hand-held ultrasound probe at all time points corresponding to the time of any two-dimensional ultrasound scan image. This position and orientation data is used to compute the maximum distance between sequential two dimensional ultrasound scan images to determine if predetermined maximum spacing limits are exceeded or predetermined pixel density limits are not achieved. If any predetermined requirements are not achieved, the ultrasound screening operator is alerted with a visual display identifying that the scan just completed [a] was performed with an excessive spacing relative to the previous scan in the sequence and/or [b] was performed a rate of translation and/or rotation that was too fast to meet pixel density or spacing requirements.

Images may be retrieved and stored in a variety of manners. By way of example and as is one of the teachings in FIG. 1, the microprocessor/storage/DVD ROM recording unit 41 of the data acquisition and display module/controller 40 could be a standard computer with a video frame grabber card. The data transmission cable 46 could connect to the video output of the hand-held imaging system 12 and record discrete images in a wide variety of formats including, but not restricted to JPG, BMP, PNG. Each image would be stored with an information header containing, but not restricted to, the location of the image at the time it was recorded. The individual images could be stored in sets of scan tracks, and the scan tracks could be stored as a complete examination, or the images could be stored using another data management protocol. The resulting set of images could be comprised of several thousand individual, discrete images.

Once the set of images is compiled, it may be stored as a set, along with the location information and other information, such as patient identification, etc., to a portable storage device 9, such as a DVD ROM, portable hard drive, network hard drive, cloud-based memory, etc. These data may be viewed on the data acquisition display module/controller 40, or an external computer equipped with software designed to review the image data.

In yet another embodiment of the present invention, an optical image projector can be included in either the Ultrasound Scan Completeness Auditing System or the Optically Based Ultrasound Scan Completeness Auditing System to superimpose optical information on the surface of the targeted tissue (e.g., the human female breast). Said optical information may, by way of example, include the ultrasound scan path(s) that need to be repeated due to excessive inter-scan distances, inadequate overlap and/or excessive scanning translation speed and/or rate of rotation. Said optical information can thereby guide the conduct of additional two-dimensional ultrasound scans to overcome any determined deficiencies.

Since certain changes may be made in the above-described system, apparatus and method without departing from the scope of the invention herein involved, it is intended that all matter contained in the description thereof or shown in the accompanying drawings shall be interpreted as illustrative and not in a limiting sense. The disclosed invention advances the state of the art and its many advantages include those described herein.

As for additional details pertinent to the present invention, materials and manufacturing techniques may be employed as within the level of those with skill in the relevant art. The same may hold true with respect to method-based aspects of the invention in terms of additional acts commonly or logically employed. Also, it is contemplated that any optional feature of the inventive variations described may be set forth and claimed independently, or in combination with any one or more of the features described herein. Likewise, reference to a singular item, includes the possibility that there are plural of the same items present. More specifically, as used herein and in the appended claims, the singular forms “a,” “and,” “said,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs. The breadth of the present invention is not to be limited by the subject specification, but rather only by the plain meaning of the claim terms employed.

1. (canceled)

2. A scan completeness auditing system for use with an ultrasound imaging console in screening a volume of tissue comprising:
a position tracking system configured to track and record the position of a manual ultrasonic imaging probe, the position tracking system comprising a plurality of position sensors adapted to couple to a manual ultrasonic imaging probe, the position sensors configured to provide position data for the manual ultrasonic imaging probe; and
a receiver comprising a controller configured to
electronically receive position data for the manual ultrasonic imaging probe from the position tracking system,
electronically receive and record a first scan sequence comprising a first set of scanned images representing cross-sections of the tissue from the manual imaging probe,
assign a replay dwell time to each image in the first scan sequence, the dwell time for each image being based on a relative spacing for that image in the first scan sequence computed from the position data,
compute image-to-image spacing between successive images within the first scan sequence, and
provide an alert when the computed spacing exceeds a maximum limit.

3. The system of claim 2, wherein the controller applies an image position tracking algorithm to determine a relative resolution between the scanned images within the scan sequence.

4. The system of claim 2, wherein the controller is configured to measure a scan-to-scan spacing between the first scan sequence and a second scan sequence, the second scan sequence comprising a second set of scanned images representing cross-sections of the tissue.

5. The system of claim 4, wherein the controller is configured to measure a scan-to-scan spacing between the first and second scan sequence by calculating the distance between a first boundary of the first scan sequence and a second boundary of the second scan sequence.

6. The system of claim 4, wherein the controller is configured to measure a scan-to-scan spacing between the first and second scan sequences by computing a pixel density for a unit volume within the screened volume of tissue and comparing the computed pixel density to a minimum pixel density value, the controller configured to provide an alert to rescan the tissue if the computer pixel density is less than the minimum pixel density value.

7. The system of claim 4, wherein the controller is configured to modify the first or second scan sequences for display by removing redundancy from at least one of the scan sequences.

8. The system of claim 2, wherein the controller is configured to compute the image-to-image spacing between scanned images within a scan sequence by measuring a distance between a first pixel in a first scanned image and a second pixel in a second scanned image, wherein the first and second scanned images are sequential images.

9. The system of claim 8, wherein the controller is configured to determine whether the measured distance between the first and second pixels exceeds a maximum distance.

10. The system of claim 2, wherein the controller is configured to compute the image-to-image spacing within the first scan sequence by measuring a maximum chord distance between a plurality of successive planar images in the first scan sequence.

11. The system of claim 2, wherein the controller is configured to compute the image-to-image spacing within the first scan sequence by calculating a pixel density for a unit volume within the screened volume of tissue, and the controller adapted to compare the calculated pixel density with a minimum pixel density value.

12. The system of claim 11, wherein the minimum pixel density value is between 9,000 pixels/cm³ to 180,000,000 pixels/cm³.

13. The system of claim 2, wherein the controller is configured to only display images of a recorded scan sequence that satisfy a predetermined imaging spacing interval.

14. The system of claim 2, wherein the controller is configured to change an image display rate of a recorded scan sequence to provide a substantially uniform spatial-temporal display of the recorded scan sequence.

15. The apparatus of claim 2, wherein the receiver includes a cable configured to engage with a video output of the ultrasound imaging console.

16. The system of claim 2 wherein the controller is further configured to derive orientation data for the manual ultrasonic imaging probe from the position data.

17. A method for screening tissue, comprising:
scanning the tissue with a manual ultrasonic imaging probe of an ultrasound imaging console along a first scanning path on the tissue;
generating a first scan sequence comprising a first set of discrete digital images representing cross-sections of the scanned tissue along the first scanning path;
electronically transmitting the first scan sequence to a controller;
electronically communicating position data for the manual ultrasonic imaging probe to the controller, wherein the position data is collected from a plurality of position sensors;
assigning a display dwell time to each image based on a relative spacing for that image in the first scan sequence computed from the position data;
computing image-to-image spacing between successive images within the first scan sequence, and
generating an alert when the computed spacing exceeds a maximum limit.

18. The method of claim 17 further comprising:
computing an image-to-image spacing between successive images in the first scan sequence based on the position data communicated to the controller;
determining whether the image-to-image spacing exceeds a maximum limit; and
generating an alert when the spacing exceeds a maximum limit.

19. The method of claim 18, further comprising:
generating a second scan sequence, the second scan sequence comprising a second set of discrete digital images along a second scanning path on the tissue;
computing a scan-to-scan spacing between the first and second scan sequences;
determining whether the computed scan-to-scan spacing exceeds a scan-to-scan spacing limit; and
generating an alert when the scan-to-scan spacing exceeds the scan-to-scan spacing limit.

20. The method of claim 19, wherein the image-to-image spacing and the scan-to-scan spacing are calculated based on the position data communicated to the controller and orientation data derived from the communicated position data.

21. The method of claim 19, wherein each of the images in the first and second sets of discrete digital images comprises a matrix of pixels, each matrix having the same fixed number of rows and columns and each pixel in each matrix having a row and column location designed by rₓ, cₓ, x being the same or different for r and c, wherein computing the scan-to-scan spacing between the first and second scan sequences comprises calculating a plurality of pixel-to-pixel distances between a first pixel P(rₓ, cₓ) in a first image of the first scan sequence and a plurality of pixels in the second scan sequence, wherein the plurality of pixels in the second scan sequence have the same row location rₓ as the first pixel P.

22. The method claim 19 further comprising removing a redundant image from the first scan sequence or the second scan sequence.

23. The method of claim 18, wherein the computing an image-to-image spacing step comprises calculating a pixel density for a unit volume of the screened tissue; and the determining step comprises comparing the calculated pixel density to a minimum pixel density value.

24. The method of claim 18, wherein computing the image-to-image spacing step comprises calculating a maximum chord distance between images in the first scan sequence.

25. The method of claim 18, wherein computing the image-to-image spacing within the first scan sequence comprises:
calculating a maximum pixel distance between a first image and a second image of the first scan sequence, the first image having a first pixel matrix and the second image having a second pixel matrix, wherein the first and second pixel matrices each have the same number of rows and columns; and
determining the maximum pixel distance by measuring a pixel-to-pixel distance between at least two corresponding pixels, wherein one of the at least two corresponding pixels is in the first pixel matrix and the other of the at least two corresponding pixels is in the second pixel matrix, the corresponding pixels having the same row and column locations in respective matrices.

26. The method of claim 25, wherein determining the maximum pixel distance comprises computing the pixel-to-pixel distance between a corner pixel on the first pixel matrix and a corresponding corner pixel on the second pixel matrix.

27. The method of claim 17 further comprising deriving orientation data for the manual ultrasonic imaging probe based on the position data communicated to the controller.

28. The method of claim 17, further comprising prior to scanning, attaching the plurality of position sensors to the manual ultrasonic probe.

29. The method of claim 17, wherein the first scan sequence is transmitted from a video output of an ultrasound imaging console in communication with the ultrasonic imaging probe to the controller.

30. The method of claim 29, further comprising prior to scanning, attaching a cable to the video output of the ultrasound imaging console to the controller, wherein the first scan sequence is electronically transmitted by the cable.
